SEQUENCE LISTING (126 sequences)

SEQ ID NO: 1 - DNA, 2794 bp, homo sapiens
agagaaccatcattaattgaagtgagatttttctggcctgagacttgcagggaggcaagaagacactctggacaccactatggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttgatagcaacttccaggaatgtcacacacgatgaaatatctctgctgaagacagtggataaaaaacagtccttcaagtcttctctgtttttattcttcaactctcactttcttagagtttacagaaaaaatatttatatacgactctttaaaaagatctatgtcttgaaaatagagaaggaacacaggtctggccagggacgtgctgcaattggtgcagttttgaatgcaacattgtcccctactgggaataacagaactgcaggacctgggagcatcctaaagtgtcaacgtttttctatgacttttaggtaggatgagagcagaaggtagatcctaaaaagcatggtgagaggatcaaatgtttttatatcaacatcctttattatttgattcatttgagttaacagtggtgttagtgatagatttttctattcttttcccttgacgtttactttcaagtaacacaaactcttccatcaggccatgatctataggacctcctaatgagagtatctgggtgattgtgaccccaaaccatctctccaaagcattaatatccaatcatgcgctgtatgttttaatcagcagaagcatgtttttatgtttgtacaaaagaagattgttatgggtggggatggaggtatagaccatgcatggtcaccttcaagctactttaataaaggatcttaaaatgggcaggaggactgtgaacaagacaccctaataatgggttgatgtctgaagtagcaaatcttctggaaacgcaaactcttttaaggaagtccctaatttagaaacacccacaaacttcacatatcataattagcaaacaattggaaggaagttgcttgaatgttggggagaggaaaatctattggctctcgtgggtctcttcatctcagaaatgccaatcaggtcaaggtttgctacattttgtatgtgtgtgatgcttctcccaaaggtatattaactatataagagagttgtgacaaaacagaatgataaagctgcgaaccgtggcacacgctcatagttctagctgcttgggaggttgaggagggaggatggcttgaacacaggtgttcaaggccagcctgggcaacataacaagatcctgtctctcaaaaaaaaaaaaaaaaaaaagaaagagagagggccgggcgtggtggctcacgcctgtaatcccagcactttgggaggccgagccgggcggatcacctgtggtcaggagtttgagaccagcctggccaacatggcaaaaccccgtctgtactcaaaatgcaaaaattagccaggcgtggtagcaggcacctgtaatcccagctacttgggaggctgaggcaggagaatcgcttgaacccaggaggtggaggttgcagtaagctgagatcgtgccgttgcactccagcctgggcgacaagagcaagactctgtctcagaaaaaaaaaaaaaaaagagagagagagagaaagagaacaatatttgggagagaaggatggggaagcattgcaaggaaattgtgctttatccaacaaaatgtaaggagccaataagggatccctatttgtctcttttggtgtctatttgtccctaacaactgtctttgacagtgagaaaaatattcagaataaccatatccctgtgccgttattacctagcaacccttgcaatgaagatgagcagatccacaggaaaacttgaatgcacaactgtcttattttaatcttattgtacataagtttgtaaaagagttaaaaattgttacttcatgtattcatttatattttatattattttgcgtctaatgattttttattaacatgatttccttttctgatatattgaaatggagtctcaaagcttcataaatttataactttagaaatgattctaataacaacgtatgtaattgtaacattgcagtaatggtgctacgaagccatttctcttgatttttagtaaacttttatgacagcaaatttgcttctggctcactttcaatcagttaaataaatgataaataattttggaagctgtgaagataaaataccaaataaaataatataaaagtgatttatatgaagttaaaataaaaaatcagtatgatggaataaacttg

SEQ ID NO: 2 - protein, 198 aa, homo sapiens [sequence omitted]

SEQ ID NO: 3 - DNA, 911 bp, homo sapiens
gactccagaggaggaagtccagagacagagcaccatgacttctgagaaaggtccttcaaccggtgaccccactctgaggagaagaatcgaaccctgggagtttgacgtcttctatgaccccagagaacttcgtaaagaggcctgtctgctctacgaaatcaagtggggcatgagccggaagatctggcgaagctcaggcaaaaacaccaccaatcacgtggaagttaattttataaaaaaatttacgtcagaaagagattttcacccatccatgagctgctccatcacctggttcttgtcctggagtccctgctgggaatgctcccaggctattagagagtttctgagtcggcaccctggtgtgactctagtgatctacgtagctcggcttttttggcacatggatcaacaaaatcggcaaggtctcagggaccttgttaacagtggagtaactattcagattatgagagcatcagagtattatcactgctggaggaattttgtcaactacccacctggggatgaagctcactggccacaatacccacctctgtggatgatgttgtacgcactggagctgcactgcataattctaagtcttccaccctgtttaaagatttcaagaagatggcaaaatcatcttacatttttcagacttcatcttcaaaactgccattaccaaacgattccgccacacatccttttagctacagggctgatacatccttctgtggcttggagatgaataggatgattccgtgtgtgtactgattcaagaacaagcaatgatgacccactaaagagtgaatgccatttagaatctagaaatgttcacaaggtaccccaaaactctgtagcttaaaccaacaataaatatgtattacctctggcaaaaaaaaaaaaaaaaaaaa

SEQ ID NO: 4 - protein, 236 aa, homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 5 - DNA, 4706 bp, homo sapiens
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgtattcccagcctgagcaccacgcagaaatgtgcttcctctcttggttctgtggcaaccagctgcctgcttacaagtgtttccagatcacctggtttgtatcctggaccccctgcccggactgtgtggcgaagctggccgaattcctggctgagcaccccaatgtcaccctgaccatctccgccgcccgcctctactactactgggaaagagattaccgaagggcgctctgcaggctgagtcaggcaggggcccgcgtgaagattatggacgatgaagaatttgcatactgctgggaaaactttgtgtacagtgaaggtcagccattcatgccttggtacaaattcgatgacaattatgcattcctgcaccgcacgctaaaggagattctcagaaacccgatggaggcaatgtatccacacatattctacttccactttaaaaacctacgcaaagcctatggtcggaacgaaagctggctgtgcttcaccatggaagttgtaaagcaccactcacctgtctcctggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctactacttctgggatacagattaccaggaggggctccgcagcctgagtcaggaaggggcctccgtggagatcatgggctacaaagattttaaatattgttgggaaaactttgtgtacaatgatgatgagccattcaagccttggaaaggactaaaatacaactttctattcctggacagcaagctgcaggagattctcgagtgaggggtctccccgggcctcatggtctgtctcctctagcctcctgctcatgttgtgcaggcctcccctccatcctggaccagctgtgcttttgcctggtcatcctgagcccctcctggcctcagggccattccatagtgctcccctgcctcaccacctcctctccgctctcccaggctcttcctgcagaggcctctttctgcctccatggctatccatccacccaccaagaccctgttccctgagcctgcatgcccctaacctgccttttcccatctccccagcataacctaatattttttttttttttttgagacggaatttcgctctgtcacccagactggagtgcaatggcttgatcttggctcactgcaaactctgcctaccaggttcaagcgattctcctgcctccgcctcccgagtagctggaattacagacgcctgccaccacgcacagctaactttttttttttttgtatttttagtagtgactgggtttcaccatgttggccaggctggtcttgaactcctgacctcaggtgatccgcctatctcagcctcccaaagtgctgggattacaggcgtgagccactggcccggcggcacaaccaaatcttattaaactcaccctaggctggccgcggtgactcatgcctataatcccccagcaatttgggaggcagaggtgagagaatcgcttgagcccaggaattcgagaccagcctgggccacatgacaaagccccatctctacaaaaaaattacaaaaaaaaaaaaaacaggtgtggtggcatgcacctgtagttgaagctacttggaaggatgaagtgggaggattgcttgagccggggaggtggaggctgcagtgaactgagatcacgtcactgaactccagtctgagcaacagatcgagaccctgcctgaaaataaatcaataaataaactcaaccgaaatgggtatgaaagttgaaatgggtatgtaagttgaaaaccagaagttttgagaaacatcctttgttaactttcatcctacaaattgggtcattcatgtcctacgcagctaaaacagagcccaggagccagggaggaaaagcagtcaggccacacaccattgctcccaaaatggacttctctgcaagcctgactcctgaaactgtgcattgtaccctgaaaccagctttatccatagcttctgcaataaatggctgtaagtcttggactccttgctataatcgcagctattcagcaatggaacctcccagttcccaacccttcctagtgcccatgggctttcccataggacaagagaacatttctccttttctttttttttttctttgaaatggagtctcgccctgtcacccaggctggagtgcaatggtgcggtctcggctcactgcaacctctgcctcccttgttcaagtgattctcctgtctcagcctcccgagtagctgggattacaggcgtccaccaccaaaccaggctaatttttgtatttttcataaaaacgggtttcatcatgtttcccaggctggtcttatttttattttattttttgagatggagtcttgctctgttgcccaggctggggtgcagtggtgcaatctgggttcactgcagcctctgccgcctgagttcaagctattttcctacctaagcctcccaagtagctgggattacatgcgcgtgccaccacgcctagctaatttttgtgtttttagtagagacggggtttcaacatcttgaccaggctggtcttgaactcctgacctcgtgatccacccgtctcggcctcccaaagtgctgggattacaggcgtgagccacctggccaggcttaggctggtcttaaactcctgacctcaagtgatccaacctccttggcctcccaaattgctgggattgctggtgtgagccacagcgcctagcccatttctccttttaataggacctgttgctgtctctgttctcccaacatggtgaacaccacccggactgcgtgtatgtcccaaattacaattctttctttgcaaatgaaatgtgaaatttagaggcccttctccacactttaaatttgacttgacattttctaggcagatataagttattagagaatgagattctctataaaaatgatcccttcatgctgtggcctccacagaagatgccctgggccaggtgcccacatgaataatgcgggccacaggcaggcatttattttctcacagatatggaggctacaagtccaaggtggaggggtcggcggggttgtttgctctgaggccgctcctcctggatggcagggatcccttctggctgtgtcctctgtggcctttcctctatgaacctgtactgtacctctggggtctctctgcttccaaatatcttttttttttttttcagacagttttgctcttgttttctaggctggagtgcaatggcacaatctcagctcactgcaacctctgccttccgagttcaagcgattctcgtgcctcagcctcctgagtagctgggactacaggcgtgtgccaccacgcctggctaattttgtagttttagtagagacggggtttctccatgttgctcaggctggtcttgaactcatgagctcaggcgatccactctcctcagcctcccaaagtgctgggattacagatataagccaccatacacaacttttttttttttttgagatggagtttcactctgttgcccaggctggagtgctaaatagcagaatcactgctcactgcaacctctgcctgctgggttcaagcaattctcccacctcagcctcctgagtagctgggattacagatgcccagaaccaatctctgctaatttttctattttttagtagagatggggtttcactgaggaaggagaccacctctctcattgtctcctatttcagaaggaagcaaaaagttagaaagatgcagaagtaagatcaatggccagactgtttggcgctgctacctgggcctggtagttaaagatcaactcctgacctgaccgcttgttttatctaaagattccagacattgtatgaggaagcattgtgaaactttctggtctgttctgctagcccccaccactgatgcatgtagccccccagtcacgtagcccacgcttgcacaatctatcacgaccctttcacgtggaccccttagaattgtaagcccttaaaagggccagggacttcttcagggagctccaatcttcagatgcaagtctgtcaacgctcccagctgattaaagcctcttccttcctaaaaaaaaaaaaaaaaaa

SEQ ID NO: 6 - protein, 373 aa, homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 7 - DNA, 822 bp, homo sapiens
ttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagggggaggggccatgactacgaggccctgggaggtcactttagggagggctgtcctgaaacctggagcctggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccgtttggacgcaaagatctttcgaggccaggtgcccaggtctttcatcagagccccatttcaagtgctcagtagcccctttggccagtgcgccccaccacatgggacagcgcaggtccagtggcctccccagctgaccgcaggcagggaacaaggcagaccctagagggccaggccacagcaggggctgaggatgcctggtgaatggatgcctgggagaatggatgccagaattcacgcatgaggctctgaacagggctgggaaaacttccaaacgaagggaagctcatgtcttggtgcactttgtgatgatgcttcaacagcaggactgagatggggacatttacaataaacagaaatgtatgggctcgaaaaaaaaaaaaaaaaaa

SEQ ID NO: 8 - protein, 101 aa, homo sapiens
MKPHFRNTVERMYRDTFSYNFYNRPILSRRNTVWLCYEVKTKGPSRPRLDAKIFRGQVPRSFIRAPFQVLSSPFGQCAPPHGTAQVQWPPQLTAGREQGRP

SEQ ID NO: 9 - DNA, 1828 bp, homo sapiens
agcctggtgtggacccacctcccgggcgctggctgcaatgactttctctttccctttgcaattgccttgggtcctgccgcacagagcggcctgtctttatcagaggtccctctgccagggggagggccccagagaaaaccagaaagagggtgagagactgaggaagataaagcgtcccagggcctcctacaccagcgcctgagcaggaagcgggaggggccatgactacgaggccctgggaggtcactttagggagggctgtcctaaaaccagaagcttggagcagaaagtgaaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatctttgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaatacacagaaaagtttcaaacctactaatccagcgacaatttgaatcggttttgtaggtagaggaataaaatgaaatactaaatctttctgtaaaaaaaaaa

SEQ ID NO: 10 - protein, 384 aa, homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 11 - DNA, 1164 bp, homo sapiens
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccaaagtttgctgactgctgggaaaactttgtggaccacgagaaaccgctttccttcaacccctataagatgttagaggagctagataaaaacagtcgagccataaagcgacggcttgagaggataaagattccaggggtacgtgcgcagggtcgttacatggatatattgtgtgatgctgaggtctgagtcacccaatctactggaacatagcacccaatagcagtcctgaagtgtggatgttttagagaatgacttaagaagtttgcagcttggacccgtatcccactcattatcaagaagcaactcaagatgactttccctggggcatgtcagttgcctcatagcctgctggtcctgtaagcaagcactaagctccacagtgccagttccttgccccaacctggccccatccaagtacagaagaccttcctttcctcctttttccatattgctttctgttctaagtgggtgaataattttataattgaaaaaataaagataaagtctgtaaatccagccgggtacggtggctcacgcctgtaatcctagcactttgggaggctgagatgctcggccaataaatttctattgtttatgaaaaaaaaaaaaaaaaaaaaaaa

SEQ ID NO: 12 - protein, 200 aa, homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 13 - DNA, 1070 bp, homo sapiens
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccaaagtttgctgactgctgggaaaactttgtggaccacgagaaaccgctttccttcaacccctataagatgttagaggagctagataaaaacagtcgagccataaagcgacggcttgagaggataaagcagtcctgaagtgtggatgttttagagaatgacttaagaagtttgcagcttggacccgtatcccactcattatcaagaagcaactcaagatgactttccctggggcatgtcagttgcctcatagcctgctggtcctgtaagcaagcactaagctccacagtgccagttccttgccccaacctggccccatccaagtacagaagaccttcctttcctcctttttccatattgctttctgttctaagtgggtgaataattttataattgaaaaaataaagataaagtctgtaaatccagccgggtacggtggctcacgcctgtaatcctagcactttgggaggctgagatgctcggccaataaatttctattgtttatgaaaaaaaaaaaaaaaaaaaaaaa

SEQ ID NO: 14 - protein, 183 aa, homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKQS

SEQ ID NO: 15 - DNA, 1067 bp, homo sapiens
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccaaagtttgctgactgctgggaaaactttgtggaccacgagaaaccgctttccttcaacccctataagatgttagaggagctagataaaaacagtcgagccataaagcgacggcttgagaggataaagtcctgaagtgtggatgttttagagaatgacttaagaagtttgcagcttggacccgtatcccactcattatcaagaagcaactcaagatgactttccctggggcatgtcagttgcctcatagcctgctggtcctgtaagcaagcactaagctccacagtgccagttccttgccccaacctggccccatccaagtacagaagaccttcctttcctcctttttccatattgctttctgttctaagtgggtgaataattttataattgaaaaaataaagataaagtctgtaaatccagccgggtacggtggctcacgcctgtaatcctagcactttgggaggctgagatgctcggccaataaatttctattgtttatgaaaaaaaaaaaaaaaaaaaaaaa

SEQ ID NO: 16 - skipped

SEQ ID NO: 17 - DNA, 639 bp, homo sapiens
tgacttttgggagagctgaccttttgtgacttttgggagagctgccaaaagtgaaacttagtgcctcagacaagcaggggcaagtctgctaaggaagctgtggccagaagcacagatcagaaacacgatggctctgttaacagccgaaacattccgcttacagtttaacaacaagcgccgcctcagaaggccttactacccgaggaaggccctcttgtgttaccagctgacgccgcagaatggctccacgcccacgagaggctactttgaaaacaagaaaaagtgccatgcagaaatttgctttattaacgagatcaagtccatgggactggacgaaacgcagtgctaccaagtcacctgttacctcacgtggagcccctgctcctcctgtgcctgggagctggttgacttcatcaaggctcacgaccatctgaacctgggcatcttcgcctcccgcctgtactaccactggtgcaagccccagcagaaggggctgcggcttctgtgtggatcccaggtcccggtggaggtcatgggcttcccaaattccaggggtacgtgcgcagggtcgttacatggatatattgtgtgatgctgaggtctgagtcacccaatctactggaacatagcacccaatag

SEQ ID NO: 18 - protein, 154 aa, homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPNSRGTCAGSLHGYIV

SEQ ID NO: 19 - DNA, 1643 bp, homo sapiens
acgaagcctggcctgctgggtccttttcccgtcatccccagccagatttagctgctgacagctgcttgggactctgccgccagggcctggcccagacctgcctgcctctctcctctccctcagtgactcctgagccacagcccctccatggcccagaaggaagaggctgctgtggccactgaggctgcctcccagaatggggaggatctggagaacctggacgaccctgagaagctgaaagagctgattgagctgccgccctttgagattgtcacaggagaacggctgcctgccaacttctttaaattccagttccggaatgtggagtacagttccgggaggaacaagaccttcctctgctatgtggttgaagcacagggcaaggggggccaagtgcaggcatctcggggatacctagaggatgagcatgcggctgcccatgcagaggaagctttcttcaacaccatcctgccagccttcgacccagccctgcggtacaatgtcacctggtatgtgtcctccagcccctgtgcagcgtgtgctgaccgcattatcaaaacccttagcaagaccaagaacctgcgtctgctcattctggtgggtcgactcttcatgtgggaggagccggagatccaggctgctctgaagaagctgaaggaggctggctgtaaactgcgcatcatgaagccccaggacttcgaatatgtctggcagaattttgtggagcaagaagagggtgaatccaaggcctttcagccctgggaggacattcaggagaacttcctatactacgaggagaagttggcagacatcctgaagtagggcaactgggctttgcctcacgtattcctgctgccaccaagagacagcaatgacatgtacagccatctgggacatgcctgtcttcctaataccatttggagctggacaacatttgacaccaaccaatcatactggacaaggcccttagaggacttgaaatatacttctcatgctgtagtttatttaggctgtgactctctctctaatgctgctctcgggaaggacgaaagtgacctgcaaggagagaaatgcaaccatacatgggctccagtcaactatgggactgaaggtcctaattgctcacccaagggggctgcttaacacaaacagcctcagacccgaggtttagatttctgaaatatgcattttatgttaagttgggtatttttttaaaaaaagaaaaacagcaacattaataaaagaagtggtgtgtttttcccgtggccagatttttaagaaacttgaacatctggaagttgccacagacaagcgagctagtccagacacagggacactccaggctatgaaccaagccatggttaaaccaagacttgcctagacaatgcagaataactctcccatacagacttcttacgacaatcaagtgagggcctttcccttctctgaaggcaagtaatagggcagaaggtggaacaaaaagcatgagaaatctaaggtagccacatcccaaatgccagttaataagcaactagcttctttattagagaggagcgggtgaaaagtagtagtttaatgttcagatatacaatcatttaaaagtattcaatacatggtgtttgatggtaatggcaaaaaaaaaa

SEQ ID NO: 20 - protein, 224 aa, homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 21 - DNA, 1444 bp, homo sapiens
ggagaaggggtggggcagggtatcgctgactcagcagcttccaggttgctctgatgatatattaaggctcctgaatcctaagagaatgttggtgaagatcttaacaccacgccttgagcaagtcgcaagagcgggaggacacagaccaggaaccgagaagggacaagcacatggaagccagcccagcatccgggcccagacacttgatggatccacacatattcacttccaactttaacaatggcattggaaggcataagacctacctgtgctacgaagtggagcgcctggacaatggcacctcggtcaagatggaccagcacaggggctttctacacaaccaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgtatcttcgctgcccgcatctatgattacgaccccctatataaggaggcactgcaaatgctgcgggatgctggggcccaagtctccatcatgacctacgatgaatttaagcactgctgggacacctttgtggaccaccagggatgtcccttccagccctgggatggactagatgagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacgccagcaaagcagtatgctcccgatcaagtagatttttaaaaaatcagagtgggccgggcgcggtggctcacgcctgtaatcccagcactttggaggccaaggcgggtggatcacgaggtcaggagatcgagaccatcctggctaacacggtgaaaccctgtctctactaaaaatacaaaaaattagccaggcgtggtggcgggcgcctgtagtcccagctactctggaggctgaggcaggagagtagcgtgaacccgggaggcagagcttgcggtgagccgagattgcgctactgcactccagcctgggcgacagtaccagactccatctcaaaaaaaaaaaaaccagactgaattaattttaactgaaaatttctcttatgttccaagtacacaatagtaagattatgctcaatattctcagaataattttcaatgtattaatgaaatgaaatgataatttggcttcatatctagactaacacaaaattaagaatcttccataattgcttttgctcagtaactgtgtcatgaattgcaagagtttccacaaacact

SEQ ID NO: 22 - protein, 199 aa, homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 23 - DNA, 1536 bp, homo sapiens
acagagcttcaaaaaaagagcgggacagggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggccaggtgtatttcaagcctcagtaccacgcagaaatgtgcttcctctcttggttctgtggcaaccagctgcctgcttacaagtgtttccagatcacctggtttgtatcctggaccccctgcccggactgtgtggcgaagctggccgaattcctgtctgagcaccccaatgtcaccctgaccatctctgccgcccgcctctactactactgggaaagagattaccgaagggcgctctgcaggctgagtcaggcaggagcccgcgtgacgatcatggactatgaagaatttgcatactgctgggaaaactttgtgtacaatgaaggtcagcaattcatgccttggtacaaattcgatgaaaattatgcattcctgcaccgcacgctaaaggagattctcagatacctgatggatccagacacattcactttcaactttaataatgaccctttggtccttcgacggcgccagacctacttgtgctatgaggtggagcgcctggacaatggcacctgggtcctgatggaccagcacatgggctttctatgcaacgaggctaagaatcttctctgtggcttttacggccgccatgcggagctgcgcttcttggacctggttccttctttgcagttggacccggcccagatctacagggtcacttggttcatctcctggagcccctgcttctcctggggctgtgccggggaagtgcgtgcgttccttcaggagaacacacacgtgagactgcgcatcttcgctgcccgcatctatgattacgaccccctatataaggaggcgctgcaaatgctgcgggatgctggggcccaagtctccatcatgacctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacagaccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaataaaacattaagaatcttccataattgtttccacaaacact

SEQ ID NO: 24 - protein, 382 aa, homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 25 - DNA, 1127 bp, homo sapiens
ttaaagagggctgctcaactgcaaggacgctgtaagcaggaagagaagccacagcgcttcagaaaagagtgggacagggacaagcatatctaagaggctgaacatgaatccacagatcagaaacccgatgaaggcaatgtatccaggcacattctacttccaatttaaaaacctatgggaagccaacgatcggaacgaaacttggctgtgcttcaccgtggaaggtataaagcgccgctcagttgtctcctggaagacgggcgtcttccgaaaccaggtggattctgagacccattgtcatgcagaaaggtgcttcctctcttggttctgcgacgacatactgtctcctaacacaaagtaccaggtcacctggtacacatcttggagcccttgcccagactgtgcaggggaggtggccgagttcctggccaggcacagcaacgtgaatctcaccatcttcaccgcccgcctctactacttccagtatccatgttaccaggaggggctccgcagcctgagtcaggaaggggtcgctgtggagatcatggactatgaagattttaaatattgttgggaaaactttgtgtacaatgataatgagccattcaagccttggaagggattaaaaaccaactttcgacttctgaaaagaaggctacgggagagtctccagtgaggggtctccctgggcctcatggtctgtctcctctagcctcctgctcatgctgcacgggcctcccctccaccctggacccgctctgtttctgcctggtcatcctgagcccctcctggcctcagggccattccacagtgctcccctgcctcaccgcttcctcctcgctcttccagactcttcctgcagaggctcctttctgcctccatggctatccatccacccccacagaccccgttcctccagcctgcgtgcccctaacctggcttttcccatctccccagcataaccaaatcttactaaactcatcctaggctgggcatggtgactcacgcctgtaatcccccagcaatttgggaggcaaaggtgggagaatcgcgtgagcccaggagttccagaccaggctgggtcacatgacaaagccccatctctacaaaaaaaaaaaaaaaaaaaa

SEQ ID NO: 26 - protein, 190 aa, homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 27 - DNA, 2519 bp, homo sapiens
tttttttttttttgagaggaagcctcgctctctcacccaggctggagtgcagtggcaggaacttggctcactgcaacctccgcttcccaggttcaagtgattctcctgcctcagcctccagagtagctgggattacaggcgcacgtcaccacacctggctaatttttgtatttttagtagagacggggtttctccatgttggtcaggctggtctcgaactcctgacctcgtgatccgcccgcctcggcctcccaaagtgctgggattacaggcgtgagccaccgtgcccggccgggaggtcactttaaggagggctgtccaactgcaaggagccgcaagcaggaagtgaaaccacagcacttcaaaaaaagagggagactgggacaagcgtatctaagaggctgaacatgaatccacagatcagaaatccgatggagcggatgtatcgagacacattctacgacaactttgaaaacgaacccatcctctatggtcggagctacacttggctgtgctatgaagtgaaaataaagaggggccgctcaaatctcctttgggacacaggggtctttcgaggcccggtactacccaaacgtcagtcgaatcacaggcaggaggtgtatttccggtttgagaaccacgcagaaatgtgcttcttatcttggttctgtggcaaccgactgcctgctaacaggcgcttccagatcacctggtttgtatcatggaacccctgcctgccctgtgtggtgaaggtgaccaaattcttggctgagcaccccaatgtcaccctgaccatctctgccgcccgcctctactactaccgggatagagattggcggtgggtgctcctcaggctgcataaggcaggggcccgtgtgaagatcatggactatgaagactttgcatactgctgggaaaactttgtgtgcaatgaaggtcagccattcatgccttggtacaaattcgatgacaattatgcatccctgcaccgcacgctaaaggagattctcagaaacccgatggaggcaatgtacccacacatattctacttccactttaaaaacctactgaaagcctgtggtcggaacgaaagctggctgtgcttcaccatggaagttacaaagcaccactcagctgtcttccggaagaggggcgtcttccgaaaccaggtggatcctgagacccattgtcatgcagaaaggtgcttcctctcttggttctgtgacgacatactgtctcctaacacaaactacgaggtcacctggtacacatcttggagcccttgcccagagtgtgcaggggaggtggccgagttcctggccaggcacagcaacgtgaatctcaccatcttcaccgcccgcctctgctacttctgggatacagattaccaggaggggctctgcagcctgagtcaggaaggggcctccgtgaagatcatgggctacaaagattttgtatcttgttggaaaaactttgtgtacagtgatgatgagccattcaagccttggaagggactacaaaccaactttcgacttctgaaaagaaggctacgggagattctccagtgaggggtctccctgggcctcatggtctgtctcttctagcctcctgctcatgctgcacgggcctcccctccatcctgcaccagctgtgcttttgcctggtcatcctgagcccctcctggcctcagggccattccatagtgcccccctgcctcaccacctcctccccgctctcccaggctcttcttgtagaggctctccatccacctccccagtcctgttcccccagcctgggtgcccctaacttgactcttcccatctccccagcataaccaaatcttttttttttttttttttttttgagacggagtttcactctgtcgcccagactagagtgcaatggctggatctcagctcactgcaaactctgcttactgggttcaagtgattctcctgtctcagcttctgagtagctgggattacagatgcctgccaccacgcccagctaatttttttttttttttttttttttttttgtatttttagtagtgactgggtttcaccatgttggccaggctggtcttgaactcctgacctcaggtgatctgcccatctctgtctcccaaagtgctgggattacgggcgtgagccacccggctcggccgcacaaccaaatcttattaaactcaatctagtctggccctggtgactcacgcctttcggaggcagaggtgggagaatcgcttgagcccagaagtttgagaccagcctgggccacacgacaaagccccatttctacaaaaaaaaataccaaaaaaaagccagatgtggtggcatgcacctgtagtttaagctacttgggaggatgaagtgggaggactgcttgagccggggaggtggaggctggagtaaactgagatcgcgccacagaactccagtttgagcaacagatcaagaccctgcctgaaaataaatcaataaatacactcaacctaaatggaaaaaaaaaaaaaaaaa

SEQ ID NO: 28 - protein, 386 aa, homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 29 - DNA, 9601 bp, homo sapiens
agacactgctgctccggggggctgacctggcggggagtggccgcgcagtctgctccggcgccgctttgtgcgcgcagccgctggcccctctactcccgggtctgccccccgggacacccctctgcctcgcccaagtcatgcagccctacctgcctctccactgtggacctttgggaaccgactcctcacctcgggggctcgggccttgactgtgctgggagccggtaggcgtcctccgcgacccgcccgcgcccctcgcgcccgccggggccccgggctccaaagttgtggggaccggcgcgagttggaaagtttgcccgagggctggtgcaggcttggagctgggggccgtgcgctgccctgggaatgtgacccggccagcgaccaaaaccttgtgtgactgagctgaagagcagtgcatccagattctcctcagaagtgagactttccaaaggaccaatgactctgtttcctgcgccctttcattttttcctactctgtagctatgtctcgatcccgccatgcaaggccttccagattagtcaggaaggaagatgtaaacaaaaaaaagaaaaacagccaactacgaaagacaaccaagggagccaacaaaaatgtggcatcagtcaagactttaagccctggaaaattaaagcaattaattcaagaaagagatgttaagaaaaaaacagaacctaaaccacccgtgccagtcagaagccttctgacaagagctggagcagcacgcatgaatttggataggactgaggttctttttcagaacccagagtccttaacctgcaatgggtttacaatggcgctacgaagcacctctcttagcaggcgactctcccaacccccactggtcgtagccaaatccaaaaaggttccactttctaagggtttagaaaagcaacatgattgtgattataagatactccctgctttgggagtaaagcactcagaaaatgattcggttccaatgcaagacacccaagtccttcctgatatagagactctaattggtgtacaaaatccctctttacttaaaggtaagagccaagagacaactcagttttggtcccaaagagttgaggattccaagatcaatatccctacccacagtggccctgcagctgagatccttcctgggccactggaagggacacgctgtggtgaaggactattctctgaagagacattgaatgataccagtggttccccaaaaatgtttgctcaggacacagtgtgtgctccttttccccaaagagcaacccccaaagttacctctcaaggaaaccccagcattcagttagaagagttgggttcacgagtagaatctcttaagttatctgattcttacctggatcccattaaaagtgaacatgattgctaccccacctccagtcttaataaggttatacctgacttgaaccttagaaactgcttggctcttggtgggtctacgtctcctacctctgtaataaaattcctcttggcaggctcaaaacaagcgacccttggtgctaaaccagatcatcaagaggccttcgaagctactgcaaatcaacaggaagtttctgataccacctctttcctaggacaggcctttggtgctatcccacatcaatgggaacttcctggtgctgacccagttcatggtgaggccctgggtgagaccccagatctaccagagattcctggtgctattccagtccaaggagaggtctttggtactattttagaccaacaagaaactcttggtatgagtgggagtgttgtcccagacttgcctgtcttccttcctgttcctccaaatccaattgctacctttaatgctccttccaaatggcctgagccccaaagcactgtctcatatggacttgcagtccagggtgctatacagattttgcctttgggctcaggacacactcctcaatcatcatcaaactcagagaaaaattcattacctccagtaatggctataagcaatgtagaaaatgagaagcaggttcatataagcttcctgccagctaacactcaggggttcccattagcccctgagagaggactcttccatgcttcactgggtatagcccaactctctcaggctggtcctagcaaatcagacagagggagctcccaggtcagtgtaaccagcacagttcatgttgtcaacaccacagtggtgactatgccagtgccaatggtcagtacctcctcttcttcctataccactttgctaccgactttggaaaagaagaaaagaaagcgatgtggggtctgtgaaccctgccagcagaagaccaactgtggtgaatgcacttactgcaagaacagaaagaacagccatcagatctgtaagaaaagaaaatgtgaggagctgaaaaagaaaccatctgttgttgtgcctctggaggttataaaggaaaacaagaggccccagagggaaaagaagcccaaagttttaaaggcagattttgacaacaaaccagtaaatggccccaagtcagaatccatggactacagtagatgtggtcatggggaagaacaaaaattggaattgaacccacatactgttgaaaatgtaactaaaaatgaagacagcatgacaggcatcgaggtggagaagtggacacaaaacaagaaatcacagttaactgatcacgtgaaaggagattttagtgctaatgtcccagaagctgaaaaatcgaaaaactctgaagttgacaagaaacgaaccaaatctccaaaattgtttgtacaaaccgtaagaaatggcattaaacatgtacactgtttaccagctgaaacaaatgtttcatttaaaaaattcaatattgaagaattcggcaagacattggaaaacaattcttataaattcctaaaagacactgcaaaccataaaaacgctatgagctctgttgctactgatatgagttgtgatcatctcaaggggagaagtaacgttttagtattccagcagcctggctttaactgcagttccattccacattcttcacactccatcataaatcatcatgctagtatacacaatgaaggtgatcaaccaaaaactcctgagaatataccaagtaaagaaccaaaagatggatctcccgttcaaccaagtctcttatcgttaatgaaagataggagattaacattggagcaagtggtagccatagaggccctgactcaactctcagaagccccatcagagaattcctccccatcaaagtcagagaaggatgaggaatcagagcagagaacagccagtttgcttaatagctgcaaagctatcctctacactgtaagaaaagacctccaagacccaaacttacagggagagccaccaaaacttaatcactgtccatctttggaaaaacaaagttcatgcaacacggtggttttcaatgggcaaactactaccctttccaactcacatatcaactcagctactaaccaagcatccacaaagtcacatgaatattcaaaagtcacaaattcattatctctttttataccaaaatcaaattcatccaagattgacaccaataaaagtattgctcaagggataattactcttgacaattgttccaatgatttgcatcagttgccaccaagaaataatgaagtggagtattgcaaccagttactggacagcagcaaaaaattggactcagatgatctatcatgtcaggatgcaacccatacccaaattgaggaagatgttgcaacacagttgacacaacttgcttcgataattaagatcaattatataaaaccagaggacaaaaaagttgaaagtacaccaacaagccttgtcacatgtaatgtacagcaaaaatacaatcaggagaagggcacaatacaacagaaaccaccttcaagtgtacacaataatcatggttcatcattaacaaaacaaaagaacccaacccagaaaaagacaaaatccaccccatcaagagatcggcggaaaaagaagcccacagttgtaagttatcaagaaaatgatcggcagaagtgggaaaagttgtcctatatgtatggcacaatatgcgacatttggatagcatcgaaatttcaaaattttgggcaattttgtccacatgattttcctactgtatttgggaaaatttcttcctcgaccaaaatatggaaaccactggctcaaacgaggtccattatgcaacccaaaacagtatttccaccactcactcagataaaattacagagatatcctgaatcagcagaggaaaaggtgaaggttgaaccattggattcactcagcttatttcatcttaaaacggaatccaacgggaaggcattcactgataaagcttataattctcaggtacagttaacggtgaatgccaatcagaaagcccatcctttgacccagccctcctctccacctaaccagtgtgctaacgtgatggcaggcgatgaccaaatacggtttcagcaggttgttaaggagcaactcatgcatcagagactgccaacattgcctggtatctctcatgaaacacccttaccggagtcagcactaactctcaggaatgtaaatgtagtgtgttcaggtggaattacagtggtttctaccaaaagtgaagaggaagtctgttcatccagttttggaacatcagaattttccacagtggacagtgcacagaaaaattttaatgattatgccatgaacttctttactaaccctacaaaaaacctagtgtctataactaaagattctgaactgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgtcccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgcagctgctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtctgaaggcttttctccccctcttaatgcctttgctagtgcagtgtattttttcaaggtgctgttaaaagaaagtcatgttgtcgtttactatcttcatctcacccatttcaagtctgaggtaaaaaaataataatgataacaaaacggggtgggtattcttaactgtgactatattttgacaattggtagaaggtgcacattttaagcaaaaataaaagttttatagttttaaatacataaagaaatgtttcagttaggcattaaccttgatagaatcactcagtttggtgctttaaattaagtctgtttactatgaaacaagagtcatttttagaggattttaacaggttcatgttctatgatgtaaaatcaagacacacagtgttaactctacacagcttctggtgcttaaccacatccacacagttaaaaataagctgaattattatttcatggtgccattgttccaacatcttccaatcattgctagaaaattggcatattcctttgaaataaacttatgaaatgttttctctcttaaaatatttctcctgtgtaaaataaatcattgttgttagtaatggttggaggctgttcataaattgtaaatatatattttaaaagcactttctatttttaaaagtaacttgaaataatatagtataagaatcctattgtctattgtttgtgcatatttgcatacaagagaaatcatttatccttgctgtgtagagttccatcttgttaactgcagtatgtattctaatcatgtatatggtttgtgttcttttactgtgtcctctcacattcaagtattagcaacttgcagtatataaaatagttagataatgagaagttgttaattatctctaaaattggaattaggaagcatatcaccaatactgattaacattctctttggaactaggtaagagtggtctcttcttattgaacaacctcaatttagtttcatcccacctttctcagtataatccatgagaggtgtttccaaaaggagatgagggaacaggataggtttcagaagagtcaaatgcttctaatgtctcaaggtgataaaatacaaaaactaagtagacagatatttgtactgaagtctgatacagaattagaaaaaaaaaattcttgttgaaatattttgaaaacaaattccctactatcatcacatgcctccccaaccccaagtcaaaaacaagaggaatggtactacaaacatggctttgtccattaagagctaattcatttgtttatcttagcatactagatttgggaaaatgataactcatcttttctgataattgcctatgttctaggtaacaggaaaacaggcattaagtttattttagtcttcccattttcttcctattactttattgactcattttattgcaaaacaaaaaggattacccaaacaacatgtttcgaacaaggagaattttcaatgaaatacttgattctgttaaaatgcagaggtgctataacattcaaagtgtcagattccttgggagtatggaaaacctaatggtgcttctcccttggaaatgccataggaagcccacaaccgctaacacttacaattttggtgcaaaagcaaacagttccagcaggctctctaaagaaaaactcattgtaacttattaaaataatatctggtgcaaagtatctgttttgagcttttgactaatccaagtaaaggaatatgaagggattgtaaaaaacaaaatgtccattgatagaccatcgtgtacaagtagatttctgcttgttgaatatgtaaaatagggtaattcattgacttgttttagtattttgtgtgccttagatttccgttttaagacatgtatatttttgtgagcctaaggtttcttatatacatataagtatataaataagtgattgtttattgcttcagctgcttcaacaagatatttactagtattagactatcaggaatacacccttgcgagattatgttttagattttaggccttagctcccactagaaattatttcttcaccagatttaatggataaagttttatggctctttatgcatccactcatctactcattcttcgagtctacacttattgaatgcctgcaaaatctaagtatcacttttatttttctttggatcaccacctatgacatagtaaacttgaagaataaaaactaccctcagaaatatttttaaaagaagtagcaaattatcttcagtataatccatggtaatgtatgcagtaattcaaattgatctctctctcaataggtttcttaacaatctaaacttgaaacatcaatgttaatttttggaactattgggatttgtgacgcttgttgcagtttaccaaaacaagtatttgaaaatatatagtatcaactgaaatgtttccattccgttgttgtagttaacatcatgaatggacttcttaagctgattaccccactgtgggaaccaaattggattcctactttgttggactctctttcctgattttaacaatttaccatcccattctctgccctgtgattttttttaaaagcttattcaatgttctgcagcattgtgattgtatgctggctacactgcttttagaatgctctttctcatgaagcaaggaaataaatttgtttgaaatgacattttctctcaaaaaaaaaaaaaaaaaa

SEQ ID NO: 30 - protein, 2136 aa, homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 31 - DNA, 9677 bp, homo sapiens
gcggccgccccgagacgccggccccgctgagtgatgagaacagacgtcaaactgccttatgaatattgatgcggaggctaggctgctttcgtagagaagcagaaggaagcaagatggctgccctttaggatttgttagaaaggagacccgactgcaactgctggattgctgcaaggctgagggacgagaacgaggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtagagcaaattattgaaaaagatgaaggtcctttttatacccatctaggagcaggtcctaatgtggcagctattagagaaatcatggaagaaaggtttggacagaagggtaaagctattaggattgaaagagtcatctatactggtaaagaaggcaaaagttctcagggatgtcctattgctaagtgggtggttcgcagaagcagcagtgaagagaagctactgtgtttggtgcgggagcgagctggccacacctgtgaggctgcagtgattgtgattctcatcctggtgtgggaaggaatcccgctgtctctggctgacaaactctactcggagcttaccgagacgctgaggaaatacggcacgctcaccaatcgccggtgtgccttgaatgaagagagaacttgcgcctgtcaggggctggatccagaaacctgtggtgcctccttctcttttggttgttcatggagcatgtactacaatggatgtaagtttgccagaagcaagatcccaaggaagtttaagctgcttggggatgacccaaaagaggaagagaaactggagtctcatttgcaaaacctgtccactcttatggcaccaacatataagaaacttgcacctgatgcatataataatcagattgaatatgaacacagagcaccagagtgccgtctgggtctgaaggaaggccgtccattctcaggggtcactgcatgtttggacttctgtgctcatgcccacagagacttgcacaacatgcagaatggcagcacattggtatgcactctcactagagaagacaatcgagaatttggaggaaaacctgaggatgagcagcttcacgttctgcctttatacaaagtctctgacgtggatgagtttgggagtgtggaagctcaggaggagaaaaaacggagtggtgccattcaggtactgagttcttttcggcgaaaagtcaggatgttagcagagccagtcaagacttgccgacaaaggaaactagaagccaagaaagctgcagctgaaaagctttcctccctggagaacagctcaaataaaaatgaaaaggaaaagtcagccccatcacgtacaaaacaaactgaaaacgcaagccaggctaaacagttggcagaacttttgcgactttcaggaccagtcatgcagcagtcccagcagccccagcctctacagaagcagccaccacagccccagcagcagcagagaccccagcagcagcagccacatcaccctcagacagagtctgtcaactcttattctgcttctggatccaccaatccatacatgagacggcccaatccagttagtccttatccaaactcttcacacacttcagatatctatggaagcaccagccctatgaacttctattccacctcatctcaagctgcaggttcatatttgaattcttctaatcccatgaacccttaccctgggcttttgaatcagaatacccaatatccatcatatcaatgcaatggaaacctatcagtggacaactgctccccatatctgggttcctattctccccagtctcagccgatggatctgtataggtatccaagccaagaccctctgtctaagctcagtctaccacccatccatacactttaccagccaaggtttggaaatagccagagttttacatctaaatacttaggttatggaaaccaaaatatgcagggagatggtttcagcagttgtaccattagaccaaatgtacatcatgtagggaaattgcctccttatcccactcatgagatggatggccacttcatgggagccacctctagattaccacccaatctgagcaatccaaacatggactataaaaatggtgaacatcattcaccttctcacataatccataactacagtgcagctccgggcatgttcaacagctctcttcatgccctgcatctccaaaacaaggagaatgacatgctttcccacacagctaatgggttatcaaagatgcttccagctcttaaccatgatagaactgcttgtgtccaaggaggcttacacaaattaagtgatgctaatggtcaggaaaagcagccattggcactagtccagggtgtggcttctggtgcagaggacaacgatgaggtctggtcagacagcgagcagagctttctggatcctgacattgggggagtggccgtggctccaactcatgggtcaattctcattgagtgtgcaaagcgtgagctgcatgccacaacccctttaaagaatcccaataggaatcaccccaccaggatctccctcgtcttttaccagcataagagcatgaatgagccaaaacatggcttggctctttgggaagccaaaatggctgaaaaagcccgtgagaaagaggaagagtgtgaaaagtatggcccagactatgtgcctcagaaatcccatggcaaaaaagtgaaacgggagcctgctgagccacatgaaacttcagagcccacttacctgcgtttcatcaagtctcttgccgaaaggaccatgtccgtgaccacagactccacagtaactacatctccatatgccttcactcgggtcacagggccttacaacagatatatatgatatcacccccttttgttggttacctcacttgaaaagaccacaaccaacctgtcagtagtatagttctcatgacgtgggcagtggggaaaggtcacagtattcatgacaaatgtggtgggaaaaacctcagctcaccagcaacaaaagaggttatcttaccatagcacttaattttcactggctcccaagtggtcacagatggcatctaggaaaagaccaaagcattctatgcaaaaagaaggtggggaagaaagtgttccgcaatttacatttttaaacactggttctattattggacgagatgatatgtaaatgtgatcccccccccccgcttacaactctacacatctgtgaccacttttaataatatcaagtttgcatagtcatggaacacaaatcaaacaagtactgtagtattacagtgacaggaatcttaaaataccatctggtgctgaatatatgatgtactgaaatactggaattatggctttttgaaatgcagtttttactgtaatcttaacttttatttatcaaaatagctacaggaaacatgaatagcaggaaaacactgaatttgtttggatgttctaagaaatggtgctaagaaaatggtgtctttaatagctaaaaatttaatgcctttatatcatcaagatgctatcagtgtactccagtgcccttgaataataggggtaccttttcattcaagtttttatcataattacctattcttacacaagcttagtttttaaaatgtggacattttaaaggcctctggattttgctcatccagtgaagtccttgtaggacaataaacgtatatatgtacatatatacacaaacatgtatatgtgcacacacatgtatatgtataaatattttaaatggtgttttagaagcactttgtctacctaagctttgacaacttgaacaatgctaaggtactgagatgtttaaaaaacaagtttactttcattttagaatgcaaagttgatttttttaaggaaacaaagaaagcttttaaaatatttttgcttttagccatgcatctgctgatgagcaattgtgtccatttttaacacagccagttaaatccaccatggggcttactggattcaagggaatacgttagtccacaaaacatgttttctggtgctcatctcacatgctatactgtaaaacagttttatacaaaattgtatgacaagttcattgctcaaaaatgtacagttttaagaattttctattaactgcaggtaataattagctgcatgctgcagactcaacaaagctagttcactgaagcctatgctattttatggatcataggctcttcagagaactgaatggcagtctgcctttgtgttgataattatgtacattgtgacgttgtcatttcttagcttaagtgtcctctttaacaagaggattgagcagactgatgcctgcataagatgaataaacagggttagttccatgtgaatctgtcagttaaaaagaaacaaaaacaggcagctggtttgctgtggtggttttaaatcattaatttgtataaagaagtgaaagagttgtatagtaaattaaattgtaaacaaaacttttttaatgcaatgctttagtattttagtactgtaaaaaaattaaatatatacatatatatatatatatatatatatatatatatgagtttgaagcagaattcacatcatgatggtgctactcagcctgctacaaatatatcataatgtgagctaagaattcattaaatgtttgagtgatgttcctacttgtcatatacctcaacactagtttggcaataggatattgaactgagagtgaaagcattgtgtaccatcatttttttccaagtccttttttttattgttaaaaaaaaaagcataccttttttcaatacttgatttcttagcaagtataacttgaacttcaacctttttgttctaaaaattcagggatatttcagctcatgctctccctatgccaacatgtcacctgtgtttatgtaaaattgttgtaggttaataaatatattctttgtcagggatttaacccttttattttgaatcccttctattttacttgtacatgtgctgatgtaactaaaactaattttgtaaatctgttggctctttttattgtaaagaaaagcattttaaaagtttgaggaatcttttgactgtttcaagcaggaaaaaaaaattacatgaaaatagaatgcactgagttgataaagggaaaaattgtaaggcaggagtttggcaagtggctgttggccagagacttacttgtaactctctaaatgaagtttttttgatcctgtaatcactgaaggtacatactccatgtggacttcccttaaacaggcaaacacctacaggtatggtgtgcaacagattgtacaattacattttggcctaaatacatttttgcttactagtatttaaaataaattcttaatcagaggaggcctttgggttttattggtcaaatctttgtaagctggcttttgtctttttaaaaaatttcttgaatttgtggttgtgtccaatttgcaaacatttccaaaaatgtttgctttgcttacaaaccacatgattttaatgttttttgtataccataatatctagccccaaacatttgattactacatgtgcattggtgattttgatcatccattcttaatatttgatttctgtgtcacctactgtcatttgttaaactgctggccaacaagaacaggaagtatagtttggggggttggggagagtttacataaggaagagaagaaattgagtggcatattgtaaatatcagatctataattgtaaatataaaacctgcctcagttagaatgaatggaaagcagatctacaatttgctaatataggaatatcaggttgactatatagccatacttgaaaatgcttctgagtggtgtcaactttacttgaatgaatttttcatcttgattgacgcacagtgatgtacagttcacttctgaagctagtggttaacttgtgtaggaaacttttgcagtttgacactaagataacttctgtgtgcatttttctatgcttttttaaaaactagtttcatttcattttcatgagatgtttggtttataagatctgaggatggttataaatactgtaagtattgtaatgttatgaatgcaggttatttgaaagctgtttattattatatcattcctgataatgctatgtgagtgtttttaataaaatttatatttatttaatgcactct

SEQ ID NO: 32 - protein, 2002 aa, homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 33 - DNA, 9215 bp, homo sapiens
gcggccgccccgagacgccggccccgctgagtgatgagaacagacgtcaaactgccttatgaatattgatgcggaggctaggctgctttcgtagagaagcagaaggaagcaagatggctgccctttaggatttgttagaaaggagacccgactgcaactgctggattgctgcaaggctgagggacgagaacgaggctggcaaacattcagcagcacaccctctcaagattgtttacttgcctttgctcctgttgagttacaacgcttggaagcaggagatgggctcagcagcagccaataggacatgatccaggaagagcagtaagggactgagctgctgaattcaactagagggcagccttgtggatggccccgaagcaagcctgatggaacaggatagaaccaaccatgttgagggcaacagactaagtccattcctgataccatcacctcccatttgccagacagaacctctggctacaaagctccagaatggaagcccactgcctgagagagctcatccagaagtaaatggagacaccaagtggcactctttcaaaagttattatggaataccctgtatgaagggaagccagaatagtcgtgtgagtcctgactttacacaagaaagtagagggtattccaagtgtttgcaaaatggaggaataaaacgcacagttagtgaaccttctctctctgggctccttcagatcaagaaattgaaacaagaccaaaaggctaatggagaaagacgtaacttcggggtaagccaagaaagaaatccaggtgaaagcagtcaaccaaatgtctccgatttgagtgataagaaagaatctgtgagttctgtagcccaagaaaatgcagttaaagatttcaccagtttttcaacacataactgcagtgggcctgaaaatccagagcttcagattctgaatgagcaggaggggaaaagtgctaattaccatgacaagaacattgtattacttaaaaacaaggcagtgctaatgcctaatggtgctacagtttctgcctcttccgtggaacacacacatggtgaactcctggaaaaaacactgtctcaatattatccagattgtgtttccattgcggtgcagaaaaccacatctcacataaatgccattaacagtcaggctactaatgagttgtcctgtgagatcactcacccatcgcatacctcagggcagatcaattccgcacagacctctaactctgagctgcctccaaagccagctgcagtggtgagtgaggcctgtgatgctgatgatgctgataatgccagtaaactagctgcaatgctaaatacctgttcctttcagaaaccagaacaactacaacaacaaaaatcagtttttgagatatgcccatctcctgcagaaaataacatccagggaaccacaaagctagcgtctggtgaagaattctgttcaggttccagcagcaatttgcaagctcctggtggcagctctgaacggtatttaaaacaaaatgaaatgaatggtgcttacttcaagcaaagctcagtgttcactaaggattccttttctgccactaccacaccaccaccaccatcacaattgcttctttctccccctcctcctcttccacaggttcctcagcttccttcagaaggaaaaagcactctgaatggtggagttttagaagaacaccaccactaccccaaccaaagtaacacaacacttttaagggaagtgaaaatagagggtaaacctgaggcaccaccttcccagagtcctaatccatctacacatgtatgcagcccttctccgatgctttctgaaaggcctcagaataattgtgtgaacaggaatgacatacagactgcagggacaatgactgttccattgtgttctgagaaaacaagaccaatgtcagaacacctcaagcataacccaccaatttttggtagcagtggagagctacaggacaactgccagcagttgatgagaaacaaagagcaagagattctgaagggtcgagacaaggagcaaacacgagatcttgtgcccccaacacagcactatctgaaaccaggatggattgaattgaaggcccctcgttttcaccaagcggaatcccatctaaaacgtaatgaggcatcactgccatcaattcttcagtatcaacccaatctctccaatcaaatgacctccaaacaatacactggaaattccaacatgcctggggggctcccaaggcaagcttacacccagaaaacaacacagctggagcacaagtcacaaatgtaccaagttgaaatgaatcaagggcagtcccaaggtacagtggaccaacatctccagttccaaaaaccctcacaccaggtgcacttctccaaaacagaccatttaccaaaagctcatgtgcagtcactgtgtggcactagatttcattttcaacaaagagcagattcccaaactgaaaaacttatgtccccagtgttgaaacagcacttgaatcaacaggcttcagagactgagccattttcaaactcacaccttttgcaacataagcctcataaacaggcagcacaaacacaaccatcccagagttcacatctccctcaaaaccagcaacagcagcaaaaattacaaataaagaataaagaggaaatactccagacttttcctcacccccaaagcaacaatgatcagcaaagagaaggatcattctttggccagactaaagtggaagaatgttttcatggtgaaaatcagtattcaaaatcaagcgagttcgagactcataatgtccaaatgggactggaggaagtacagaatataaatcgtagaaattccccttatagtcagaccatgaaatcaagtgcatgcaaaatacaggtttcttgttcaaacaatacacacctagtttcagagaataaagaacagactacacatcctgaactttttgcaggaaacaagacccaaaacttgcatcacatgcaatattttccaaataatgtgatcccaaagcaagatcttcttcacaggtgctttcaagaacaggagcagaagtcacaacaagcttcagttctacagggatataaaaatagaaaccaagatatgtctggtcaacaagctgcgcaacttgctcagcaaaggtacttgatacataaccatgcaaatgtttttcctgtgcctgaccagggaggaagtcacactcagacccctccccagaaggacactcaaaagcatgctgctctaaggtggcatctcttacagaagcaagaacagcagcaaacacagcaaccccaaactgagtcttgccatagtcagatgcacaggccaattaaggtggaacctggatgcaagccacatgcctgtatgcacacagcaccaccagaaaacaaaacatggaaaaaggtaactaagcaagagaatccacctgcaagctgtgataatgtgcagcaaaagagcatcattgagaccatggagcagcatctgaagcagtttcacgccaagtcgttatttgaccataaggctcttactctcaaatcacagaagcaagtaaaagttgaaatgtcagggccagtcacagttttgactagacaaaccactgctgcagaacttgatagccacaccccagctttagagcagcaaacaacttcttcagaaaagacaccaaccaaaagaacagctgcttctgttctcaataattttatagagtcaccttccaaattactagatactcctataaaaaatttattggatacacctgtcaagactcaatatgatttcccatcttgcagatgtgtaggtaagtgccagaaatgtactgagacacatggcgtttatccagaattagcaaatttatcttcagatatgggattttccttctttttttaaatcttgagtctggcagcaatttgtaaaggctcataaaaatctgaagcttacattttttgtcaagttaccgatgcttgtgtcttgtgaaagagaacttcacttacatgcagtttttccaaaagaattaaataatcgtgcatgtttatttttccctctcttcagatcctgtaaaatttgaatgtatctgttttagatcaattcgcctatttagctctttgtatattatctcctggagagacagctaggcagcaaaaaaacaatctattaaaatgagaaaataacgaccataggcagtctaatgtacgaactttaaatattttttaattcaaggtaaaatatattagtttcacaagatttctggctaatagggaaattattatcttcagtcttcatgagttgggggaaatgataatgctgacactcttagtgctcctaaagtttccttttctccatttatacatttggaatgttgtgatttatattcattttgattcccttttctctaaaatttcatctttttgattaaaaaatatgatacaggcatacctcagagatattgtgggtttggctccataccacaataaaatgaatattacaataaagcaagttgtaaggactttttggtttctcactgtatgtaaaagttatttatatactatactgtaacatactaagtgtgcaatagcattgtgtctaaaaaatatatactttaaaaataatttattgttaaaaaaatgccaacaattatctgggcctttagtgagtgctaatctttttgctggtggagggtcgtgcttcagtattgatcgctgtggactgatcatggtggtagttgctgaaggttgctgggatggctgtgtgtgtggcaatttcttaaaataagacaacagtgaagtgctgtatcaattgatttttccattcacaaaagatttctctgtagcatgcaatgctgtttgatagcatttaacccacagcagaatttctttgaaaattggactcagtcctctcaaactgtgctgctgctttatcaactaagtttttgtaattttctgaatcctttgttgtcatttcagcagtttacagcatcttcattggaagtatattccatctcaaacattctttgttcatccataagaagcaacttcttatcaagttttttcatgacattgcagtaactcagccccatcttcaggctctacttctaattctggttctcttgctacatctccctcatctgcagtgacctctccacggaagtcttgaactcctcaaagtaatccatgagggttggaatcaacttctaaactcctgttaatgttgatatattgaccccctcccatgaattatgaatgttcttaataacttctaaatggtgatacctttccagaaggctttcaatgtactttgcccggatccatcagaagactatcttggcagctgtagactaacaatatatttcttaaatgataagacttgaaagtcaaaagtactccttaatccataggctgcagaatcaatgttgtattaacaggcacgaaaacagcattaatcttgtgcatctccatcggagctcttgggtgactaggtgccttgagcagtaatattttgaaaggaggttttggttttgttttttgttttttttttttgttttttagcagtaagtctcaacactgggcttaaaatattcagtaaactatgttgtaaaaagatgtgttatcatccagactttgttgttccattactctacacaagcagggtacacttagcataattcttaagggccttggaattttcagaatggtaaatgagtatgggcttcaacttaaaatcatcaactgcattagcctgtaacaagagagtcagcctgtcctttgaagcaaggcattgacttctatctatgaaagtcttagatggcaccttgtttcaatagtaggctgtttagtacagccaccttcatcagtgatcttagctagatcttctgcataacttgctgcagcttctacatcagcacttgctgcctcaccttgtccttttatgttatagagacagctgcgcttcttaaactttataaaccaacttctgctagcttccaacttctcttctgcagcttcctcattctcttcatagaactgaagggagtcaaggccttgctctggattaagctttggcttaaggaatgttgtggctgacgtgatcttctatccagaccactaaagcgctctccatatcagcaataaggccgttttgctttcttacctttcatgtgttcactggagtaatttccttcaagaatttttcctttacattcacaacttggctaactggcatgcaaggcctagctttcagcctgtcttggcttttgacatgccttcctcacttagctcgtcatatctagcttttgatttaaagtggcaggcatacaactcttcctttcacttgaacacttagaggccactgtagggttattaattggcctaatttcaatattgttgtgttttagggaatagagaggcccagggagagggagagagcccaaacggctggttgatagagcaggcagaatgcacacaacatttatcagattatgtttgcaccatttaccagattatgggtacggtttgtggcaccccccaaaaattagaatagtaacatcaaagatcactgatcacagatcgccataacataaataataataaactttaaaatactgtgagaattaccaaaatgtgatacagagacatgaagtgagcacatgctgttgaaaaaaatgacactgatagacatacttaacacgtgggattgccacaaaccttcagtttgtaaaagtcacagtaactgtgactcacaaaagaacaaagcacaataaaacgaggtatgcctgtatttttaaaaaaagctttttgttaaaattcaggatatgtaataggtctgtaggaatagtgaaatatttttgctgatggatgtagatatatacgtggatagagatgaagatcttaattatagctatgcagcatagatttagtcaaagacatttgaaaagacaaatgttaaattagtgtggctaatgacctacccgtgccatgttttccctcttgcaatgagataccccacactgtgtagaaggatggagggaggactcctactgtccctctttgcgtgtggttattaagttgcctcactgggctaaaacaccacacatctcatagataatatttggtaagttgtaatcgtcttcactcttctcttatcacccacccctatcttcccacttttccatctttgttggtttgcaacagccccttctttttgcctgactctccaggattttctctcatcataaattgttctaaagtacatactaatatgggtctggattgactattcttatttgcaaaacagcaattaaatgttatagggaagtaggaagaaaaaggggtatccttgacaataaaccaagcaatattctgggggtgggatagagcaggaaattttatttttaatcttttaaaatccaagtaataggtaggcttccagttagctttaaatgttttttttttccagctcaaaaaattggattgtagttgatactacatataatacattctaattccctcactgtattctttgtttagtttcatttatttggtttaaaataattttttatcccatatctgaaatgtaatatatttttatccaacaaccagcatgtacatatacttaattatgtggcacattttctaatagatcagtccatcaatctactcattttaaagaaaaaaaaattttaaagtcacttttagagcccttaatgtgtagttgggggttaagctttgtggatgtagcctttatatttagtataattgaggtctaaaataataatcttctattatctcaacagagcaaattattgaaaaagatgaaggtcctttttatacccatctaggagcaggtcctaatgtggcagctattagagaaatcatggaagaaaggtaattaacgcaaaggcacagggcagattaacgtttatccttttgtatatgtcagaatttttccagccttcacacacaaagcagtaaacaattgtaaattgagtaattattagtaggcttagctattctagggttgccaacactacacactgtgctattcaccagagagtcacaatatttgacaggactaatagtctgctagctggcacaggctgcccactttgcgatggatgccagaaaacccaggcatgaacaggaatcggccagccaggctgccagccacaaggtactggcacaggctccaacgagaggtcccactctggctttcccacctgataataaagtgtcaaagcagaaagactggtaaagtgtggtataagaaaagaaccactgaattaaattcacctagtgttgcaaatgagtacttatctctaagttttcttttaccataaaaagagagcaagtgtgatatgttgaatagaaagagaaacatactatttacagctgcctttttttttttttttcgctatcaatcacaggtatacaagtacttgcctttactcctgcatgtagaagactcttatgagcgagataatgcagagaaggcctttcatataaatttatacagctctgagctgttcttcttctagggtgccttttcattaagaggtaggcagtattattattaaagtacttaggatacattggggcagctaggacatattcagtatcattcttgctccatttccaaattattcatttctaaattagcatgtagaagttcactaaataatcatctagtggcctggcagaaatagtgaatttccctaagtgccttttttttgttgtttttttgttttgttttttaaacaagcagtaggtggtgctttggtcataagggaagatatagtctatttctaggactattccatattttccatgtggctggatactaactatttgccagcctccttttctaaattgtgagacattcttggaggaacagttctaactaaaatctattatgactccccaagttttaaaatagctaaatttagtaagggaaaaaatagtttatgttttagaagactgaacttagcaaactaacctgaattttgtgctttgtgaaattttatatcgaaatgagctttcccattttcacccacatgtaatttacaaaatagttcattacaattatctgtacattttgatattgaggaaaaacaaggcttaaaaaccattatccagtttgcttggcgtagacctgtttaaaaaataataaaccgttcatttctcaggatgtggtcatagaataaagttatgctcaaatgttcaaatattt

SEQ ID NO: 34 - protein, 1165 aa, homo sapiens
MEQDRTNHVEGNRLSPFLIPSPPICQTEPLATKLQNGSPLPERAHPEVNGDTKWHSFKSYYGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVGKCQKCTETHGVYPELANLSSDMGFSFFF

SEQ ID NO: 35 - DNA, 2470 bp, homo sapiens
ggcggctgtagccgagggggcggccggaaagcagcggcggcgtctggggcgctttcgcaacattcagacctcggttgcagcccggtgccgtgagctgaagaggtttcacatcttactccgccccacaccctgggcgttgcggcgctgggctcgttgctgcagccggaccctgctcgatgggcacgactgggctggagagtctgagtctgggggaccgcggagctgcccccaccgtcacctctagtgagcgcctagtcccagacccgccgaatgacctccgcaaagaagatgttgctatggaattggaaagagtgggagaagatgaggaacaaatgatgataaaaagaagcagtgaatgtaatcccttgctacaagaacccatcgcttctgctcagtttggtgctactgcaggaacagaatgccgtaagtctgtcccatgtggatgggaaagagttgtgaagcaaaggttatttgggaagacagcaggaagatttgatgtgtactttatcagcccacaaggactgaagttcagatccaaaagttcacttgctaattatcttcacaaaaatggagagacttctcttaagccagaagattttgattttactgtactttctaaaaggggtatcaagtcaagatataaagactgcagcatggcagccctgacatcccatctacaaaaccaaagtaacaattcaaactggaacctcaggacccgaagcaagtgcaaaaaggatgtgtttatgccgccaagtagtagttcagagttgcaggagagcagaggactctctaactttacttccactcatttgcttttgaaagaagatgagggtgttgatgatgttaacttcagaaaggttagaaagcccaaaggaaaggtgactattttgaaaggaatcccaattaagaaaactaaaaaaggatgtaggaagagctgttcaggttttgttcaaagtgatagcaaaagagaatctgtgtgtaataaagcagatgctgaaagtgaacctgttgcacaaaaaagtcagcttgatagaactgtctgcatttctgatgctggagcatgtggtgagaccctcagtgtgaccagtgaagaaaacagccttgtaaaaaaaaaagaaagatcattgagttcaggatcaaatttttgttctgaacaaaaaacttctggcatcataaacaaattttgttcagccaaagactcagaacacaacgagaagtatgaggatacctttttagaatctgaagaaatcggaacaaaagtagaagttgtggaaaggaaagaacatttgcatactgacattttaaaacgtggctctgaaatggacaacaactgctcaccaaccaggaaagacttcactggtgagaaaatatttcaagaagataccatcccacgaacacagatagaaagaaggaaaacaagcctgtatttttccagcaaatataacaaagaagctcttagccccccacgacgtaaagcctttaagaaatggacacctcctcggtcaccttttaatctcgttcaagaaacactttttcatgatccatggaagcttctcatcgctactatatttctcaatcggacctcaggcaaaatggcaatacctgtgctttggaagtttctggagaagtatccttcagctgaggtagcaagaaccgcagactggagagatgtgtcagaacttcttaaacctcttggtctctacgatcttcgggcaaaaaccattgtcaagttctcagatgaatacctgacaaagcagtggaagtatccaattgagcttcatgggattggtaaatatggcaacgactcttaccgaattttttgtgtcaatgagtggaagcaggtgcaccctgaagaccacaaattaaataaatatcatgactggctttgggaaaatcatgaaaaattaagtctatcttaaactctgcagctttcaagctcatctgttatgcatagctttgcacttcaaaaaagcttaattaagtacaaccaaccacctttccagccatagagattttaattagcccaactagaagcctagtgtgtgtgctttcttaatgtgtgtgccaatggtggatctttgctactgaatgtgtttgaacatgttttgagatttttttaaaataaattattatttgacaacaatccaaaaaaaatacggcttttccaatgatgaaatataatcagaagatgaaaaatagttttaaactatcaataatacaaagcaaatttctatcagccttgctaaagctaggggcccactaaatatttttatcggctaggcgtggtggtgcatgcctgtaatctcggaaggctgaggcaggaggatcatttgagctcatgagggcccaggaggtcaaggcttcagtgagccatgatcatgccactgcactccagtctggatgacagagagagaccctgtctcaaaaaatatatatttaaaaaataaaaataaaagctgaccccaaagac

SEQ ID NO: 36 - protein, 580 aa, homo sapiens
MGTTGLESLSLGDRGAAPTVTSSERLVPDPPNDLRKEDVAMELERVGEDEEQMMIKRSSECNPLLQEPIASAQFGATAGTECRKSVPCGWERVVKQRLFGKTAGRFDVYFISPQGLKFRSKSSLANYLHKNGETSLKPEDFDFTVLSKRGIKSRYKDCSMAALTSHLQNQSNNSNWNLRTRSKCKKDVFMPPSSSSELQESRGLSNFTSTHLLLKEDEGVDDVNFRKVRKPKGKVTILKGIPIKKTKKGCRKSCSGFVQSDSKRESVCNKADAESEPVAQKSQLDRTVCISDAGACGETLSVTSEENSLVKKKERSLSSGSNFCSEQKTSGIINKFCSAKDSEHNEKYEDTFLESEEIGTKVEVVERKEHLHTDILKRGSEMDNNCSPTRKDFTGEKIFQEDTIPRTQIERRKTSLYFSSKYNKEALSPPRRKAFKKWTPPRSPFNLVQETLFHDPWKLLIATIFLNRTSGKMAIPVLWKFLEKYPSAEVARTADWRDVSELLKPLGLYDLRAKTIVKFSDEYLTKQWKYPIELHGIGKYGNDSYRIFCVNEWKQVHPEDHKLNKYHDWLWENHEKLSLS

SEQ ID NO: 37 - DNA, 3251 bp, homo sapiens
gatttggctccgaggaggcggaagtgcagcacagaaagggggtccgtgggggacggtagaagcctggaggaggagcttgagtccagccactgtctgggtactgccagccatcgggcccaggtctctggggttgtcttaccgcagtgagtaccacgcggtactacagagaccggctgcccgtgtgcccggcaggtggagccgcccgcatcagcggcctcggggaatggaagcggagaacgcgggcagctattcccttcagcaagctcaagctttttatacgtttccatttcaacaactgatggctgaagctcctaatatggcagttgtgaatgaacagcaaatgccagaagaagttccagccccagctcctgctcaggaaccagtgcaagaggctccaaaaggaagaaaaagaaaacccagaacaacagaaccaaaacaaccagtggaacccaaaaaacctgttgagtcaaaaaaatctggcaagtctgcaaaatcaaaagaaaaacaagaaaaaattacagacacatttaaagtaaaaagaaaagtagaccgttttaatggtgtttcagaagctgaacttctgaccaagactctccccgatattttgaccttcaatctggacattgtcattattggcataaacccgggactaatggctgcttacaaagggcatcattaccctggacctggaaaccatttttggaagtgtttgtttatgtcagggctcagtgaggtccagctgaaccatatggatgatcacactctaccagggaagtatggtattggatttaccaacatggtggaaaggaccacgcccggcagcaaagatctctccagtaaagaatttcgtgaaggaggacgtattctagtacagaaattacagaaatatcagccacgaatagcagtgtttaatggaaaatgtatttatgaaatttttagtaaagaagtttttggagtaaaggttaagaacttggaatttgggcttcagccccataagattccagacacagaaactctctgctatgttatgccatcatccagtgcaagatgtgctcagtttcctcgagcccaagacaaagttcattactacataaaactgaaggacttaagagatcagttgaaaggcattgaacgaaatatggacgttcaagaggtgcaatatacatttgacctacagcttgcccaagaggatgcaaagaagatggctgttaaggaagaaaaatatgatccaggttatgaggcagcatatggtggtgcttacggagaaaatccatgcagcagtgaaccttgtggcttctcttcaaatgggctaattgagagcgtggagttaagaggagaatcagctttcagtggcattcctaatgggcagtggatgacccagtcatttacagaccaaattccttcctttagtaatcactgtggaacacaagaacaggaagaagaaagccatgcttaagaatggtgcttctcagctctgcttaaatgctgcagttttaatgcagttgtcaacaagtagaacctcagtttgctaactgaagtgttttattagtattttactctagtggtgtaattgtaatgtagaacagttgtgtggtagtgtgaaccgtatgaacctaagtagtttggaagaaaaagtagggtttttgtatactagcttttgtatttgaattaattatcattccagctttttatatactatatttcatttatgaagaaattgattttcttttgggagtcacttttaatctgtaattttaaaatacaagtctgaatatttatagttgattcttaactgcataaacctagatataccattatcccttttatacctaagaagggcatgctaataattaccactgtcaaagaggcaaaggtgttgatttttgtatatgaagttaagcctcagtggagtctcatttgttagtttttagtggtaactaagggtaaactcagggttccctgagctatatgcacactcagacctctttgctttaccagtggtgtttgtgagttgctcagtagtaaaaactggcccttacctgacagagccctggctttgacctgctcagccctgtgtgttaatcctctagtagccaattaactactctggggtggcaggttccagagaatgcagtagaccttttgccactcatctgtgttttacttgagacatgtaaatatgatagggaaggaactgaatttctccattcatatttataaccattctagttttatcttccttggctttaagagtgtgccatggaaagtgataagaaatgaacttctaggctaagcaaaaagatgctggagatatttgatactctcatttaaactggtgctttatgtacatgagatgtactaaaataagtaatatagaatttttcttgctaggtaaatccagtaagccaataattttaaagattctttatctgcatcattgctgtttgttactataaattaaatgaacctcatggaaaggttgaggtgtatacctttgtgattttctaatgagttttccatggtgctacaaataatccagactaccaggtctggtagatattaaagctgggtactaagaaatgttatttgcatcctctcagttactcctgaatattctgatttcatacgtacccagggagcatgctgttttgtcaatcaatataaaatatttatgaggtctcccccacccccaggaggttatatgattgctcttctctttataataagagaaacaaattcttattgtgaatcttaacatgctttttagctgtggctatgatggattttattttttcctaggtcaagctgtgtaaaagtcatttatgttatttaaatgatgtactgtactgctgtttacatggacgttttgtgcgggtgctttgaagtgccttgcatcagggattaggagcaattaaattattttttcacgggactgtgtaaagcatgtaactaggtattgctttggtatataactattgtagctttacaagagattgttttatttgaatggggaaaataccctttaaattatgacggacatccactagagatgggtttgaggattttccaagcgtgtaataatgatgtttttcctaacatgacagatgagtagtaaatgttgatatatcctatacatgacagtgtgagactttttcattaaataatattgaaagattttaaaattcatttgaaagtctgatggcttttacaataaaagatattaagaattgttatccttaacttaaaaaaaa

SEQ ID NO: 38 - protein, 410 aa, homo sapiens
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 39 - DNA, 1355 bp, homo sapiens
cagtggctggtaggcagtggctgggaggcagcggcccaattagtgtcgtgcggcccgtggcgaggcgaggtccggggagcgagcgagcaagcaaggcgggaggggtggccggagctgcggcggctggcacaggaggaggagcccgggcgggcgaggggcggccggagagcgccagggcctgagctgccggagcggcgcctgtgagtgagtgcagaaagcaggcgcccgcgcgctagccgtggcaggagcagcccgcacgccgcgctctctccctgggcgacctgcagtttgcaatatgactttggaggaattctcggctggagagcagaagaccgaaaggatggataaggtgggggatgccctggaggaagtgctcagcaaagccctgagtcagcgcacgatcactgtcggggtgtacgaagcggccaagctgctcaacgtcgaccccgataacgtggtgttgtgcctgctggcggcggacgaggacgacgacagagatgtggctctgcagatccacttcaccctgatccaggcgttttgctgcgagaacgacatcaacatcctgcgcgtcagcaacccgggccggctggcggagctcctgctcttggagaccgacgctggccccgcggcgagcgagggcgccgagcagcccccggacctgcactgcgtgctggtgacgaatccacattcatctcaatggaaggatcctgccttaagtcaacttatttgtttttgccgggaaagtcgctacatggatcaatgggttccagtgattaatctccctgaacggtgatggcatctgaatgaaaataactgaaccaaattgcactgaagtttttgaaatacctttgtagttactcaagcagttactccctacactgatgcaaggattacagaaactgatgccaaggggctgagtgagttcaactacatgttctgggggcccggagatagatgactttgcagatggaaagaggtgaaaatgaagaaggaagctgtgttgaaacagaaaaataagtcaaaaggaacaaaaattacaaagaaccatgcaggaaggaaaactatgtattaatttagaatggttgagttacattaaaataaaccaaatatgttaaagtttaagtgtgcagccatagtttgggtatttttggtttatatgccctcaagtaaaagaaaagccgaaagggttaatcatatttgaaaaccatattttattgtattttgatgagatattaaattctcaaagttttattataaattctactaagttattttatgacatgaaaagttatttatgctataaattttttgaaacacaatacctacaataaactggtatgaataattgcatcatt

SEQ ID NO: 40 - protein, 165 aa, homo sapiens
MTLEEFSAGEQKTERMDKVGDALEEVLSKALSQRTITVGVYEAAKLLNVDPDNVVLCLLAADEDDDRDVALQIHFTLIQAFCCENDINILRVSNPGRLAELLLLETDAGPAASEGAEQPPDLHCVLVTNPHSSQWKDPALSQLICFCRESRYMDQWVPVINLPER

SEQ ID NO: 41 - DNA, 1363 bp, homo sapiens
agcgtcggactaccgttggtttccgcaacttcctggattatcctcgccaaggactttgcaatatatttttccgccttttctggaaggatttcgctgcttcccgaaggtcttggacgagcgctctagctctgtgggaaggttttgggctctctggctcggattttgcaatttctccctggggactgccgtggagccgcatccactgtggattataattgcaacatgacgctggaagagctcgtggcgtgcgacaacgcggcgcagaagatgcagacggtgaccgccgcggtggaggagcttttggtggccgctcagcgccaggatcgcctcacagtgggggtgtacgagtcggccaagttgatgaatgtggacccagacagcgtggtcctctgcctcttggccattgacgaggaggaggaggatgacatcgccctgcaaatccacttcacgctcatccagtccttctgctgtgacaacgacatcaacatcgtgcgggtgtcgggcatgcagcgcctggcgcagctcctgggagagccggccgagacccagggcaccaccgaggcccgagacctgcattgtctcctggtcacgaaccctcacacggacgcctggaagagccacggcttggtggaggtggccagctactgcgaagaaagccggggcaacaaccagtgggtcccctacatctctcttcaggaacgctgaggcccttcccagcagcagaatctgttgagttgctgccacaaacaaaaaatacaataaatatttgaaccccctcccccccagcacaacccccccaaaacaacccaacccacgaggaccatcgggggcagagtcgttggagactgaagaggaagaggaggaggagaaggggagtgagcggccgcccccagggcggagatccaggagctggcggccgccgatccgatggagaaggggggacccaggccagcaggagacaggacccccgaagctgaggccttgggatggagcagaagccggagtggcggggcacgctgccgccttccccatcacggagggtccagactgtccactcgggggtggagtgagactgactgcaagccccaccctccttgagactggagctggcgtctgcatacgagagacttggttgaacttggttggtccttgtctgcaccctcgacaagaccacactttgggacttgggagctggggctgaagttgctctgtacccatgaactcccagtttgcgaattatagagacaatctattttgttacttgcacttgttattcgaaccactgagagcgagatgggaagcatagatatctatatttttatttctactatgagggccttgtaataaatttctaaagcctctg

SEQ ID NO: 42 - protein, 160 aa, homo sapiens
MTLEELVACDNAAQKMQTVTAAVEELLVAAQRQDRLTVGVYESAKLMNVDPDSVVLCLLAIDEEEEDDIALQIHFTLIQSFCCDNDINIVRVSGMQRLAQLLGEPAETQGTTEARDLHCLLVTNPHTDAWKSHGLVEVASYCEESRGNNQWVPYISLQER

SEQ ID NO: 43 - DNA, 1087 bp, homo sapiens
gcactcgctggtggtgggcgcgccgtgctgagctctggctgtcagtgtgttcgcccgcgtcccctccgcgctctccgcttgtggataactagctgctggttgatcgcactatgactctggaagaagtccgcggccaggacacagttccggaaagcacagccaggatgcagggtgccgggaaagcgctgcatgagttgctgctgtcggcgcagcgtcagggctgcctcactgccggcgtctacgagtcagccaaagtcttgaacgtggaccccgacaatgtgaccttctgtgtgctggctgcgggtgaggaggacgagggcgacatcgcgctgcagatccattttacgctgatccaggctttctgctgcgagaacgacatcgacatagtgcgcgtgggcgatgtgcagcggctggcggctatcgtgggcgccggcgaggaggcgggtgcgccgggcgacctgcactgcatcctcatttcgaaccccaacgaggacgcctggaaggatcccgccttggagaagctcagcctgttttgcgaggagagccgcagcgttaacgactgggtgcccagcatcaccctccccgagtgacagcccggcggggaccttggtctgatcgacgtggtgacgccccggggcgcctagagcgcggctggctctgtggaggggccctccgagggtgcccgagtgcggcgtggagactggcaggcggggggggcgcctggagagcgaggaggcgcggcctcccgaggaggggcccggtggcggcagggccaggctggtccgagctgaggactctgcaagtgtctggagcggctgctcgcccaggaaggcctaggctaggacgttggcctcagggccaggaaggacagactggccgggcaggcgtgactcagcagcctgcgctcggcaggaaggagcggcgccctggacttggtacagttgcaggagcgtgaaggacttagccgactgcgctgctttttcaaaacggatccgggcaatgcttcgttttctaaaggatgctgctgttgaagctttgaattttacaataaactttttgaaacaaaaaaaaaaaaaaaaaaaaaaaa

SEQ ID NO: 44 - protein, 159 aa, homo sapiens
MTLEEVRGQDTVPESTARMQGAGKALHELLLSAQRQGCLTAGVYESAKVLNVDPDNVTFCVLAAGEEDEGDIALQIHFTLIQAFCCENDIDIVRVGDVQRLAAIVGAGEEAGAPGDLHCILISNPNEDAWKDPALEKLSLFCEESRSVNDWVPSITLPE

SEQ ID NO: 45 - DNA, 20 bp, Artificial Sequence
  synthetic oligonucleotide
tcgagaaccgagtgagaggc

SEQ ID NO: 46 - DNA, 21 bp, Artificial Sequence
  synthetic oligonucleotide
cacactcggaccacatccttc

SEQ ID NO: 47 - DNA, 23 bp, Artificial Sequence
  synthetic oligonucleotide
ccaacatcctgaacctcagctac

SEQ ID NO: 48 - DNA, 20 bp, Artificial Sequence
  synthetic oligonucleotide
gccttctgcgtcacaccatt

SEQ ID NO: 49 - DNA, 22 bp, Artificial Sequence
  synthetic oligonucleotide
tgtccccactgccaacgtgtca

SEQ ID NO: 50 - DNA, 24 bp, Artificial Sequence
  synthetic oligonucleotide
agcgtcaaaggtggaggagtgggt

SEQ ID NO: 51 - DNA, 20 bp, Artificial Sequence
  synthetic oligonucleotide
accacagtccatgccatcac

SEQ ID NO: 52 - DNA, 20 bp, Artificial Sequence
  synthetic oligonucleotide
tccaccaccctgttgctgta

SEQ ID NO: 53 - DNA, 20 bp, Artificial Sequence
  synthetic oligonucleotide
aaaatgtccgctgggctaag

SEQ ID NO: 54 - DNA, 21 bp, Artificial Sequence
  synthetic oligonucleotide
aggtcccagtccgagatgtag

SEQ ID NO: 55 - DNA, 18 bp, Artificial Sequence
  synthetic oligonucleotide
gccagcgccatgaggagc

SEQ ID NO: 56 - DNA, 20 bp, Artificial Sequence
  synthetic oligonucleotide
gtatccagcctgagcagtgc

SEQ ID NO: 57 - DNA, 20 bp, Artificial Sequence
  synthetic oligonucleotide
attgccattttcgctttagg

SEQ ID NO: 58 - DNA, 21 bp, Artificial Sequence
  synthetic oligonucleotide
acacgctgggaagaccgaggc

SEQ ID NO: 59 - DNA, 18 bp, Artificial Sequence
  synthetic oligonucleotide
cgacacccccgcccgcct

SEQ ID NO: 60 - DNA, 24 bp, Artificial Sequence
  synthetic oligonucleotide
acaccatgaaggcattcatgggcc

SEQ ID NO: 61 - DNA, 18 bp, Artificial Sequence
  synthetic oligonucleotide
accccgaccctgggtctt

SEQ ID NO: 62 - DNA, 18 bp, Artificial Sequence
  synthetic oligonucleotide
gccactgactccggagga

SEQ ID NO: 63 - DNA, 21 bp, Artificial Sequence
  synthetic oligonucleotide
aagggagatccggagcgaata

SEQ ID NO: 64 - DNA, 21 bp, Artificial Sequence
  synthetic oligonucleotide
ggaggctgctggttttccact

SEQ ID NO: 65 - DNA, 22 bp, Artificial Sequence
  synthetic oligonucleotide
gaaggagaagctggagcaaaac

SEQ ID NO: 66 - DNA, 20 bp, Artificial Sequence
  synthetic oligonucleotide
gagaggcaacctggagaatt

SEQ ID NO: 67 - DNA, 20 bp, Artificial Sequence
  synthetic oligonucleotide
caaaaaccctggcacaaact

SEQ ID NO: 68 - DNA, 20 bp, Artificial Sequence
  synthetic oligonucleotide
ccagaggaaaggacactggt

SEQ ID NO: 69 - DNA, 18 bp, Artificial Sequence
  synthetic oligonucleotide
tgatttgtgggcctgaag

SEQ ID NO: 70 - DNA, 18 bp, Artificial Sequence
  synthetic oligonucleotide
gatgcctggtgaacccga

SEQ ID NO: 71 - DNA, 20 bp, Artificial Sequence
  synthetic oligonucleotide
aaccagaacacgtggtttcc

SEQ ID NO: 72 - DNA, 20 bp, Artificial Sequence
  synthetic oligonucleotide
tgcaccaggtctgagtgttc

SEQ ID NO: 73 - DNA, 20 bp, Artificial Sequence
  synthetic oligonucleotide
gctcagacaccatggggaag

SEQ ID NO: 74 - DNA, 22 bp, Artificial Sequence
  synthetic oligonucleotide
ccatgagaagtatgacaacagc

SEQ ID NO: 75 - DNA, 22 bp, Artificial Sequence
  synthetic oligonucleotide
ccatgagaagtatgacaacagc

SEQ ID NO: 76 - DNA, 19 bp, Artificial Sequence
  synthetic oligonucleotide
ttctagacggcaggtcagg

SEQ ID NO: 77 - DNA, 26 bp, Artificial Sequence
  synthetic oligonucleotide
gaggagttgagagggtgattggtttt

SEQ ID NO: 78 - DNA, 27 bp, Artificial Sequence
  synthetic oligonucleotide
ggagagggggttaagtatttgggtttt

SEQ ID NO: 79 - DNA, 21 bp, Artificial Sequence
  synthetic oligonucleotide
cgaaaaaactactcaacccct

SEQ ID NO: 80 - DNA, 22 bp, Artificial Sequence
  synthetic oligonucleotide
tccactttattacccaaactaa

SEQ ID NO: 81 - DNA, 30 bp, Artificial Sequence
  synthetic oligonucleotide
ggaaaatggagttagttgaaatttttgttt

SEQ ID NO: 82 - DNA, 23 bp, Artificial Sequence
  synthetic oligonucleotide
ggaatttaaggtgtatgtatttt

SEQ ID NO: 83 - DNA, 28 bp, Artificial Sequence
  synthetic oligonucleotide
ccacccctataatcccaataaattaaaa

SEQ ID NO: 84 - DNA, 20 bp, Artificial Sequence
  synthetic oligonucleotide
aaccaacctaaccaacataa

SEQ ID NO: 85 - DNA, 33 bp, Artificial Sequence
  synthetic oligonucleotide
tgattaaataagttttagtttttttttagtttt

SEQ ID NO: 86 - DNA, 22 bp, Artificial Sequence
  synthetic oligonucleotide
ccatgagaagtatgacaacagc

SEQ ID NO: 87 - DNA, 27 bp, Artificial Sequence
  synthetic oligonucleotide
taagtatgagtagttttggttaggttt

SEQ ID NO: 88 - DNA, 30 bp, Artificial Sequence
  synthetic oligonucleotide
ttccatatccttatttcatattaatacata

SEQ ID NO: 89 - DNA, 22 bp, Artificial Sequence
  synthetic oligonucleotide
tcccacagactcctgaagaata

SEQ ID NO: 90 - DNA, 21 bp, Artificial Sequence
  synthetic oligonucleotide
ttgttccccttttaaggcttt

SEQ ID NO: 91 - DNA, 21 bp, Artificial Sequence
  synthetic oligonucleotide
gagtacagtggcgcgatatcg

SEQ ID NO: 92 - DNA, 20 bp, Artificial Sequence
  synthetic oligonucleotide
cgggagaatcccttgaacct

SEQ ID NO: 93 - DNA, 20 bp, Artificial Sequence
  synthetic oligonucleotide
gtggctcacgcctttaatca

SEQ ID NO: 94 - DNA, 20 bp, Artificial Sequence
  synthetic oligonucleotide
ccaggctggtcttgaattcc

SEQ ID NO: 95 - DNA, 18 bp, Artificial Sequence
  synthetic oligonucleotide
accccaatgccactttca

SEQ ID NO: 96 - DNA, 19 bp, Artificial Sequence
  synthetic oligonucleotide
agtcatcctcgcagatgct

SEQ ID NO: 97 - DNA, 21 bp, Artificial Sequence
  synthetic oligonucleotide
aggttaaagtgcacccaggtt

SEQ ID NO: 98 - DNA, 19 bp, Artificial Sequence
  synthetic oligonucleotide
caggcccttcttgctagct

SEQ ID NO: 99 - DNA, 20 bp, Artificial Sequence
  synthetic oligonucleotide
aacgctgagtgctgaaagga

SEQ ID NO: 100 - DNA, 20 bp, Artificial Sequence
  synthetic oligonucleotide
gtcagaccttgctgccaaag

SEQ ID NO: 101 - DNA, 19 bp, Artificial Sequence
  synthetic oligonucleotide
gggtgggtaagcaagaact

SEQ ID NO: 102 - DNA, 19 bp, Artificial Sequence
  synthetic oligonucleotide
aatgttcgtgtgccaatta

SEQ ID NO: 103 - DNA, 19 bp, Artificial Sequence
  synthetic oligonucleotide
acggcagcttgcacctcta

SEQ ID NO: 104 - DNA, 20 bp, Artificial Sequence
  synthetic oligonucleotide
ctttctagcaacccgtttgc

SEQ ID NO: 105 - RNA, 19 nt, Artificial Sequence
  synthetic ribonucleotide
cgagacgcauuucguacuu

SEQ ID NO: 106 - RNA, 19 nt, Artificial Sequence
  synthetic ribonucleotide
cgagacgcauuucguacuu

SEQ ID NO: 107 - RNA, 19 nt, Artificial Sequence
  synthetic ribonucleotide
ggacuuugauagcaacuuc

SEQ ID NO: 108 - RNA, 19 nt, Artificial Sequence
  synthetic ribonucleotide
ggacuuugauagcaacuuc

SEQ ID NO: 109 - RNA, 20 nt, Artificial Sequence
  synthetic ribonucleotide
uucaaaaauguccgcugggc

SEQ ID NO: 110 - RNA, 20 nt, Artificial Sequence
  synthetic ribonucleotide
uucaaaaauguccgcugggc

SEQ ID NO: 111 - RNA, 19 nt, Artificial Sequence
  synthetic ribonucleotide
uagcaacuuccaggaaugu

SEQ ID NO: 112 - RNA, 19 nt, Artificial Sequence
  synthetic ribonucleotide
aagcaaccuccuggaaugu

SEQ ID NO: 113 - RNA, 19 nt, Artificial Sequence
  synthetic ribonucleotide
cggcugccacguggaauug

SEQ ID NO: 114 - RNA, 19 nt, Artificial Sequence
  synthetic ribonucleotide
ugguuuacaugucgacuaa

SEQ ID NO: 115 - RNA, 19 nt, Artificial Sequence
  synthetic ribonucleotide
cgagaugcauuucguaugu

SEQ ID NO: 116 - RNA, 19 nt, Artificial Sequence
  synthetic ribonucleotide
cgagacgcauuucguacuu

SEQ ID NO: 117 - RNA, 19 nt, Artificial Sequence
  synthetic ribonucleotide
ggauuuugaaagcaaccuc

SEQ ID NO: 118 - RNA, 19 nt, Artificial Sequence
  synthetic ribonucleotide
ggacuuugauagcaacuuc

SEQ ID NO: 119 - RNA, 20 nt, Artificial Sequence
  synthetic ribonucleotide
uucaaaaauguccgcugggc

SEQ ID NO: 120 - RNA, 20 nt, Artificial Sequence
  synthetic ribonucleotide
uucaaaaauguccgcugggc

SEQ ID NO: 121 - RNA, 19 nt, Artificial Sequence
  synthetic ribonucleotide
aagcaaccuccuggaaugu

SEQ ID NO: 122 - RNA, 19 nt, Artificial Sequence
  synthetic ribonucleotide
aagcaaccuccuggaaugu

SEQ ID NO: 123 - RNA, 19 nt, Artificial Sequence
  synthetic ribonucleotide
uggcugccacguggaauug

SEQ ID NO: 124 - RNA, 19 nt, Artificial Sequence
  synthetic ribonucleotide
ugguuuacaugucgacuaa

SEQ ID NO: 125 - DNA, 385 bp, homo sapiens
ggagagggggttaagtatttgggtttttgaagaatatggaggtgtgggagtgattttagatagttgggatgtgtagagtttgagagagtgttagggagcgggttgggagttgaaagttgggtgtggtggtttacgtttttaattatgatattgggcggtagaggcgggaggattttttgaggataggaatttaagattagtttgggtaatatagtaaggttttatttttattaaaaataaaaaaattaatagggtatagtggtttaagtttgtagttttagttatttaggaggttggagtagaaggattgttttggtttagtagatcgaggttatattgagttattattgtattttattgtattttagtttgggtaataaagtga

SEQ ID NO: 126 - DNA, 249 bp, homo sapiens
ggaatttaaggtgtatgtattttttattttttttttttttttttgagacgtagtttcgttttgttgtttaggttggagtatagtggcgcgatatcggtttattataatttttgttttttaggtttaagggattttttcgttttagtttttagagtagttgggattatagatatttattattacgtgtggttaatttttgtatttttagtagagagggggtttcgttatgttggttaggttggttttaaa

(19) United States
(12) Patent Application Publication
(10) Pub. No.: US 2013/0011380 A1
(43) Pub. Date: Jan. 10, 2013

(54) Use of Cytidine Deaminase-Related Agents to Promote Demethylation and Cell Reprogramming

(76) Inventors: Helen M. Blau, Menlo Park, CA (US); Nidhi Bhutani, Stanford, CA (US); Jennifer Brady, Stanford, CA (US); Mara Damian, Campbell, CA (US)

(21) Appl. No.: 13/511,989

(22) PCT Filed: Dec. 17, 2010
(86) PCT No.: PCT/US10/60976

§ 371 Date: Sep. 18, 2012

Related U.S. Application Data
(60) Provisional application No. 61/284,519, filed on Dec. 18, 2009.

(51) Int. Cl. A61K 38/50; C12N 15/87; C12N 5/09; A61K 48/00; C12Q 1/68; G01N 27/62; G01N 33/53; C12N 5/071; C12N 5/0735

(52) U.S. Cl. 424/94.6; 435/375; 435/455; 435/377; 435/6.11; 435/6.1; 435/6.12; 435/7.1; 514/44 R

(57) ABSTRACT

Methods, compositions and kits for modulating demethylation in a mammalian cell are provided. Also provided are methods, compositions and kits for screening candidate agents for activity in modulating genomic DNA demethylation in mammalian cells. These methods, compositions and kits find use in producing induced pluripotent stem cells (iPS) and somatic cells in vitro and for treating human disorders including cancer and disorders arising from defects in genomic imprinting.

### CROSS REFERENCE TO RELATED APPLICATIONS

Pursuant to 35 U.S.C. §119 (e), this application claims priority to the filing date of the U.S. Provisional Patent Application Ser. No. 61/284,519 filed Dec. 18, 2010; the disclosure of which are herein incorporated by reference.

### GOVERNMENT RIGHTS

This invention was made with government support under AG009521 and AG024987 awarded by the National Institutes of Health. The government has certain rights in the invention.

### FIELD OF THE INVENTION

This invention pertains to methods and compositions for inducing the demethylation of genomic DNA in mammalian cells, and methods and compositions for screening candidate agents for activity in modulating genomic DNA demethylation in mammalian cells.

### BACKGROUND OF THE INVENTION

Reprogramming of somatic cell nuclei to pluripotency or to somatic cells of another cell lineage by the introduction of a few factors has enabled the generation of patient-specific induced pluripotent cells (iPS) and patient-specific somatic cells, major breakthroughs in the field of regenerative medicine. However, these processes are slow (2-3 weeks) and asynchronous, and the frequency is low (<0.1%) (see, e.g., Takahashi, K. et al. (2007) Cell 131: 861-72; Takahashi, K. & Yamanaka, S. (2006) Cell 126: 663-76; Wernig, M. et al. (2007) Nature 448: 318-24; Wernig, M. et al. (2008) Nat Biotechnol), with DNA demethylation being a bottleneck (Mikkelsen, T. S. et al. (2008) Nature 454, 49-55). The elucidation of mechanisms regulating DNA demethylation in mammalian cells and the identification of agents that will promote demethylation are therefore of clinical and research interest. The present invention addresses these issues.

Publications.

A description of mechanisms underlying DNA demethylation in zebrafish may be found at Rai, K. et al. (2008) “DNA demethylation in Zebrafish Involves the Coupling of a Deaminase, a Glycosylase, and Gadd45.” Cell 135:1201-1212.

### SUMMARY OF THE INVENTION

Methods, compositions and kits for modulating demethylation in a mammalian cell are provided. These methods, compositions and kits find use in directing reprogramming of cell fate, for example in producing induced pluripotent stem cells (iPS) from somatic cells and in redirecting somatic cells to a different cell fate. Somatic cells may be produced in vitro and in vivo, for example for use in treating human disorders which arise from or are compounded by defects in methylation, e.g. cancers and disorders associated with aberrant genomic imprinting. Also provided are methods, compositions and kits for screening candidate agents for activity in modulating genomic DNA demethylation in mammalian cells.

In one aspect of the invention, a method is provided for decreasing the amount of genomic DNA methylation in a mammalian cell, including decreasing methylation of nucleotides in promoter regions that control expression of gene(s) of interest. The method comprises contacting an initial mammalian cell with an effective amount of an agent that promotes cytidine deaminase (CD) activity, for example where the mammalian cell is a somatic cell, including somatic cells that are demethylation-permissive.

In some embodiments, the agent that promotes CD activity is an Activation-induced Cytidine Deaminase (AID) polypeptide or a nucleic acid encoding an AID polypeptide. In some embodiments, the agent that promotes CD activity is an Apolipoprotein B RNA Editing Catalytic Component (APOBEC) polypeptide or a nucleic acid encoding an APOBEC polypeptide. In some embodiments, the cell is also contacted with a polypeptide from Table 5. In certain embodiments, the polypeptide from Table 5 is an agent that promotes the conversion of methylated cytosine to hydroxylated methyl cytosine, e.g. a tet protein, e.g. tet 1 or tet2. In some embodiments, the contacting step is effected in vitro.

In some embodiments, the initial mammalian cell is a somatic cell, e.g. a demethylation-permissive somatic cell. In some such embodiments, the cell that is produced is an induced pluripotent stem cell (iPS). In some embodiments, the method further comprises the step of contacting the somatic cell with one or more factors that promote an iPS cell fate. In some embodiments, the cell that is produced is a somatic cell of a different lineage than that of the starting cell. In some such embodiments, the method further comprises the step of contacting the somatic cell with one or more factors that promote a desired somatic cell fate.

In some embodiments, the initial cell is a pluripotent stem cell, e.g. a demethylation-permissive pluripotent stem cell. In some such embodiments, the cell that is produced from the pluripotent stem cell is a somatic cell. In some such embodiments, the method further comprises the step of contacting the pluripotent stem cell with one or more factors that promote a desired somatic cell fate.

In some embodiments, the contacting step is effected in vivo, in a subject in need of genomic DNA demethylation therapy. In some such embodiments, the initial cell is a tumor cell, e.g. a demethylation-permissive tumor cell, and the subject is a subject suffering from cancer. In other such embodiments the initial cell is a non-transformed somatic cell, e.g. a demethylation-permissive somatic cell.

In one aspect of the invention, a method of screening candidate agents for activity in modulating genomic DNA demethylation activity in a cell is provided. In such methods, a first population of cells is contacted in vitro with an effective amount of an agent that promotes cytidine deaminase (CD) activity. A subpopulation of this population is then contacted with a candidate agent, while a second population, i.e. a control population, is not contacted with the candidate agent. The characteristics of the candidate agent-contacted subpopulation are then compared to the characteristics of the subpopulation of cells that were not contacted with the candidate agent, where differences in the characteristics of the cells between the first subpopulation and the second subpopulation indicates that the candidate agent modulates genomic DNA demethylation activity in a cell.

In some embodiments, the agent that promotes CD activity is an AID polypeptide or a nucleic acid that encodes an AID polypeptide. In some embodiments, the cells of the first population are tumor cells, i.e. cells from a tumor. In certain embodiments, a candidate agent that modulates genomic DNA demethylation in the tumor cells is an agent that modulates tumor growth in a cancer.

In some embodiments, the cells of the first population are somatic cells, or heterokaryons produced from ES cells and somatic cells. In some embodiments, the candidate agent that modulates the genomic DNA demethylation of the somatic cell DNA is an agent that modulates the induction of somatic cells to become iPS cells.

In one aspect of the invention, a method is provided for identifying proteins with activity in modulating the DNA demethylation activity of a cytidine deaminase. In such methods, a population of cells is contacted with a nucleic acid comprising sequence encoding the cytidine deaminase, the cytidine deaminase is precipitated from a crude protein extract of the cells, and the immunoprecipitate is subjected to mass spectroscopy, wherein the one or more proteins identified by mass spectroscopy is critical to the demethylation activity of the cytidine deaminase. In some embodiments, the cytidine deaminase is AID or APOBEC. In some embodiments, the protein that is identified is a protein in Table 5.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Isolation and characterization of mouse ES cells X human fibroblast heterokaryons. a, Heterokaryon fusion scheme. GFP⁺ mouse ES cells (mES) were co-cultured with DsRed+ human primary fibroblasts (hFb) in a 2:1 ratio and then fused using polyethylene glycol (PEG). b, FACS profiles of GFP⁺ mES, DsRed⁺ hFb, and GFP⁺ DsRed⁺ heterokaryons sorted 2 days after fusion (1st sort Het). The heterokaryon population is further resorted (2nd sort Het) to an enrichment of ˜80% of the population (Enrichment) and analyzed. Sorting gate used for heterokaryon isolation is shown. c, A representative image of GFP⁺ and DsRed⁺ heterokaryons twice sorted and cytospun 2 days post fusion is shown. Nuclei in heterokaryons remain distinct and unfused. Hoechst 33342 (blue) denotes the nuclei, and the heterokaryon shown has 3 bright mouse nuclei and 1 uniformly stained human nucleus. Scale bar=50 μm. d, GFP+DsRed+ heterokaryons were sorted twice 2 days post fusion, cytospun and stained for Ki-67 (blue) to assess cell division. The heterokaryon indicated by GFP fluorescence has two distinct nuclei (arrow) that are negative for Ki-67 (blue) in contrast to the mononuclear cells (arrowheads) that stain positive for Ki-67. Scale bar=50 μm. e, Heterokaryons sorted and cytospun on days 1, 2 and 3 post fusion were scored based on Ki-67 staining, and 98(±2) % heterokaryons were non-dividing (mean±s.e.m., p<0.05). f, Heterokaryons, generated using GFP− ES cells, (see supplementary methods) were enriched using a human fibroblast marker Thy1.1 on day 1 post fusion, cytospun and stained for BrdU (green) and nuclei (blue) using Hoechst 33258. The indicated heterokaryon (arrow) has 3 uniformly stained human nuclei and 1 bright, punctate mouse nucleus, and is negative for BrdU. In contrast, the indicated human mononuclear cell (arrowhead) stains positive for BrdU. Scale bar=50 μm. g, Heterokaryons enriched and cytospun on days 1, 2 and 3 post fusion were scored based on nuclear and BrdU staining. DNA replication did not occur in 94(±3) % heterokaryons (mean±s.e.m., p<0.05).

FIG. 2. Time course of human fibroblast pluripotency gene expression in heterokaryons at the single cell level. a, Human specific primers against Oct4, Nanog and GAPDH were used for RT-PCR of unfused co-cultures on day 0 and heterokaryons (mES×hFb) isolated on days 1, 2 and 3 post fusion. Both hOct4 and hNanog are upregulated in heterokaryon samples showing a rapid initiation of reprogramming of the human fibroblast nuclei in heterokaryons. b, Real time PCR was used to assess the upregulation of Oct4 (gray) and Nanog (black) in heterokaryons isolated on days 1, 2 and 3 post fusion using human specific primers (mean±s.d.). Unfused co-cultures served as day 0 controls and the expression of hOct4 and hNanog was normalized to hGAPDH expression. Statistically significant differences were observed between the gene expression of hOct4 and hNanog on day 0 and on days 1, 2 and 3 (* denotes p<0.03). Data shown are from three independent fusion experiments. c, Single heterokaryon nested PCR to assess the efficiency of reprogramming in the heterokaryon population. Heterokaryons were enriched to 80% and isolated as single cells on day 3 post fusion. Direct reverse transcription and nested PCR were performed simultaneously using human-specific primers for GAPDH (G), Oct4 (O) and Nanog (N) on single heterokaryons as indicated. 12 heterokaryons analyzed from a single fusion experiment are shown. 10 and 31 heterokaryons analyzed from 2 additional fusion experiments are shown in FIG. 7. d, The fraction of heterokaryons expressing Oct4 only as well as both Oct4 and Nanog is 70±13%, showing that a high proportion of heterokaryons initiate reprogramming towards pluripotency. Data shown are a summary of 3 independent fusion experiments (mean±s.d.).

FIG. 3. Time course of DNA demethylation at human fibroblast pluripotency gene promoters in heterokaryons. a, Bisulfite sequencing analysis of methylation status of the human Oct4 and Nanog promoter in heterokaryons. Both human Oct4 and Nanog promoters in heterokaryons show rapid and progressive DNA demethylation on days 1, 2 and 3 post fusion compared to the co-culture control. White circles indicate unmethylated and black circles indicate methylated CpG dinucleotides. b, Percent demethylation at the human Oct4 promoter in heterokaryons post fusion showing a progressive increase in demethylation to 80% on day 3. c, Percent demethylation at the human Nanog promoter in heterokaryons post fusion showing a progressive increase in demethylation to 56% on day 3. At least 10 clones were analyzed at each time-point in 2 to 3 independent experiments; 10 representative clones are shown.

FIG. 4. Requirement of AID-dependent DNA demethylation for initiation of human fibroblast reprogramming towards pluripotency in heterokaryons. a, AID and human pluripotency gene expression in heterokaryons subjected to siRNA treatment, as assessed by real time PCR. si-1, 2, 3 and 4 are distinct siRNAs directed toward AID. Heterokaryons isolated on Day 2 post fusion were treated with si-3 and si-4, and heterokaryons isolated on Day 3 were treated with si-1 and si-2. Total levels of mouse and human AID transcripts was assessed using a set of degenerate primers while human-specific primers were used for hOct4 and hNanog. Gene expression was normalized internally to GAPDH (degenerate primers) for AID expression and to hGAPDH for human Oct4 and Nanog expression. The samples were then normalized to the corresponding Day 2 or Day 3 sample treated with the control siRNA, and a representative siControl (100%) is displayed. AID expression in heterokaryons treated with si-3, 4, 1, and 2 were reduced compared to the control. Knockdown of AID by all the 4 siRNAs blocked the expression of the pluripotency genes, hOct4 and hNanog. b, Human Oct4 and Nanog promoters on days 2 and 3 post fusion upon AID knockdown by si-3/si-4 and si-1/si-2, respectively, remain methylated showing an inhibition of demethylation and supporting the role of AID in DNA demethylation and nuclear reprogramming in heterokaryons. c, Percent demethylation at the human Oct4 and Nanog promoters upon AID knockdown with si-3/si-4, and si-1/si-2 showed a block in demethylation compared to their respective Day 2 and Day 3 control samples treated with siControl. d, Human AID is recruited to the promoter of human Nanog and Oct4 genes in fibroblasts, in which the promoters are heavily methylated. Chromatin immunoprecipation with anti-AID antibody was performed in mES and hFb. AID occupancy is shown relative to background IgG signal (mean±SE). Significant AID binding was detected in hFb as well as mES for positive controls, Cμ and Cdx2, respectively (p<0.02). AID binding to the methylated promoters of hOct4 and hNanog was significant in human fibroblasts (p<0.02) while no significant binding was observed for the unmethylated Oct4 and Nanog promoters in mouse ES cells. e, Model of AID-dependent DNA demethylation in reprogramming toward pluripotency in heterokaryons. The other putative components of a mammalian DNA demethylase complex (X, Y, and Z) that may act together with the deaminase AID remain to be identified.

FIG. 5. Schemes for heterokaryon generation, siRNA knockdown, hAID overexpression, and rescue experiments.

FIG. 6. Thy1.1 enrichment of heterokaryons for BrdU experiments. a, Specificity of Thy1.1 for human fibroblasts. GFP+ mouse ES cells do not bind the human-specific Thy1.1 antibody as shown in a (Top panel), while 100% of dsRed+ human fibroblasts bind Thy1.1 (bottom panel) showing that Thy1.1 can be specifically used as a cell-surface marker for human fibroblasts. b, Heterokaryon enrichment using Thy1.1 antibody. Biotinylated Thy1.1 was used to label PEG-treated mES and hFb co cultures, and streptavidin magnetic beads were used to enrich for human fibroblasts and heterokaryons (mES X hFb). 0.1-1% heterokaryons are present in the PEG-treated mES and Fb co-cultures before enrichment as shown in b (bottom left) while after magnetic bead enrichment, the Thy1.1 positive heterokaryons and fibroblasts were 10% and 80%, respectively.

FIG. 7. Heterokaryons do not undergo DNA replication. Heterokaryons, generated using GFP⁻ (non GFP) ES cells and human fibroblasts, were enriched using a human fibroblast marker, Thy1.1, on day 1 post fusion (see supplementary methods), cytospun and stained for BrdU (green) and nuclei (blue) using Hoechst 33258. The indicated heterokaryon (arrow) has 3 uniformly stained human nuclei and 1 bright, punctate mouse nucleus, and is negative for BrdU, showing that there is no DNA replication in heterokaryons. In contrast, the indicated mononuclear cells (arrowheads) stain positive for BrdU. Magnetic streptavidin beads are visible on the surface of the cells in the immunofluorescence images as small black circles. Scale bar=50 microns.

FIG. 8. Validation of human-specific primers to study pluripotency gene activation in interspecies heterokaryons. a, Human-specific primers designed for Oct4 and Nanog selectively amplify human transcripts from human ES cells (hES) while not detecting the Oct4 and Nanog transcripts from mouse ES cells (mES). b, Human and bisulfite-specific primers designed to assess the methylation status of the human Oct4 and Nanog promoters only amplified a product from hFb and not from mES. c, Species specificity of nested PCR primers designed to assess expression of human GAPDH, Oct4 and Nanog transcripts in single heterokaryons. A product is detected in both hES cells and human primary fibroblasts (hFb) using the human GAPDH primers, while no product is detected in mES. Human Oct4 and Nanog transcripts are detected only in hES cells, and no product is detected in hFb or mES.

FIG. 9. Human pluripotency gene expression in co-culture controls over time. Day 0 and day 3 unfused co-cultures of mouse ES cells and hFb were analyzed by real time PCR using human-specific primers against the pluripotency genes Oct4 and Nanog. Gene expression was normalized to hGAPDH, and to the day 0 control to obtain the fold change in gene expression. The data show that expression of hOct4 and hNanog remain unchanged from day 0 through day 3.

FIG. 10. Activation and expression of human pluripotency genes in heterokaryons. Human-specific primers against the pluripotency genes Essrb, TDGF1, Sox2 and the cell cycle regulators Klf4 and c-myc were used for real time PCR of unfused co-cultures on day 0 and of heterokaryons (mES X hFb) isolated on day 2 post fusion. Gene expression was normalized to hGAPDH, and to the day 0 control to obtain the fold change in gene expression. Essrb, TDGF1 and c-myc are induced in heterokaryon samples. Sox 2, which is already expressed in human fibroblasts, does not increase further nor does Klf4, which is known to be interchangeable with Essrb (Feng, B. et al. (2009) Nat Cell Biol 11, 197-203).

FIG. 11. Efficiency of reprogramming in single heterokaryons. In FIG. 2c, results from 12 heterokaryons are shown derived from a single fusion experiment. Here, pluripotency gene expression in heterokaryons derived from two additional independent fusion experiments are shown (n=10 and 31, respectively.) Summary of the results of all the 3 independent experiments and statistics are shown in FIG. 2d.

FIG. 12. Relative AID mRNA expression. AID transcript levels were assessed by real time PCR in a B-lymphocyte cell line (Ramos), mouse embryonic stem cells (mES) and human fibroblasts (hFb). Transcript levels are normalized to GAPDH. The ratio of AID expression in Ramos, mES, and hFb is approximately 100:15:5.

FIG. 13. siRNAs directed to AID target sequences: alignment for human and mouse. The siRNA target sequences and their corresponding target in the human (SEQ ID NO:105—SEQ ID NO:114) and mouse (SEQ ID NO:115-SEQ ID NO:124) AID mRNA are shown, as well as their relative position along the AID transcript. Mismatches (*) are indicated above the target sequence.

FIG. 14. Efficacy of AID knockdown in mouse ES cells and human fibroblasts. Knockdown of AID was assessed by real time PCR in mouse ES cells and human fibroblasts on day 3 post-siRNA transfection. AID si-1, 2, 3 and 4 are distinct siRNAs directed toward AID (sequences shown in FIG. 13) and reduced AID transcripts in mouse ES cells (normalized to GAPDH) by 81 (±13) %, 79(±12) %, 70(±8) %, and 99(±0.1) %, respectively, at day 3 posttransfection as compared to the control siRNA. In human fibroblasts, AID mRNA levels were reduced by 46(±11)°/0, 72(±23) %, 99(±0.1) % and 99(±0.1) % by siRNA 1, 2, 3 and 4, respectively.

FIG. 15. Detection of AID protein and knockdown in mouse ES cells. a, Detection of AID protein after immunoprecipitation from 2 mg of mouse ES whole cell lysate. 1% of input (20 μg) was loaded. b, Detection of AID protein levels in concentrated mouse ES cell lysates (170 μg) 3 days post-transfection with si-1. α-tubulin is shown as a loading control. c, Quantification of AID protein levels, normalized to α-tubulin. The AID protein levels in ES cells treated with si-1 were reduced to 12% compared to the siControl sample.

FIG. 16. Compiled bisulfite sequence data for human Oct4 and Nanog promoters in heterokaryons. A total of 330 clones were sequenced.

FIG. 17. Over-expression of human AID does not accelerate the onset of reprogramming in heterokaryons. Bisulfite sequencing analysis of methylation status of the human Oct4 and Nanog promoters in fibroblasts in heterokaryons on day 1 post fusion, with or without transient over-expression of human AID (hAID) (FIG. 5). a, hAID levels were assessed by real time PCR and found to be upregulated 2 and 4 fold respectively in two separate fusion experiments, in the day 1 heterokaryons. b,d, The extent of DNA demethylation of the human Oct4 and Nanog promoters does not increase upon hAID over-expression. Similar results were obtained for two independent fusion experiments. White circles indicate unmethylated and black circles indicate methylated CpG dinucleotides. At least 10 clones were analyzed in two independent fusion experiments; 10 representative clones are shown. c,e, Percent demethylation observed at the human Oct4 and Nanog promoters in heterokaryons on day 1 post fusion, with or without transient over-expression of hAID. DNA demethylation at the Oct4 and Nanog promoters does not increase when hAID is over-expressed.

FIG. 18. Over-expression of human AID rescues the initiation of reprogramming during transient knockdown of AID in heterokaryons. Rescue experiments were performed by over-expressing human AID (hAID) in heterokaryons transfected with an si-RNA targeting AID (si-1) (see FIG. 5). a, hAID levels were assessed by real time PCR and found to be upregulated 2.5 and 4 fold respectively in two separate fusion experiments, in day 2 heterokaryons. b, Over-expression of hAID partially rescues the expression of the pluripotency gene hOct4 and completely rescues hNanog gene expression in day 2 heterokaryons relative to the control, as assessed by real time PCR using human-specific primers. Expression levels are normalized to hGAPDH in the same day 2 sample and then to the day 0 control. c,d Heterokaryons isolated on day 2 post fusion were subjected to bisulfite sequencing analysis for the methylation status of the human Oct4 and Nanog promoters. Both promoters show demethylation, indicating that the block in reprogramming caused by AID downregulation is overcome by hAID over-expression, with a full rescue of Nanog promoter demethylation and a partial rescue of Oct4 promoter demethylation. Oct4 promoter demethylation is rescued from 8% demethylation (si-1) to 22% demethylation (hAID+si-1) as compared to the control levels of 72%. Nanog promoter demethylation is rescued from 31% demethylation (si-1) to 51% demethylation (hAID+si-1). Complete rescue is observed, as compared to the control levels of 47%.

FIG. 19. Map of the human Oct4 (SEQ ID NO:125) and Nanog (SEQ ID NO:126) promoters showing CpG density surveyed in the bisulfite specific sequencing and ChIP assays. Sequences given are for bisulfite specific amplicons. CpG sites in the human Oct4 and Nanog promoters are shown in boldface. Regions of ChIP primer coverage (real time PCR amplicons) are indicated. Distance from ATG start codon is shown.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Methods, compositions and kits for modulating demethylation in a mammalian cell are provided. Also provided are methods, compositions and kits for screening candidate agents for activity in modulating the level of genomic DNA demethylation activity in mammalian cells. These methods, compositions and kits find use in producing induced pluripotent stem cells (iPS) and somatic cell in vitro and for treating human disorders including cancer and disorders arising from defects in genomic imprinting in vivo. These and other objects, advantages, and features of the invention will become apparent to those persons skilled in the art upon reading the details of the compositions and methods as more fully described below.

By “DNA methylation” or simply “methylation” it is meant the addition of a methyl group to DNA. Reactions in which methyl groups are added to DNA are catalyzed by the enzyme DNA methyltransferase (DNMT). In vertebrates, DNA methylation typically occurs on the nucleotide cytosine, usually at CpG sites (cytosine-phosphate-guanine sites; that is, where the cytosine is directly followed by a guanine in the DNA sequence). This results in the conversion of the cytosine to 5-methylcytosine, referred to interchangeably herein as “5-methylcytosine”, “5-meC”, and “methylated cytosine”. The added methyl group alters the structure of the cytosine without altering its base-pairing properties. The extent of methylation of CpG sequences and islands, which are “GC rich” regions (i.e. made up of about 65% CG residues) is often associated with the transcriptional activity of the gene, where promoters containing highly methylated CpG islands are typically silent, and promoters containing unmethylated or less-methylated CpG islands are typically active.

By “DNA demethylation” or simply “demethylation” it is meant the conversion of CpG sequences from methylated CpG sequence to non-methylated CpG sequence.

By a “DNA demethylation-permissive cell” or “demethylation-permissive cell” it is meant a cell that is capable of having its CpG sequences converted from methylated CpG sequence to non-methylated CpG sequence. One can determine if a cell is permissive to demethylation by overexpressing a cDNA encoding Activation-induced Cytidine Deaminase (AID) (GenBank Accession No. NM_{—}020661) in the cell, providing the cell with a DNA vector comprising 5-meCpG-rich nucleotide sequence, and harvesting and analyzing the vector-supplied nucleotide sequence by, for example, bisulphite sequencing or methylase-sensitive restriction endonuclease digestion to determine if the CpG sequences of that nucleotide sequence have been demethylated.

By “cytidine deaminase activity” or “CD activity” it is meant the activity of an enzymatic pathway that results in the removal of amine groups from cytosine or 5-methylcytosine nucleosides that are attached to a ribose ring (a cytidine) or a deoxyribose ring (a deoxycytidine). Removal of an amine group from a cytosine results in a conversion of the nucleoside to a uracil, whereas removal of an amine group from a 5-methylcytosine results in a conversation of the nucleoside to a thymine. See, for example, the diagram below:

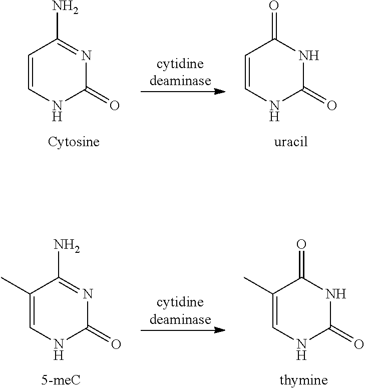

By “pluripotent stem cell” or “pluripotent cell” it is meant a cell that has the ability to differentiate into all types of cells in an organism. Pluripotent cells are capable of forming teratomas and of contributing to ectoderm, mesoderm, or endoderm tissues in a living organism. Examples of pluripotent stem cells are embryonic stem (ES) cells, embryonic germ stem (EG) cells, and induced pluripotent stem (iPS) cells.

By “embryonic stem cell” or “ES cell” it is meant a cell that a) can self-renew, b) can differentiate to produce all types of cells in an organism, and c) is derived from the inner cell mass of the blastula of a developing organism. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. No. 7,029,913, U.S. Pat. No. 5,843,780, and U.S. Pat. No. 6,200,806, the disclosures of which are incorporated herein by reference.

By “embryonic germ stem cell”, embryonic germ cell” or “EG cell” it is meant a cell that a) can self-renew, b) can differentiate to produce all types of cells in an organism, and c) is derived from germ cells and germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPS cell” it is meant a cell that a) can self-renew, b) can differentiate to produce all types of cells in an organism, and c) is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPS cells may be found in, for example, Application Nos. US20090047263, US20090068742, US20090191159, US20090227032, US20090246875, and US20090304646, the disclosures of which are incorporated herein by reference.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “reprogramming factors” it is meant one or more, i.e. a cocktail, of biologically active factors that act on a cell to alter transcription, thereby reprogramming a cell to pluripotency. In methods of the invention where reprogramming factors are provided to cells, i.e. the cells are contacted with reprogramming factors, these reprogramming factors may be provided to the cells individually or as a single composition, that is, as a premixed composition, of reprogramming factors. The factors may be provided at the same molar ratio or at different molar ratios. The factors may be provided once or multiple times in the course of culturing the cells of the subject invention.

By “efficiency of reprogramming” it is meant the ability of an in vitro culture of cells to be reprogrammed to give rise to cells of another cell type. Cells which demonstrate an enhanced efficiency of reprogramming in the presence of an agent, e.g. an agent that promotes cytidine deaminase activity, will demonstrate an enhanced ability to give rise to cells of another cell type when contacted with that agent relative to cells that were not contacted with that agent. By enhanced, it is meant that the cell cultures have the ability to give rise to the new type of cell that is at least 50%, about 100%, about 200%, about 300%, about 400%, about 600%, about 1000%, about 2000%, at least about 5000% of the ability of the cell culture that was not contacted with the agent. In other words, the cell culture produces about 1.5-fold, about 2-fold, about 3-fold, about 4-fold, about 6-fold, about 10-fold, about 20-fold, about 30-fold, about 50-fold, about 100-fold, about 200-fold more cells of the new cell type than that are produced by a population of cells that are not contacted with the agent. In some embodiments of the application, an agent that enhances the efficiency of reprogramming is an agent that decreases the amount of DNA methylation at promoters that are known in the art to become active during the acquisition of the desired cell fate, e.g. by 1.5 fold or more, i.e. by about 1.5-fold, about 2-fold, about 3-fold, about 4-fold, about 6-fold, or about 10-fold or more, relative to the amount of DNA methylation that would be observed absent the agent. In some embodiments of the application, an agent that enhances the efficiency of reprogramming is an agent that increases the amount of transcription of genes regulated by promoters that are known in the art to become active during the acquisition of the desired cell fate, e.g. by about 1.5 fold or more, i.e. by about 1.5-fold, about 2-fold, about 3-fold, about 4-fold, about 6-fold, or about 10-fold or more, relative to the amount of transcription that would be observed absent the agent.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention. Transcriptional activity from a promoter sequence may be modulated by the extent to which the promoter is methylated.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired, particularly humans.

Agents that Promote Cytidine Deaminase (CD) Activity

Methods, compositions and kits for modulating the amount of methylation in a mammalian cell are provided. In one aspect of the invention, the amount of genomic DNA methylation in a mammalian cell is decreased by contacting a cell with one or more agents that promote cytidine deaminase activity. As discussed above, cytidine deaminase (CD) activity is an enzymatic activity in which amino groups are removed from cytosines or 5-methyl cytosines in DNA or RNA. Examples of agents that promote cytidine deaminase activity that find use in the present application are polypeptides and fragments of the AID/APOBEC class of cytidine deaminases and nucleic acids that encode these polypeptides and fragments.

Activation-induced Cytidine Deaminase, also referred to as AID, AICDA, ARP2, CDA2, or HIGM2, is a cytidine deaminase that is most known for its role in the adaptive humoral immune system, deaminating cytosine residues in the DNA of the immunoglobulin locus to potentiate antibody gene diversification (somatic hypermutation and gene conversion of the immunoglobulin V gene and switch recombination of the IgC gene). The terms “AID gene product”, “AID polypeptide”, “AID peptide”, and “AID protein” are used interchangeably herein to refer to native sequence AID polypeptides, AID polypeptide variants, AID polypeptide fragments and chimeric AID polypeptides. The native sequence for AID polypeptide and the nucleic acid that encodes it may be found at GenBank Accession No. NM_{—}020661 (SEQ ID NO:1, SEQ ID NO:2).

Apolipoprotein B RNA Editing Catalytic Component proteins, also referred to as APOBEC proteins, are a family of proteins that deaminate cytidines. The terms “APOBEC gene product”, “APOBEC polypeptide”, “APOBEC peptide”, and “APOBEC protein” are used interchangeably herein to refer to native sequence APOBEC polypeptides, APOBEC polypeptide variants, APOBEC polypeptide fragments and chimeric APOBEC polypeptides. The founder member of the APOBEC family, APOBEC1, is the catalytic component of a complex that edits apolipoprotein B RNA by deaminating the cytosine 6666 to a uracil, thereby creating a premature stop codon and potentiating the tissue-specific production of a truncated apolipoprotein B polypeptide chain. Native human sequence for APOBEC1 polypeptide and the nucleic acid that encodes it may be found at GenBank Accession No. NM_{—}001644 (SEQ ID NO:3, SEQ ID NO:4). Members of the APOBEC3 family (APOBEC3F, APOBEC3G and APOBEC3H) play roles in an innate immune pathway of restriction of retroviral infection, by deaminating the cytosines in retroviral first-strand cDNA replication intermediates or generating lethal hypermutations in viral genomes; the native human sequence for APOBEC3F (also known as KA6, ARP8, MGC74891, and BK150C2.4.mRNA) may be found at GenBank Accession Nos. NM_{—}145298.5 (isoform a) (SEQ ID NO:5, SEQ ID NO:6) and NM_{—}001006666.1 (isoform b) (SEQ ID NO:7, SEQ ID NO:8); the native human sequence for APOBEC3G (also known as ARP9, CEM15, MDS019, FLJ12740, bK150C2.7 and dJ494G10.1) may be found at GenBank Accession Nos. NM_{—}021822.2 (SEQ ID NO:9, SEQ ID NO:10); and the native human sequence for APOBEC3H (also known as ARP10), may be found at GenBank Accession Nos. NM_{—}001166003.1 (isoform 1) (SEQ ID NO:11, SEQ ID NO:12), NM_{—}181773.3 (isoform 2) (SEQ ID NO:13, SEQ ID NO:14), NM_{—}001166002.1 (isoform 3) (SEQ ID NO:15, SEQ ID NO:16), and NM_{—}001166004.1 (isoform 4) (SEQ ID NO:17, SEQ ID NO:18). Other members of the APOBEC family of cytidine deaminases include APOBEC2 (also known as ARP1 and ARCD1), the native human sequence for which may be found at GenBank Accession No. NM_{—}006789 (SEQ ID NO:19, SEQ ID NO:20); APOBEC3A (also known as Phorbolin 1, ARP3, PHRBN, and bK150C2.1), the native human of sequence for which may be found at GenBank Accession No. NM_{—}145699.3 (SEQ ID NO:21, SEQ ID NO:22); APOBEC3B (also known as ARP4, ARCD3, PHRBNL, APOBEC1L, FLJ21201, bK15002.2 and DJ742C19.2), the native human sequence for which may be found at GenBank Accession No. NM_{—}004900 (SEQ ID NO:23, SEQ ID NO:24); APOBEC3C (also known as PBI, ARP5, ARDC2, ARDC4, APOBEC1L, MGC19485, and bK150C2.3), the native human sequence for which may be found at GenBank Accession No. NM_{—}014508.2 (SEQ ID NO:25, SEQ ID NO:26); and APOBEC3D (also known as ARP6, APOBEC3E, and APOBEC3DE), the native human sequence for which may be found at GenBank Accession No. NM_{—}152426.3 (SEQ ID NO:27, SEQ ID NO:28).

More information on the AID/APOBEC class of cytidine deaminases and the domains that are conserved amongst this class of proteins may be found in Conticello, S. G. et al. (2005) Molecular Biology and Evolution 22(2) 367-377, the disclosure of which is incorporated herein by reference.

In some embodiments, the agent that promotes CD activity and hence, genomic DNA demethylation is an AID polypeptide. An AID polypeptide is a polypeptide comprising AID sequence that promotes cytidine deamination. An AID polypeptide may comprise a polypeptide having a sequence identity of 50%, 55%, 60%, 65%, 70%, 75%, 80%, 85%, 90%, 95%, 98% or 100% to the full polypeptide sequence of AID or fragments of AID with cytidine deaminase activity, for example, the full-length polypeptide minus the C-terminal 10 amino acids (Barreto et al. (2003) Mol. Cell. 12(2):501-8). Such fragments are readily identifiable to one of ordinary skill in the art using common biochemical and genetic techniques that are well known in the art. Also encompassed by the subject invention are nucleic acids encoding polypeptides having a sequence identity of 50%, 55%, 60%, 65%, 70%, 75%, 80%, 85%, 90%, 95%, 98% or 100% to the polypeptide sequence of full length AID or its cytidine deaminase active domain, and vectors comprising these nucleic acids.

In some embodiments, the agent that promotes CD activity and hence, genomic DNA demethylation is an APOBEC polypeptide. An APOBEC polypeptide is a polypeptide comprising APOBEC sequence that promotes cytidine deamination. An APOBEC polypeptide may comprise a polypeptide having a sequence identity of 50%, 55%, 60%, 65%, 70%, 75%, 80%, 85%, 90%, 95%, 98% or 100% to the full polypeptide sequence of APOBEC or fragments of APOBEC with cytidine deaminase activity. Such fragments are readily identifiable to one of ordinary skill in the art using common biochemical and genetic techniques that are well known in the art. Also encompassed by the subject invention are nucleic acids encoding polypeptides having a sequence identity of 50%, 55%, 60%, 65%, 70%, 75%, 80%, 85%, 90%, 95%, 98% or 100% to the polypeptide sequence of any of the full length APOBEC polypeptides or their cytidine deaminase active domain, and vectors comprising these nucleic acids.

As mentioned above, suitable agents for use in the present invention include polypeptides and fragments of the AID/APOBEC class of cytidine deaminase proteins as well as nucleic acids that encode these polypeptides and fragments. In some embodiments, the one or more agent(s) that promote CD activity are nuclear acting, non-integrating polypeptides. In other words, the subject cells are contacted with polypeptides that promote CD activity (“CD activity polypeptides”) and act in the nucleus. By non-integrating, it is meant that the polypeptides do not integrate into the genome of the subject cell, that is, the cell in which it is desirous to promote demethylation activity.

To promote transport of CD activity polypeptides across the cell membrane, CD activity polypeptide sequences may be fused to a polypeptide permeant domain. A number of permeant domains are known in the art and may be used in the nuclear acting, non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

The CD activity polypeptides may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Other methods of preparing cytidine deaminase activity polypeptides in a cell-free system include, for example, those methods taught in U.S. Application Ser. No. 61/271,000, which is incorporated herein by reference.

The CD activity polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein. CD activity polypeptides may be produced recombinantly not only directly, but also as a fusion polypeptide with a heterologous polypeptide, e.g. a polypeptide having a specific cleavage site at the N-terminus of the mature protein or polypeptide. Expression vectors usually contain a selection gene, also termed a selectable marker. This gene encodes a protein necessary for the survival or growth of transformed host cells grown in a selective culture medium.

Following purification by commonly known methods in the art, CD activity polypeptides are provided to the subject cells by standard protein transduction methods. In some cases, the protein transduction method includes contacting cells with a composition containing a carrier agent and at least one purified CD activity polypeptide. Examples of suitable carrier agents and methods for their use include, but are not limited to, commercially available reagents such as Chariot™ (Active Motif, Inc., Carlsbad, Calif.) described in U.S. Pat. No. 6,841,535; Bioport™ (Gene Therapy Systems, Inc., San Diego, Calif.), GenomeONE (Cosmo Bio Co., Ltd., Tokyo, Japan), and ProteoJuice™ (Novagen, Madison, Wis.), or nanoparticle protein transduction reagents as described in, e.g., U.S. patent application Ser. No. 10/138,593.

In other embodiments, the one or more agents that promote CD activity are nucleic acids encoding CD activity polypeptides. Vectors used for providing nucleic acids encoding CD activity polypeptides to the subject cells will typically comprise suitable promoters for driving the expression, that is, transcriptional activation, of the nucleic acids. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10-fold, by at least about 100-fold, more usually by at least about 1000-fold. In addition, vectors used for providing the nucleic acids may include genes that must later be removed, e.g. using a recombinase system such as Cre/Lox, or the cells that express them destroyed, e.g. by including genes that allow selective toxicity such as herpesvirus TK, bcl-xs, etc

Nucleic acids encoding CD activity polypeptides may be provided directly to the subject cells. In other words, the cells are contacted with vectors comprising nucleic acids encoding the CD activity polypeptides such that the vectors are taken up by the cells. Methods for contacting cells with nucleic acid vectors, such as electroporation, calcium chloride transfection, and lipofection, are well known in the art. Vectors that deliver nucleic acids in this manner are usually maintained episomally, e.g. as plasmids or minicircle DNAs.

Alternatively, the nucleic acid may be provided to the subject cells via a virus. In other words, the cells are contacted with viral particles comprising the nucleic acid encoding the CD activity polypeptides. Retroviruses, for example, lentiviruses, are particularly suitable to such methods. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject cells are targeted by the packaged viral particles. Methods of introducing the retroviral vectors comprising nucleic acids encoding polypeptides that promote cytidine deaminase activity into packaging cell lines and of collecting the viral particles that are generated by the packaging lines are well known in the art.

In methods of the invention, the amount of genomic DNA methylation in a mammalian cell is decreased by contacting a cell with an effective amount of one or more agents that promote CD activity. The amount of an agent that is sufficient to decrease genomic DNA methylation in a cell is the amount of agent sufficient to promote CD activity in a cell, i.e. the amount sufficient to promote the removal of amino groups from cytosines and 5-methylcytosines in a cell. This amount can be empirically determined by a number of assays known in the art that measure the conversion of the cytosine or 5-methylcytosine nucleosides to uracil or thymine nucleoside, respectively, where an effective amount of an agent to decrease the amount of genomic DNA methylation in a cell is an amount that will induce the conversion of 5% or more cytosines or 5-methylcytosines, i.e. 5%, 10%, 20%, 40%, 60%, 80%, or 100%, to uracil or thymine. For example, the extent of dC deamination to dU (deoxyuracil) may be assayed by using uracil DNA glycosylase (UDG) and apurinic endonuclease (APE) as described in Bransteitter, R. et al. ((2003) PNAS 100(7):4102-4107), the disclosure of which is incorporated herein by reference. In such an assay, a DNA or RNA substrate (e.g. 100 nM) that has been 5′-end-labeled with ³²P is incubated with the agent that promotes cytidine deaminase activity. A complementary DNA strand is then annealed to the substrate followed by incubation with UDG and APE. After incubation, the reaction is terminated and the reaction products are resolved by denaturing polyacrylamide gel electrophoresis (PAGE) and visualized by phosphorimaging, where the presence of a short radioactive product corresponding to the length from labeled terminus to a cytidine is indicative of a nick at an original cytidine, reflective of CD activity at that cytidine. As another example, dC deamination may be detected by using primer elongation-dideoxynucleotide termination, also described in Bransteitter, R. et al, supra. In such an assay, a DNA or RNA substrate that is reacted with the agent that promotes CD activity is annealed to a 3-fold excess 18-mer ³²P-labeled primer, the primer is elongated by using T7 sequenase in the presence of three dNTPs plus either 2′,3′-dideoxyadenosine (ddA) or 2′,3′-dideoxyguanosine (ddG) triphosphate. The substrate-extended primer complexes are heat-denatured, and the separated strands are annealed to a complementary DNA strand and incubated with UDG and APE as described above. The products of reactions are resolved by denaturing PAGE and visualized by phosphorimaging, where deamination efficiencies are calculated from extension reactions with the ddA mix as a ratio of the band intensity opposite the C/U template compared with the integrated band intensities at and past the C template. The efficiencies may also be calculated from extension reactions with the ddG mix as a ratio of integrated band intensities past the template C to the integrated band intensities at and past the C template. In this manner, agents that promote CD activity may be identified and the effective amount of an agent that promotes CD activity may be empirically determined.

The effective amount of an agent that is sufficient to decrease the amount of genomic DNA methylation may also be determined by assaying the extent of DNA methylation following treatment with that agent. An effective amount of an agent to decrease the amount of genomic DNA demethylation in a cell is an amount that will induce a 1.5-fold or greater reduction, i.e. a 1.5-fold, a 2-fold, a 3-fold, a 4-fold, a 5-fold, a 10-fold, or a 20-fold or more reduction in the number of methylated CpG sequences in a DNA sequence. Several methods are well-known in the art for assaying the state of methylation of CpG sequences, for example, restriction endonuclease digestion and bisulphite sequencing. In restriction endonuclease digestion, CpG sequences containing 5-methylcytosine (e.g. C^{me}CGG) can be distinguished from CpG sequences containing unmethylated cytosines (CCGG) by the resistance of the 5-methylcytosine-containing sequence to cleavage with the restriction enzyme HpaII. In contrast, methylated and unmethylated CpG sequences are digested equally well by the restriction enzyme MspI. Based upon this, genomic DNA may be subjected to restriction endonuclease digestion with MspI and HpaII in separate reactions to determine a) the location of CpG sequences and b) whether these sequences are unmethylated (i.e. sensitive to HpaII restriction) or methylated (i.e. resistant to HpaII restriction). In bisulphite sequencing, treatment of DNA with bisulfite converts cytosine residues to uracil, but leaves 5-methylcytosine residues unaffected; thus, bisulfite treatment introduces specific changes in the DNA sequence that depend on the methylation status of individual cytosine residues, yielding single-nucleotide resolution information about the methylation status of a segment of DNA. Examples of regions of genomic DNA that may be assayed for their state of methylation include the promoter regions of OCT4, NANOG, RB1, CDKN2A^{INK4A}, CDKN2A^{ARF}, CDH1, CDH13, TIMP3, VHL, MLH1, MGMT, BRCA1, GSTP1, SMARCA3, RASSF1A, SOCS1, ESR1, DAPK1. Other regions of genomic DNA that may be assayed are described in Costello, J. F., et al. (2000) Nature Genet. 25:132-138, Song, F. et al., (2005) PNAS 102:3336-3341, and Robertson, K.D. (2005) Nature Review Genetics 6:597-610, the disclosures of which are incorporated herein by reference.

The effective amount of an agent that is sufficient to decrease the amount of genomic DNA methylation in a cell may also be determined by assaying for changes in the expression of methylation-sensitive genes in the cell. Methylation-sensitive genes are genes whose expression levels are sensitive to the methylation state of their promoters.

Increased methylation of CpG sequences in the promoters of some genes may be associated with reduced transcriptional activity of methylation-sensitive gene promoters and reduced expression of methylation-sensitive genes, whereas demethylation of CpG sequences in the promoters of those genes may be associated with increased transcriptional activity of methylation-sensitive gene promoters and increased expression of these genes. An effective amount of an agent that promotes demethylation of a methylation-sensitive gene promoter will induce an increase in the expression of that gene by at least about 2-fold. Changes in the level of gene expression following contact between the cells and an agent that promotes CD activity can be assayed by measuring RNA and/or protein levels of the gene before and after contact of the cell with the agent, by, for example, RT-PCR, Northern blot hybridization, Western blot hybridization or ELISA. Methylation-sensitive genes are well known in the art, and include such genes as, for example, those recited in the preceeding paragraph.

### Cells

Cells suitable for use in the methods of the invention may be any mammalian cell, including humans, primates, domestic and farm animals, and zoo, laboratory or pet animals, such as dogs, cats, cattle, horses, sheep, pigs, goats, rabbits, rats, mice etc. In aspects of the invention drawn to increasing the amount of genomic DNA demethylation activity in a cell, the cells are preferably demethylation-permissive cells. Demethylation-permissive cells are cells that are capable of having their CpG sequences converted from methylated CpG sequence to unmethylated CpG sequence. One can determine if a cell is permissive to demethylation by overexpressing a cDNA encoding Activation-induced Cytidine Deaminase (AID) in the cell, providing the cell with a vector carrying CpG-rich DNA, and harvesting and analyzing the exogenously supplied CpG-rich DNA by, for example, bisulphate sequencing or methylase-specific restriction endonuclease digestion, for the extent of methylation.

In the case where a cell of interest for use in the method is determined to be not permissive to demethylation, i.e. demethylation-impermissive, the cell may be induced to become demethylation-permissive cell by contacting the demethylation-impermissive cell with an effective amount of one or more agents that promote the conversion of methylated cytosine to hydroxylated methyl cytosine, one or more agents that promote G:T mismatch-specific repair activity, and/or one or more agents that promote growth arrest and DNA-damage-inducible 45 (GADD45) activity.

An agent that promotes the conversion of methylated cytosine to hydroxylated methyl cytosine will prime methylated nucleic acids for deamination. Examples of agents that promote the conversion of methylated cytosine to hydroxylated methyl cytosine are polypeptides and fragments of tet proteins, i.e. tet1 (Genbank Accession No: NM_{—}030625.2; SEQ ID NO:29 and SEQ ID NO:30), and tet2 (Genbank Accession No: NM_{—}001127208.1 SEQ ID NO:31 and SEQ ID NO:32 (isoform a); and Genbank Accession No: NM_{—}017628.3, SEQ ID NO:33 and SEQ ID NO:34 (isoform b)), and the nucleic acids that encode these polypeptides.

An agent that promotes G:T mismatch-specific repair activity is an agent that promotes the removal of thymine moieties from G/T mismatches and the replacement of these thymine moieties with cytosine moieties. Examples of agents that promote G:T mismatch-specific repair activity are polypeptides and fragments of methyl binding domain proteins (also known as a methyl-Cpg binding domain polypeptides) and the protein thymine-DNA glycosylase (TDG), and the nucleic acids that encode these polypeptides.

Methyl binding domain proteins are nuclear proteins related by the presence in each of a methyl-CpG binding domain. There are five members of this class of proteins: MECP2, MBD1, MBD2, MBD3, and MBD4. Of particular interest are those members with protein sequence similarity to bacterial DNA repair enzymes, as they can function in DNA repair at methyl CpG sites, e.g. MBD4. MBD4 polypeptides and the nucleic acids that encode them that find use in inducing cells to become permissive to demethylation are polypeptides comprising an amino acid sequence that is at least 70% identical to the amino acid sequence of human MBD4, also known as MED1, the sequence of which may be found at GenBank Accession No. NM_{—}003925.1 (SEQ ID NO:35 and SEQ ID NO:36).

The thymine-DNA glycosylase (TDG) protein is an enzyme that plays a central role in cellular defense against genetic mutation caused by the spontaneous deamination of 5-methylcytosine and cytosine, by removing thymine moieties from G/T mismatches and uracil and 5-bromouracil moieties from mispairings with guanine. TDG polypeptides and the nucleic acids that encode them that find use in inducing cells to become permissive to demethyation are polypeptides comprising an amino acid sequence that is at least 70% identical to the amino acid sequence of human TDG, the sequence of which may be found at GenBank Accession No. NM_{—}003211.4 (SEQ ID NO:37 and SEQ ID NO:38).

Growth arrest and DNA-damage-inducible 45 (GADD45) proteins are proteins whose levels are increased following stressful growth arrest conditions and treatment with DNA-damaging agents. GADD45 polypeptides and the nucleic acids that encode them that find use in inducing cells to become permissive to demethylation are polypeptides comprising an amino acid sequence that is at least 70% identical to the amino acid sequence of human GADD45α (GenBank Accession No. NM_{—}001924.2 (SEQ ID NO:39 and SEQ ID NO:40), GADD45β (GenBank Accession No. NM_{—}015675.2 (SEQ ID NO:41 and SEQ ID NO:42), or GADD45γ (GenBank Accession No. NM_{—}006705.3 (SEQ ID NO:43 and SEQ ID NO:44).

Agent(s) that promote G:T mismatch-specific repair activity and agent(s) that promote GADD45 activity can be provided as polypeptides or nucleic acids that encode those polypeptides by methods described above for providing agents that promote CD activity. Cells can be induced to become permissive for demethylation by the methods described above concurrently with contacting the cell with the one or more agents that promote cytidine deaminase activity. Alternatively, the cells can be made permissive for demethylation first, and then contacted with the one or more agents that promote CD activity.

### In Vitro Methods and Uses

In some methods of the invention, the cell is contacted in vitro with the one or more agents that promote CD activity. Demethylation-permissive mammalian cells, and mammalian cells that can be induced to be demethylation-permissive, of interest in these embodiments include pluripotent stem cells, e.g. ES cells, iPS cells, embryonic germ cells; somatic cells, e.g. fibroblasts, hematopoietic cells, neurons, muscle cells, bone cells, vascular endothelial cells, gut cells, and the like, and their lineage-restricted progenitors and precursors; and heterokaryons, which are fusions of two or more types of cells as is well-known in the art and described in the examples below. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The subject cells may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, and other differentiated tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Cells contacted in vitro with the one or more agents that promote cytidine deaminase activity may be incubated in the presence of the agent(s) for about 30 minutes to about 24 hours, e.g., 1 hours, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further.

In some methods of the invention, the demethylation-permissive cell that is contacted with the agent that promotes CD activity is a demethylation-permissive somatic cell. In some of these methods, the demethylation-permissive somatic cell is reprogrammed to become a somatic cell of a different cell lineage. In other words, methods of the invention may be used to promote the conversion of somatic cells of one lineage to somatic cells of another lineage. Somatic cells of different lineages are readily identifiable by markers and morphologies that are well-known in the art.

In some methods in which a demethylation-permissive somatic cell is contacted with an agent that promotes CD activity, the demethylation-permissive somatic cell is reprogrammed to become an induced pluripotent stem (iPS) cell. In other words, the cell that is produced is an iPS cell. As discussed above, iPS cells are pluripotent stem cells that, have an ES cell-like morphology (e.g. growing as flat colonies with large nucleo-cytoplasmic ratios, defined borders and prominent nuclei) but that are derived from somatic cells.

In some methods of the invention, the demethylation-permissive cell that is contacted with the agent that promotes CD activity is a pluripotent stem cell, e.g. an embryonic stem (ES) cell, an embryonic germ (EG) cell, or an induced pluripotent stem (iPS) cell. In these methods, the demethylation-permissive pluripotent stem cell is reprogrammed to become a somatic cell. In other words, methods of the invention may be used to promote the programming of pluripotent stem cells to somatic cells. Examples of somatic cells include any differentiated cells from ectodermal (e.g., neurons and fibroblasts), mesodermal (e.g., cardiomyocytes), or endodermal (e.g., pancreatic cells) lineages. The somatic cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, cardiomyocytes, and the like. As indicated above, the somatic cells derived from the pluripotent stem cells may be terminally differentiated cells, or they may be capable of giving rise to cells of a specific lineage. For example, pluripotent cells can be differentiated into a variety of multipotent cell types, e.g., neural stem cells, cardiac stem cells, or hepatic stem cells. The stem cells may then be further differentiated into new cell types, e.g., neural stem cells may be differentiated into neurons; cardiac stem cells may be differentiated into cardiomyocytes; and hepatic stem cells may be differentiated into hepatocytes. The somatic cells that are produced by such methods are readily identifiable as such by markers and morphologies of particular cell-lineages that are well-known in the art, as described above.

To promote reprogramming of demethylation-permissive cells into other types of cells, an additional step of contacting the demethylation-permissive cell with one or more agents that promote cell reprogramming may be performed. This step may be executed prior to contacting the demethylation-permissive cells with the agent that promotes CD activity, concurrently with contacting the demethylation-permissive cells with the agent that promotes CD activity, or subsequent to contacting the demethylation-permissive cells with the agent that promotes CD activity. The agents that promote cell reprogramming may be polypeptides, nucleic acid agents, or small molecule agents. Examples of agents that may be provided in this step include, but are not limited to, GSK-3 inhibitors, e.g. CHIR99021 and the like (Li, W. et al. (2009) Stem Cells, Epub Oct. 16 2009); HDAC inhibitors, e.g. Valproic Acid and the like (Huangfu, D. (2008) Nature Biotechnol 26(7):795-797; and as described in US20090191159, the disclosure of which is incorporated herein by reference); histone methyltransferase inhibitors, e.g. G9a histone methyltransferase inhibitors, e.g. BIX-01294, and the like (Shi, Y et al. (2008) Cell Stem Cel 3(5):568-574); agonists of the dihydropyridine receptor, e.g. BayK8644, and the like (Shi, Y et al. (2008) Cell Stem Cell 3(5):568-574); and inhibitors of TGFβ signaling, e.g. RepSox and the like (Ichida, J K. et al. (2009) Cell Stem Cell 5(5):491-503). Other examples of agents that may be provided in this step include reprogramming factors. As discussed above, reprogramming factors are biologically active factors that act on a cell to alter transcription, thereby reprogramming a cell to a new cell fate.

Numerous examples of agents that promote reprogramming of somatic cells of one cell lineage into somatic cells of another cell lineage are known in the art, any of which may find use in the present invention. These include, for example, the reprogramming factors MYOD (Myogenic factor 1; Genbank Accession Nos. NM_{—}002478.4 and NP_{—}002469.2), which induces muscle-specific properties in pigment, nerve, fat. liver and fibroblasts, see, e.g., Weintraub, H. W. et al. Proc. Natl. Acad. Sci. USA 86:5434-5438; Davis, R. L., et al. (1987) Cell 51:987-1000; Schafer, B. W., et al. (1990) Nature 344:454-8); NEUROG3 (neurogenin3, NGN3; Genbank Accession Nos. NM_{—}020999.2 and NP_{—}066279.2), PDX1 (pancreatic and duodenal homeobox 1; Genbank Accession Nos. NM_{—}000209.3 and NP_{—}000200.1) and MafA (v-maf musculoaponeurotic fibrosarcoma oncogene homolog A; Genbank Accession Nos. NM_{—}201589.2 and NP_{—}963883.2), which in combination can efficiently convert pancreatic exocrine cells into functional 6-cells in vivo, see, e.g., Zhou, Q., et al. (2008) Nature 455:627-32); and C/EBPα (CCAAT/enhancer binding protein, alpha; Genbank Accession Nos. NM_{—}004364.3 and NP_{—}004355.2), which induces macrophage characteristics either alone in B-cells or in combination with Pu.1 (spleen focus forming virus (SFFV) proviral integration oncogene, SPI1; Genbank Accession No. NM_{—}001080547.1, NP_{—}001074016.1, NM_{—}003120.2 and NP_{—}003111.2) in fibroblasts, see, e.g., Bussmann, L. H. et al. (2009) Cell Stem Cell 5:554-66; Feng, R. et al. (2008) Proc Natl Acad Sci USA 105: 6057-62; Xie, H., et al. (2004) Cell 117:663-76). Other agents include the IL2 receptor (IL receptor 2A and IL receptor 2B; Genbank Accession Nos. NM_{—}000417.2, NP_{—}000408.1, NM_{—}000878.2 and NP_{—}000869.1) and GM-CSF receptor (colony stimulating factor 2 receptor, alpha (CSF2RA) and colony stimulating factor 2 receptor, beta (CSF2RB); Genbank Accession Nos. NM_{—}001161529.1, NP_{—}001155001.1, NM_{—}000395.2, and NP_{—}000386.1), which induce myeloid conversion in committed lymphoid progenitor cells, see, e.g., Kondo, M. et al. (2000) Nature 407:383-6). Polypeptides comprising an amino acid sequence that is at least 70%, 75%, 80%, 85%, 90%, 91%, 92%, 95%, 97%, 99%, or 100% identical to the amino acid sequence of the agents discussed above as described in the Genbank Accession Numbers recited above, as well as the nucleic acids that encode these polypeptides, find use as agents that promote reprogramming of demethylation-permissive somatic cells of one cell lineage into somatic cells of another cell lineage in the methods of the invention.

Numerous examples of agents that promote reprogramming of somatic cells into iPS cells are known in the art, any of which may find use in the present invention. see, e.g. US Application Nos. 20090047263, US20090068742, US20090191159, US20090227032, US20090246875, and US20090304646, the disclosures of which are incorporated herein by reference, These include, for example, the reprogramming factors Oct3/4, (POU class 5 homeobox 1 (POU5F1); GenBank Accession Nos. NP_{—}002692 and NM_{—}002701); Sox2 (sex-determining region Y-box 2 protein; GenBank Accession Nos. NP_{—}003097 and NM_{—}003106): Klf4 (Kruppel-Like Factor 4; GenBank Accession Nos. NP 004226 and NM_{—}004235); c-Myc (myelocytomatosis viral oncogene homolog; GenBank Accession Nos. NP_{—}002458 and NM_{—}002467); Nanog (Nanog homeobox; GenBank Accession Nos. NP_{—}079141 and NM_{—}024865); and Lin-28 (Lin-28 homolog of C. elegans; GenBank Accession Nos. NP_{—}078950 and NM_{—}024674). Polypeptides comprising an amino acid sequence that is at least 70%, 75%, 80%, 85%, 90%, 91%, 92%, 95%, 97%, 99%, or 100% identical to the amino acid sequence of the agents discussed above as described in the Genbank Accession Numbers recited above, as well as the nucleic acids that encode these polypeptides, find use as agents that promote reprogramming of demethylation-permissive somatic cells into iPS cells in the methods of the invention.

Numerous examples of agents that promote reprogramming of pluripotent stem cells into somatic cells are known in the art, any of which may find use in the present invention. For example, neural stem cells may be generated by culturing the pluripotent cells as floating aggregates in the presence of NOG (noggin; GenBank Accession Nos. NM_{—}005450.4 and NP_{—}005441.1) or other bone morphogenetic protein antagonist (Itsykson et al., (2005), Mol, Cell Neurosci., 30(1):24-36) or by culturing the pluripotent cells in suspension to form aggregates in the presence of growth factors, e.g., FGF-2 (fibroblast growth factor 2, also known as basic fibroblast growth factor (bFGF); GenBank Accession Nos. NM_{—}002006.4 and NP_{—}001997.5), see, e.g., Zhang et al., (2001), Nat. Biotech., (19): 1129-1133. In some cases, the aggregates are cultured in serum-free medium containing FGF-2. In another example, the pluripotent cells are co-cultured with a mouse stromal cell line, e.g., PA6 in the presence of serum-free medium comprising FGF-2. In yet another example, the pluripotent cells are directly transferred to serum-free medium containing FGF-2 to directly induce differentiation.

Neural stems derived from the pluripotent cells may be differentiated into neurons, oligodendrocytes, or astrocytes. Often, the conditions used to generate neural stem cells can also be used to generate neurons, oligodendrocytes, or astrocytes. For example, to promote differentiation into dopaminergic neurons, pluripotent cells or the neural stem cells derived therefrom may be co-cultured with a PA6 mouse stromal cell line under serum-free conditions, see, e.g., Kawasaki et al., (2000) Neuron, 28(1):3140. Other methods have also been described, see, e.g., Pomp et al., (2005), Stem Cells 23(7):923-30; U.S. Pat. No. 6,395,546, e.g., Lee et al., (2000), Nature Biotechnol., 18:675-679. Differentiation of the pluripotent cells or the neural stem cells derived therefrom into oligodendrocytes may be promoted by, e.g. co-culturing pluripotent cells or neural stem cells with stromal cells, see, e.g., Hermann et al. (2004), J Cell Sci. 117(Pt 19):4411-22, or by culturing the pluripotent cells or neural stem cells in the presence of a fusion protein, in which the Interleukin (IL)-6 receptor (GenBank Accession Nos. NM_{—}000565.2 and NP_{—}000556.1), or a derivative thereof, is linked to the IL-6 cytokine (GenBank Accession Nos. NM_{—}000600.3 and NP_{—}000591.1), or derivative thereof. Oligodendrocytes can also be generated from the pluripotent cells by other methods known in the art, see, e.g. Kang et al., (2007) Stem Cells 25, 419-424. Astrocytes may also be produced from the pluripotent cells or the neural stem cells derived therefrom by, e.g. culturing pluripotent cells or neural stem cells in the presence of neurogenic medium with bFGF and EGF (epidermal growth factor; GenBank Accession Nos. NM_{—}001963.3 and NP_{—}001954.2), see e.g., Brustle et al., (1999), Science, 285:754-756.

Pluripotent cells may be differentiated into pancreatic beta cells by methods known in the art, e.g., Lumelsky et al., (2001) Science, 292:1389-1394; Assady et al., (2001), Diabetes, 50:1691-1697; D'Amour et al., (2006), Nat. Biotechnol., 24:1392-1401; D'Amour et al., (2005), Nat. Biotechnol. 23:1534-1541. The method may comprise culturing the pluripotent cells in serum-free medium supplemented with Activin A (inhibin, beta A (INHBA); GenBank Accession Nos. NM_{—}002192.2 and NP_{—}002183.1), followed by culturing in the presence of serum-free medium supplemented with all-trans retinoic acid, followed by culturing in the presence of serum-free medium supplemented with bFGF and nicotinamide, e.g., Jiang et al., (2007), Cell Res., 4:333-444. In other examples, the method comprises culturing the pluripotent cells in the presence of serum-free medium, activin A, and Wnt protein (e.g. GenBank Accession Nos. NM_{—}005430, NM_{—}003391, NM_{—}004185, NM_{—}030753, NM_{—}033131, NM_{—}030761, NM_{—}003392, NM_{—}032642, NM_{—}006522, NM_{—}004625, NM_{—}058238, NM_{—}058244, NM_{—}003393, NM_{—}003395, NM_{—}003396, NM_{—}025216, NM_{—}003394, Wnt-11 NM_{—}004626, and NM_{—}016087). from about 0.5 to about 6 days, e.g., about 0.5, 1, 2, 3, 4, 5, 6, days; followed by culturing in the presence of from about 0.1% to about 2%, e.g., 0.2%, FBS and activin A from about 1 to about 4 days, e.g., about 1, 2, 3, or 4 days; followed by culturing in the presence of 2% FBS, FGF10 (fibroblast growth factor 10, GenBank Accession Nos. NM_{—}004465.1 and NP_{—}004456.1), KAAD-cyclopamine (keto-N-aminoethylaminocaproyl dihydro cinnamoylcyclopamine) and retinoic acid from about 1 to about 5 days, e.g., 1, 2, 3, 4, or 5 days; followed by culturing with 1% B27, gamma secretase inhibitor and extendin-4 from about 1 to about 4 days, e.g., 1, 2, 3, or 4 days; and finally culturing in the presence of 1% B27, extendin-4, IGF-1, and HGF for from about 1 to about 4 days, e.g., 1, 2, 3, or 4 days.

Hepatic cells or hepatic stem cells may be differentiated from the pluripotent cells. For example, culturing the pluripotent cells in the presence of sodium butyrate may generate hepatocytes, see e.g., Rambhatla et al., (2003), Cell Transplant 12:1-11. In another example, hepatocytes may be produced by culturing the pluripotent cells in serum-free medium in the presence of Activin A, followed by culturing the cells in FGF4 (fibroblast growth factor-4; GenBank Accession Nos. NM_{—}002007.2 and NP_{—}001998.1) and BMP2 (bone morphogenetic protein-2; GenBank Accession Nos. NM_{—}001200.2 and NP_{—}001191.1), e.g., Cai et al., (2007) Hepatology 45(5): 1229-39. In an exemplary embodiment, the pluripotent cells are differentiated into hepatic cells or hepatic stem cells by culturing the pluripotent cells in the presence of Activin A from about 2 to about 6 days, e.g., about 2, about 3, about 4, about 5, or about 6 days, and then culturing the pluripotent cells in the presence of HGF (hepatocyte growth factor; GenBank Accession Nos. NM_{—}010427.4 and NP_{—}034557.3) for from about 5 days to about 10 days, e.g., about 5, about 6, about 7, about 8, about 9, or about 10 days.

The pluripotent cells may also be differentiated into cardiac muscle cells. Inhibition of bone morphogenetic protein (BMP) signaling may result in the generation of cardiac muscle cells (or cardiomyocytes), see, e.g., Yuasa et al., (2005), Nat. Biotechnol., 23(5):607-11. Thus, in an exemplary embodiment, the pluripotent cells are cultured in the presence of NOG (noggin) for from about two to about six days, e.g., about 2, about 3, about 4, about 5, or about 6 days, prior to allowing formation of an embryoid body, and culturing the embryoid body for from about 1 week to about 4 weeks, e.g., about 1, about 2, about 3, or about 4 weeks. In other examples, cardiomyocytes may be generated by culturing the pluripotent cells in the presence of LIF (leukemia inhibitory factor; GenBank Accession Nos. NM_{—}002309.3 and NP_{—}002300.1), or by subjecting them to other methods known in the art to generate cardiomyocytes from ES cells, e.g., Bader et al., (2000), Circ. Res., 86:787-794, Kehat et al., (2001), J. Clin. Invest., 108:407-414; Mummery et al., (2003), Circulation, 107:2733-2740.

Examples of methods to generate other cell-types from pluripotent cells include: (1) culturing pluripotent cells in the presence of retinoic acid, LIF, thyroid hormone, and insulin in order to generate adipocytes, e.g., Dani et al., (1997), J. Cell Sci., 110:1279-1285; (2) culturing pluripotent cells in the presence of BMP2 or BMP4 (GenBank Accession Nos. NM_{—}001202.3, NP_{—}001193.2, NM_{—}130850.2, NP_{—}570911.2, NM_{—}130851.2, and NP_{—}570912.2) to generate chondrocytes, e.g., Kramer et al., (2000), Mech. Dev., 92:193-205; (3) culturing the pluripotent cells under conditions to generate smooth muscle, e.g., Yamashita et al., (2000), Nature, 408:92-96; (4) culturing the pluripotent cells in the presence of beta-1 integrin (GenBank Accession Nos. NM_{—}002211.3 and NP_{—}002202.2) to generate keratinocytes, e.g., Bagutti et al., (1996), Dev. Biol., 179:184-196; (5) culturing the pluripotent cells in the presence of IL3 (Interleukin-3; GenBank Accession Nos. NM_{—}000588.3 and NP_{—}000579.2) and CSF1 (colony stimulating factor, macrophage; GenBank Accession Nos. NM_{—}000757.4, NP_{—}000748.3) to generate macrophages, e.g., Lieschke and Dunn (1995), Exp. Hemat., 23:328-334; (6) culturing the pluripotent cells in the presence of IL-3 and SCF (stem cell factor also known as steel factor, kit ligand; GenBank Accession Nos. NM_{—}000899.3 and NP_{—}000890.1) to generate mast cells, e.g., Tsai et al., (2000), Proc. Natl. Acad. Sci. USA, 97:9186-9190; (7) culturing the pluripotent cells in the presence of dexamethasone and SCF to generate melanocytes, e.g., Yamane et al., (1999), Dev. Dyn., 216:450-458; (8) co-culturing the pluripotent cells with fetal mouse osteoblasts in the presence of dexamethasone, retinoic acid, ascorbic acid, beta-glycerophosphate to generate osteoblasts, e.g., Buttery et al., (2001), Tissue Eng., 7:89-99; (9) culturing the pluripotent cells in the presence of osteogenic factors to generate osteoblasts, e.g., Sottile et al., (2003), Cloning Stem Cells, 5:149-155; (10) overexpressing insulin-like growth factor-2 in the pluripotent cells and culturing the cells in the presence of dimethyl sulfoxide to generate skeletal muscle cells, see, e.g., Prelle et al., (2000), Biochem. Biophys. Res. Commun., 277:631-638; (11) subjecting the pluripotent cells to conditions for generating white blood cells; or (12) culturing the pluripotent cells in the presence of BMP4 and one or more: SCF, FLT3 (fms-related tyrosine kinase 3; GenBank Accession Nos. NM_{—}004119.2 and NP_{—}004110.2), IL-3, IL-6 (interleukin 6; GenBank Accession Nos. M_{—}000600.3 and NP_{—}000591.1), and CSF3 (colony stimulating factor, granulocyte; GenBank Accession Nos. NM_{—}000759.2 and NP_{—}000750.1) to generate hematopoietic progenitor cells, see, e.g., Chadwick et al., (2003), Blood, 102:906-915.

Polypeptides comprising an amino acid sequence that is at least 70%, 75%, 80%, 85%, 90%, 91%, 92%, 95%, 97%, 99%, or 100% identical to the amino acid sequence of the agents discussed above as described in the Genbank Accession Numbers recited above, as well as the nucleic acids that encode these polypeptides, find use as agents that promote reprogramming of pluripotent cells into somatic cells in methods of the invention.

The agents that promote cell reprogramming may be provided to the demethylation-permissive cells by methods that are well-known in the art including but not limited to those described above for agents that promote CD activity. Agents may be provided individually or as a single composition, that is, as a premixed composition, of agents. The agents may be added to the subject cells simultaneously or sequentially at different times. In some embodiments, a set of at least two agents is provided, e.g. an Oct3/4 polypeptide and a Sox2 polypeptide. In some embodiments, a set of three agents is provided, e.g., an Oct3/4 polypeptide, a Sox2 polypeptide, and a Klf4 polypeptide. In some embodiments, a set of four agents is provided e.g., an Oct3/4 polypeptide, a Sox2 polypeptide, a Klf4 polypeptide, and a c-Myc polypeptide. As with the agent(s) that promote CD activity, the agent(s) may be provided to the subject cells one or more times and the cells allowed to incubate with the agents for some amount of time following each contacting event, e.g. 16-24 hours, after which time the media is replaced with fresh media and the cells are cultured further.

After contacting the demethylation-permissive cells with the agent(s) that promote CD activity, the contacted cells are cultured so as to promote the outgrowth of the desired cells. Methods for culturing cells to promote the growth of iPS cells or particular types of somatic cells as described above, for isolating iPS cell clones or clones of particular types of somatic cells as described above, and for culturing cells of those cell clones so as to promote the outgrowth of iPS cells or of particular types of somatic cells as described above are well known in the art, any of which may be used in the present invention to grow, isolate and reculture the desired cells from the reprogrammed demethylation-permissive cells.

Decreasing the amount of genomic DNA methylation in cells of a demethylation-permissive cell culture by contacting the cells with agent(s) that promote CD activity increases the efficiency of reprogramming those demethylation-permissive cells to the desired cell type relative to the efficiency observed in the absence of the agents that promote CD activity. In other words, somatic cells and cell cultures demonstrate an enhanced ability to give rise to the desired type of cell when contacted with one or more agents that promote CD activity in the presence of factors known in the art to promote reprogramming relative to cells that were not contacted with the one or more agents that promote CD activity. By enhanced, it is meant that the somatic cell cultures have the ability to give rise to the desired cell type that is at least about 50%, about 100%, about 200%, about 300%, about 400%, about 600%, about 1000%, at least about 2000% of the ability of the population of cells that were not contacted with the agent that promotes CD activity. In other words, the culture of demethylation-permissive cells produces about 1.5 fold, about 2-fold, about 3-fold, about 4-fold, about 6-fold, about 10-fold, about 20-fold, about 30-fold, about 50-fold, about 100-fold, about 200-fold the number of cells of the desired cell type that are produced by a population of demethylation-permissive cells that are not contacted with the one or more agents that promote CD activity. The efficiency of reprogramming may be determined by assaying the amount of methylation at promoters known in the art to become demethylated upon the acquisition of the desired cell type. In such cases, an enhanced efficiency of reprogramming due to the presence of an agent that promotes CD activity is observed when the amount of methylation at those promoters is about 1.5 fold, about 2-fold, about 3-fold, about 4-fold, about 6-fold, about 10-fold less than the amount of methylation observed in the absence of the agent that promotes CD activity. Alternatively or additionally, the efficiency of reprogramming may be determined by assaying the level of expression of gene known in the art to become more highly expressed upon the acquisition of the desired cell type. In such cases, an enhanced efficiency of reprogramming due to the presence of an agent that promotes CD activity is observed when the level of expression of these genes is about 1.5 fold, about 2-fold, about 3-fold, about 4-fold, about 6-fold, about 10-fold greater than the level of expression observed in the absence of the agent that promotes CD activity.

Cells derived from demethylation-permissive cells reprogrammed by the above in vitro methods may be used as a therapy to treat disease (e.g., a genetic defect). Specifically, somatic cells derived from demethylation-permissive somatic cells by the methods above and somatic cells derived from pluripotent stem cells by the methods above may be transferred to subjects suffering from a wide range of diseases or disorders, for example to reconstitute or supplement differentiating or differentiated cells in a recipient. Likewise, induced pluripotent stem cells derived from demethylation-permissive somatic cells may be transferred to subjects suffering from a wide range of diseases or disorders, or they may be differentiated into somatic cells of various cell lineages in vitro and then transferred to subjects suffering from a wide range of diseases or disorders. There are numerous methods of differentiating the pluripotent cells into a more specialized cell type, including but not limited to methods of differentiating pluripotent cells may used to reprogram stem cells, particularly ES cells, to become somatic cells as described above.

The therapy may be directed at treating the cause of the disease; or alternatively, the therapy may be to treat the effects of the disease or condition. For example, the derived cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

In some cases, the derived cells or a sub-population of derived cells may be purified or isolated prior to transferring to the subject. In some cases, one or more monoclonal antibodies specific to the desired cell type are incubated with the cell population and those bound cells are isolated. In other cases, the desired subpopulation of cells expresses a reporter gene that is under the control of a cell type specific promoter, which is then used to purify or isolate the derived cells or a subpopulation thereof.

In some cases, genes may be introduced into the demethylation-permissive cells or the cells derived therefrom prior to transferring to a subject for a variety of purposes, e.g. to replace genes having a loss of function mutation, provide marker genes, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

To prove that one has genetically modified the demethylation-permissive cells or the cells derived thereform, various techniques may be employed. The genome of the cells may be restricted and used with or without amplification. The polymerase chain reaction; gel electrophoresis; restriction analysis; Southern, Northern, and Western blots; sequencing; or the like, may all be employed. The cells may be grown under various conditions to ensure that the cells are capable of maturation to all of the myeloid lineages while maintaining the ability to express the introduced DNA. Various tests in vitro and in vivo may be employed to ensure that the pluripotent capability of the cells has been maintained.

The number of administrations of treatment to a subject may vary. Introducing the induced and/or differentiated cells into the subject may be a one-time event; but in certain situations, such treatment may elicit improvement for a limited period of time and require an on-going series of repeated treatments. In other situations, multiple administrations of the cells may be required before an effect is observed. The exact protocols depend upon the disease or condition, the stage of the disease and parameters of the individual subject being treated.

The cells may be introduced to the subject via any of the following routes: parenteral, intravenous, intraarterial, intramuscular, subcutaneous, transdermal, intratracheal, intraperitoneal, or into spinal fluid.

Subjects suffering from neurological diseases or disorders could especially benefit from therapies that utilize cells derived by the methods of the invention. In some approaches, neural stem cells or neural cells may be transplanted to an injured site to treat a neurological condition, e.g., Alzheimer's disease, Parkinson's disease, multiple sclerosis, cerebral infarction, spinal cord injury, or other central nervous system disorder, see, e.g., Morizane et al., (2008), Cell Tissue Res., 331(1):323-326; Coutts and Keirstead (2008), Exp. Neurol., 209(2):368-377; Goswami and Rao (2007), Drugs, 10(10):713-719. For the treatment of Parkinson's disease, dopamine-acting neurons may be transplanted into the striate body of a subject with Parkinson's disease. For the treatment of multiple sclerosis, oligodendrocytes or progenitors of oligodendrocytes may be transferred to a subject suffering from MS. The cells derived by the methods of the invention may also be engineered to respond to cues that can target their migration into lesions for brain and spinal cord repair, e.g., Chen et al., (2007), Stem Cell Rev., 3(4):280-288.

Diseases other then neurological disorders may also be treated by therapies that utilize cells generated by the methods of the invention. Degenerative heart diseases such as ischemic cardiomyopathy, conduction disease, and congenital defects could benefit from the transplantation of cardiomyocytes or their precursors, see, e.g. Janssens et al., (2006), Lancet, 367:113-121.

Pancreatic islet cells (or primary cells of the islets of Langerhans) may be transplanted into a subject suffering from diabetes (e.g., diabetes mellitus, type 1), see e.g., Burns et al., (2006) Curr. Stem Cell Res. Ther., 2:255-266. In some embodiments, pancreatic beta cells derived by methods of the invention may be transplanted into a subject suffering from diabetes (e.g., diabetes mellitus, type 1).

In other examples, hepatic cells or hepatic stem cells derived by methods of the invention are transplanted into a subject suffering from a liver disease, e.g., hepatitis, cirrhosis, or liver failure.

Hematopoietic cells or hematopoietic stem cells (HSCs) derived by methods of the invention may be transplanted into a subject suffering from cancer of the blood, or other blood or immune disorder. Examples of cancers of the blood that are potentially treated by hematopoietic cells or HSCs include: acute lymphoblastic leukemia, acute myeloblastic leukemia, chronic myelogenous leukemia (CML), Hodgkin's disease, multiple myeloma, and non-Hodgkin's lymphoma. Often, a subject suffering from such disease must undergo radiation and/or chemotherapeutic treatment in order to kill rapidly dividing blood cells. Introducing HSCs derived by the methods of the invention to these subjects may help to repopulate depleted reservoirs of cells.

In some cases, hematopoietic cells or HSCs derived by the methods of the invention may also be used to directly fight cancer. For example, transplantation of allogeneic HSCs has shown promise in the treatment of kidney cancer, see, e.g., Childs et al., (2000), N. Engl. J. Med., 343:750-758. In some embodiments, allogeneic, or even autologous, HSCs derived by the methods of the invention may be introduced into a subject in order to treat kidney or other cancers.

Hematopoietic cells or HSCs derived by the methods of the invention may also be introduced into a subject in order to generate or repair cells or tissue other than blood cells, e.g., muscle, blood vessels, or bone. Such treatments may be useful for a multitude of disorders.

In some cases, the cells derived by the methods of the invention are transferred into an immunocompromised animal, e.g., SCID mouse, and allowed to differentiate. The transplanted cells may form a mixture of differentiated cell types and tumor cells. The specific differentiated cell types of interest can be selected and purified away from the tumor cells by use of lineage specific markers, e.g., by fluorescent activated cell sorting (FACS) or other sorting method, e.g., magnetic activated cell sorting (MACS). The differentiated cells may then be transplanted into a subject (e.g., an autologous subject, HLA-matched subject) to treat a disease or condition. The disease or condition may be a hematopoietic disorder, an endocrine deficiency, degenerative neurologic disorder, hair loss, or other disease or condition described herein.

The cells derived by the methods of the invention may be administered in any physiologically acceptable medium. They may be provided alone or with a suitable substrate or matrix, e.g. to support their growth and/or organization in the tissue to which they are being transplanted. Usually, at least 1×10⁵ cells will be administered, preferably 1×10⁶ or more. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with progenitor cell proliferation and differentiation.

### In Vivo Methods and Uses

In some embodiments, the demethylation-permissive cell is contacted in vivo with the one or more agents that promote CD activity, e.g. in a subject in need of genomic DNA demethylation therapy.

Cells in vivo may be contacted with agent(s) that promote CD activity by any of a number of well-known methods in the art for the administration of polypeptides, small molecules and nucleic acids to a subject. The agent can be incorporated into a variety of formulations. More particularly, the agent can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the Agent(s) that promote cytidine deaminase activity can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

For some conditions, particularly central nervous system conditions, it may be necessary to formulate agents to cross the blood brain barrier (BBB). One strategy for drug delivery through the blood brain barrier (BBB) entails disruption of the BBB, either by osmotic means such as mannitol or leukotrienes, or biochemically by the use of vasoactive substances such as bradykinin. The potential for using BBB opening to target specific agents to brain tumors is also an option. A BBB disrupting agent can be co-administered with the therapeutic compositions of the invention when the compositions are administered by intravascular injection. Other strategies to go through the BBB may entail the use of endogenous transport systems, including caveoil-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to the therapeutic compounds for use in the invention to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

The calculation of the effective amount or effective dose of agent(s) that promote CD activity to be administered is within the skill of one of ordinary skill in the art, and will be routine to those persons skilled in the art. Needless to say, the final amount to be administered will be dependent upon the route of administration and upon the nature of the disorder or condition that is to be treated.

For inclusion in a medicament, agent(s) that promote CD activity may be obtained from a suitable commercial source. As a general proposition, the total pharmaceutically effective amount of the compound administered parenterally per dose will be in a range that can be measured by a dose response curve.

Agent(s) that promote CD activity to be used for therapeutic administration must be sterile. Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The agent(s) that promote CD activity ordinarily will be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD₅₀ animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Mammalian species that may be treated with the present methods include canines and felines; equines; bovines; ovines; etc. and primates, particularly humans. Animal models, particularly small mammals, e.g. murine, lagomorpha, etc. may be used for experimental investigations. Other uses include investigations where it is desirable to investigate a specific effect in the presence of active demethylation signaling.

The methods of the present invention also find use in combined therapies. For example, a number of agents may be useful in the treatment of cancer, e.g. chemotherapeutic agents, kinase inhibitors, angiostatin, endostatin, VEGF inhibitors, etc. The combined use of agent(s) that promote CD activity of the present invention and these other agents may have the advantages that the required dosages for the individual drugs is lower, and the effect of the different drugs complementary.

As mentioned above, the present invention finds use in the treatment of mammals, such as human patients, in subjects in need of genomic DNA demethylation therapy. Examples of such subjects would be subjects suffering from conditions associated with aberrantly silenced genes due to hypermethylation of their promoters. Patients suffering from diseases characterized by such conditions will benefit greatly by a treatment protocol of the pending claimed invention.

One example of such a condition is cancer. A number of genes, i.e. methylation-sensitive genes, are known to be aberrantly hypermethylated and silenced in cancer. These include genes involved in cell cycle regulation (e.g. RB1, CDKN2A^{INK4A}, CDKN2A^{ARF}), tumor cell invasion (e.g. CDH1, CDH13, TIMP3, VHL), DNA repair (e.g. MLH1, MGMT, BRCA1, GSTP1), chromatin remodeling (e.g. SMARCA3), cell signaling (e.g. RASSF1A, SOCS1), transcription (e.g. ESR1), and apoptosis (e.g. DAPK1). Accordingly, methods and compositions of the present invention find use in inhibiting tumor growth and the progression of cancer in a subject suffering from cancer, e.g. gliomas, medulloblastomas, colon cancer, colorectal cancer, breast cancer, or leukemia. The term “cancer” refers to the physiological condition in mammals that is typically characterized by unregulated cell growth/proliferation. Examples of cancer include, but are not limited to: carcinoma, lymphoma, blastoma, and leukemia. More particular examples of cancers include, but are not limited to: chronic lymphocytic leukemia (CLL), lung, including non small cell (NSCLC), breast, ovarian, cervical, endometrial, prostate, colorectal, intestinal carcinoid, bladder, gastric, pancreatic, hepatic (hepatocellular), hepatoblastoma, esophageal, pulmonary adenocarcinoma, mesothelioma, synovial sarcoma, osteosarcoma, head and neck squamous cell carcinoma, juvenile nasopharyngeal angiofibromas, liposarcoma, thyroid, melanoma, basal cell carcinoma (BCC), medulloblastoma and desmoid. Correlations between particular cancers and the methylation status of the above genes of interest may be found in Robertson, K.D. (2005) Nature Review Genetics 6:597-610, the disclosure of which is incorporated herein by reference.

An effective amount of an agent(s) that promote CD activity to inhibit tumor growth and cancer progression is the amount that will increase, e.g. by 2-fold or more, the expression of one or more of the aforementioned methylation-sensitive genes in vitro and in vivo, and/or which result in measurable reduction in the rate of proliferation of cancer cells in vitro or growth inhibition of a tumor in vivo. For example, preferred growth inhibitory agents will inhibit growth of tumor by at least about 5%, at least about 10%, at least about 20%, preferably from about 20% to about 50%, and even more preferably, by greater than 50% (e.g., from about 50% to about 100%) as compared to the appropriate control, the control typically being cancer cells not treated with the agent(s) that promote cytidine deaminase activity being tested. An agent is growth inhibitory in vivo if administration of the agent at about 1 μg/kg to about 100 mg/kg body weight results in reduction in tumor size or cell proliferation within about 5 days to 3 months from the first administration of the antibody, preferably within about 5 to 30 days. In a specific aspect, the tumor size is reduced relative to its size at the start of therapy.

Another example of a condition associated with aberrantly silenced genes due to hypermethylation of their promoters that may be treated by the methods of the invention are conditions associated with aberrant genomic imprinting. In genomic imprinting, certain genes are expressed in a parent-of-origin-specific manner. It is an inheritance process independent of the classical Mendelian inheritance, in which imprinted genes are either expressed only from the allele inherited from the mother or from the allele inherited from the father. Genomic imprinting involves methylation and histone modifications in order to achieve monoallelic gene expression without altering the genetic sequence. These epigenetic marks are established in the germline and are maintained throughout all somatic cells of an organism.

A number of conditions have been identified that are associated with aberrant genomic imprinting that would be amenable to treatment by methods of the invention. For example, in Beckwith-Wiedemann syndrome, which is characterized by fetal and postnatal overgrowth, enlarged organs, increased risk of tumors, and facial abnormalities, de novo methylation of the maternal allele at the IGF2/H19 imprinting control region 1 is observed. In Prader-Willi syndrome, which is characterized by mental retardation, obesity, short stature, and behavioural problems, de novo methylation of the paternal allele of the PWS gene is observed. In Pseudohypoparathyroidism type 1B, characterized by renal parathyroid hormone resistance, de novo methylation of the maternal allele of NESP55 is observed. Methods of the present invention find use in promoting demethylation at these loci, thereby restoring appropriate gene expression.

Another example of a condition associated with aberrantly silenced genes due to hypermethylation of their promoters that may be treated by the methods of the invention is a condition associated with a repeat instability disease. In these diseases, expansion of repeat sequences results in aberrant methylation that affects the expression of genes near those sequences. A number of conditions have been identified that are associated with repeat instability that would be amenable to treatment by methods of the invention. For example, in Fragile X syndrome, which is characterized by mental retardation, macro-orchidism, and autistic behavior, the expansion of a CGG repeat in the 5′UTR of FMRI gene results in de novo methylation of the 5′ UTR sequence and aberrant silencing of the FMRI gene. As another example, in Myotonic Dystrophy (DM1), which is characterized by weakness and wasting of limb and facial muscles, myotonia, and cataracts, the expansion of a CTG repeat in the UTR of the DMPK gene results in de novo methylation of CpG islands near the expanded CTG repeat, which in turn disrupts and silences the SIX5 gene. Methods of the present invention find use in promoting demethylation at these loci, thereby restoring appropriate gene expression.

### Screening Methods.

The methods described herein provide a useful system for screening candidate agents for activity in modulating demethylation. To that end, it has been shown that agents that promote CD activity have a potent effect on enhancing demethylation. Addition of agents that inhibit CD activity to cell culture systems comprising cells in which demethylation is occurring strongly suppress this demethylation activity, such that the amount of transcriptional activity of promoters of methylation-sensitive genes such as Oct4 and Nanog is reduced. This suppression of demethylation activity and subsequent increase in methylation at these promoters and silencing of transcriptional activity can be observed in as little as one day after contacting demethylating cells with the agents that inhibit CD activity, with an almost complete silencing of these methylation-sensitive genes by day 3.

In screening assays for biologically active agents, cells, usually cultures of cells, are contacted with the agent of interest in the presence of an agent that promotes CD activity, and the effect of the candidate agent is assessed by monitoring output parameters, such as the amount of methylated CpG sequences, the expression of methylation-sensitive genes, and the like, by methods described above.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

For example, agents can be screened for an activity in promoting demethylation activity, e.g. by adding the candidate agent to a cell culture in the presence of an agent that promotes CD activity. A decrease in the amount of methylation observed, e.g. a 1.5-fold, a 2-fold, a 3-fold or more decrease in the number of 5-methylcytosines, e.g. of the promoter of a methylation-sensitive gene or an exogenously supplied 5-meCpG-rich nucleic acid, over that observed in the culture absent the candidate agent would indicate that the candidate agent was an agent that promotes demethylation. In such embodiments, the cell may be a demethylation-permissive cell, or it may be a demethylation-impermissive cell.

Alternatively, agents can be screened for an activity in suppressing demethylation activity, e.g. by adding the candidate agent to a cell culture in the presence of an agent that promotes CD activity. No decrease or a decrease of only small amounts in the amount of methylation observed, e.g. in the number of 5-methylcytosines, e.g. of the promoter of a methylation-sensitive gene or an exogenously supplied 5-meCpG-rich nucleic acid, relative to that observed in the culture absent the candidate agent would indicate that the candidate agent was an agent that suppresses demethylation. In such embodiments, the cells of the culture are demethylation-permissive cells.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA, antisense molecules, or miRNA, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc. Vectors may be provided directly to the subject cells. In other words, the pluripotent cells are contacted with vectors comprising the nucleic acid of interest such that the vectors are taken up by the cells.

Methods for contacting cells with nucleic acid vectors, such as electroporation, calcium chloride transfection, and lipofection, are well known in the art. Alternatively, the nucleic acid of interest may be provided to the subject cells via a virus. In other words, the pluripotent cells are contacted with viral particles comprising the nucleic acid of interest. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid encoding the reprogramming factors into packaging cell lines and of collecting the viral particles that are generated by the packaging lines are well known in the art.

Vectors used for providing nucleic acid of interest to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced from eukaryotic produced by prokaryotic cells, it may be further processed by unfolding, e.g. heat denaturation, DTT reduction, etc. and may be further refolded, using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For example, for measuring the state of DNA methylation, e.g. at a particular CpG sequence, Chromatin immunoprecipitation (ChIP) can be performed to isolate endogenous DNA, which can then be digested with restriction endonuclease HpaII to determine the extent of demethylation, or bisulphate sequencing can be performed. For measuring the amount of a molecule that is present, e.g. when measuring expression of methylation-sensitive genes, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81).

Screens such as those described above can be tailored to identify agents that have an activity in modulating demethylation in particular biological systems. For example, agents that promote demethylation of the promoters of methylation-sensitive genes such as genes that regulate the cell cycle, tumor-cell invasion, DNA repair, chromatin remodeling, cell signaling, transcription and apoptosis in tumor cells may find use in promoting demethylation of these genes and hence, expression of these genes in a tumor, thereby preventing cancer cell proliferation and tumor growth. As another example, agents that promote demethylation at the promoters of methylation-sensitive genes such as the pluripotency genes Oct4 and Nanog in somatic cells or heterokaryons between ES cells and somatic cells may find use in promoting demethylation of genes associated with pluripotency in known methods for producing iPS cells. In some such cases, e.g. somatic cells, these methods may include a step of providing the cells with reprogramming factors so as to further promote the iPS phenotype for screening purposes.

Kits may be provided, where the kit will comprise one or more agents that promote CD activity and reagents to induce cells to be demethylation-permissive as described herein. A combination of interest may include one or more AID or APOBEC polypeptides or vectors comprising nucleic acids encoding those peptides and one or more agents that promote reprogramming. Kits may further include reagents suitable for determining the methylation state of DNA in subject cells. Kits may also include tubes, buffers, etc., and instructions for use.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

To identify novel early regulators essential to nuclear reprogramming towards pluripotency, we capitalized on our previous experience with heterokaryons that proved useful in elucidating the principles inherent to the maintenance of the differentiated state of somatic cells. Specifically, these earlier studies by us and others showed that the “terminally differentiated” state of human cells was not fixed, but could be altered and the expression of previously silent genes typical of other differentiated states induced (Blau, H. M., et al. (1983) Cell 32, 1171-801; Baron, M. H. & Maniatis, T. (1986) Cell 46, 591-602; Wright, W. E. (1984) Exp Cell Res 151, 55-69; Spear, B. T. & Tilghman, S. M. (1990) Mol Cell Biot 10, 5047-54; Chiu, C. P. & Blau, H. M. R (1984) Cell 37, 879-87). We reasoned that heterokaryons could be used to elucidate mechanisms and identify novel genes with a role at the onset of reprogramming towards pluripotency because: (1) reprogramming takes place in the presence of all ES cell factors, (2) the onset of reprogramming is synchronously initiated upon fusion, (3) reprogramming is assessed in fused, non-dividing cells, and (4) species differences distinguish the transcripts of the fused cell types.

### Materials and Methods

Heterokaryon Generation and Isolation by Flow Cytometry.

GFP+ murine ES cells and DsRed+ human fetal lung primary fibroblasts were generated by transduction with retroviral constructs as previously described (Palermo, A. et al. (2009) Faseb J), and fused to form non-dividing, multinucleated heterokaryons. Cells were first co-cultured for 12 h in ES media and then treated with PEG 1500 (Roche) for 2 min at 37° C., followed by four successive washes with DMEM. ES media was replaced after washing and every 12 h thereafter. GFP+/DsRed+ heterokaryons were sorted twice by flow-cytometry (FACSVantage SE, BD) and analyzed for gene expression and methylation.

Immunofluorescence.

Heterokaryons were sorted twice in PBS with 2.5% v/v goat serum and 1 mM EDTA, and cytospun at 900 rpm for 5 min. The cytospun GFP+/DsRed+ heterokaryons were stained with Hoechst 33342, and imaged. For antibody staining, cytospun cells were fixed, permeabilized and blocked using 20% FBS in PBS. Cells were incubated with the primary antibody mouse anti-Ki-67 (Dako Denmark A/S) at 1:100 dilution in blocking buffer for 1 h, rinsed 3 times in PBS, and then incubated with a goat anti-mouse Cascade blue secondary antibody (Millipore) at 1:500 dilution for 30 min, rinsed 3 times and mounted with Fluoromount-G and imaged. Images were acquired using an epifluorescent microscope (Axioplan2; Carl Zeiss Microlmaging, Inc.), Fluar 20×/0.75 or 40×/0.90 objective lens, and a digital camera (ORCA-ER C4742-95; Hamamatsu Photonics). The software used for acquisition was OpenLab 4.0.2 (Improvision).

BrdU was added to mES and hFb co-cultures 3 hours after PEG-induced fusion. Labeling and antibody staining was performed using the BrdU Labeling and Detection Kit I (Roche).

Analysis of Gene Expression.

RNA was prepared from ES cells, fibroblasts and twice-sorted heterokaryons at different times post fusion or after siRNA treatment using the RNeasy micro kit (Qiagen). Total RNA for each sample was reverse transcribed using the Superscript First-Strand Synthesis System for RT-PCR (Invitrogen). The reverse transcribed material was subjected to PCR using Go GreenTaq DNA polymerase (Promega). Human specific primers were designed for analyzing the expression of Oct4, Nanog and GAPDH. Primers used for AID and GAPDH in the siRNA treatment experiments amplify both human and mouse transcripts to assess the total levels of AID and GAPDH in heterokaryons. Human-specific primers used for RT-PCR and quantitative PCR are: hOct4 F 5′-TCGAGAACCGAGTGAGAGGC-3′ (SEQ ID NO:45), R-5′-CACACTCGGACCACATCCTTC-3′ (SEQ ID NO:46); hNanog F 5′-CCAACATCCTGAACCTCAGCTAC-3′ (SEQ ID NO:47), R 5′-GCCTTCTGCGTCACACCATT-3′ (SEQ ID NO:48); hGAPDH F 5′-TGTCCCCACTGCCAACGTGTCA-3′ (SEQ ID NO:49), R 5′-AGCGTCAAAGGTGGAGGAGTGGGT-3′ (SEQ ID NO:50). Non-species specific primer sequences for assessing knockdown after siRNA treatment are as follows: GAPDH F 5′-ACCACAGTCCATGCCATCAC-3′ (SEQ ID NO:51), R 5′-TCCACCACCCTGTTGCTGTA-3′ (SEQ ID NO:52); AID F 5′-AAAATGTCCGCTGGGCTAAG-3′ (SEQ ID NO:53), R 5′-AGGTCCCAGTCCGAGATGTAG-3′ (SEQ ID NO:54).

Real Time PCR.

Real time PCR was performed using an ABI 7900HT Real time PCR system using the Sybr Green PCR mix (Applied Biosystems). Samples were cycled at 94° C. for 2 min, 40× (94° C. for 20 s, 58° C. for 45 s).

TABLE 1
Human-specific primers used for real time PCR

| Gene | primer | SEQ ID NO: |
|---|---|---|
| Essrb 5′ | GCCAGCGCCATGAGGAGC | 55 |
| Essrb 3′ | GTATCCAGCCTGAGCAGTGC | 56 |
| TDGF1 5′ | ATTGCCATTTTCGCTTTAGG | 57 |
| TDGF1 3′ | ACACGCTGGGAAGACCGAGGC | 58 |
| Sox2 5′ | CGACACCCCCGCCCGCCT | 59 |
| Sox2 3′ | ACACCATGAAGGCATTCATGGGCC | 60 |
| Klf4 5′ | ACCCCGACCCTGGGTCTT | 61 |
| Klf4 3′ | GCCACTGACTCCGGAGGA | 62 |
| c-myc 5′ | AAGGGAGATCCGGAGCGAATA | 63 |
| c-myc 3′ | GGAGGCTGCTGGTTTTCCACT | 64 |

Single cell RT-PCR.

Single heterokaryons were directly sorted by FACS (FACSVantage SE, BD) into PCR tubes containing 9-μl aliquots of RT-PCR lysis buffer. The buffer components included commercial RT-PCR buffer (SuperScript One-Step RT-PCR Kit Reaction Buffer, Invitrogen), RNase inhibitor (Protector RNase Inhibitor, Roche) and 0.15% IGEPAL detergent (Sigma). After a short pulse-spin, the PCR-tubes were immediately shock-frozen and stored at −80° C. for subsequent analysis.

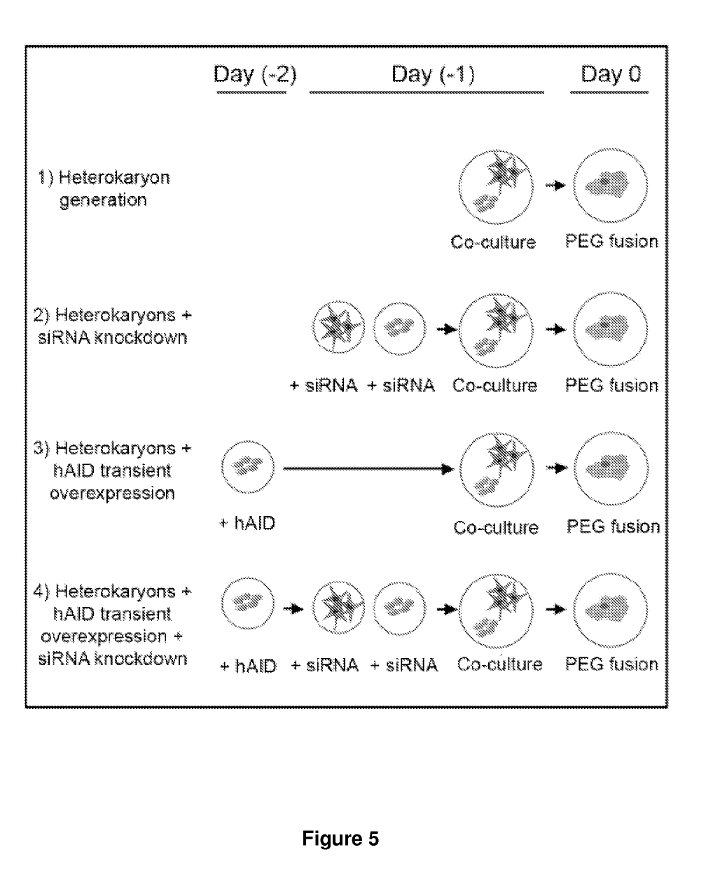

For two-step multiplex nested single cell RT-PCR, cell lysates were first reverse-transcribed using the human and gene-specific primer pairs for Oct4, Nanog and GAPDH (Table 2, External primers; FIG. 5b) using SuperScript One-Step RT-PCR Kit (Invitrogen). Briefly, the RT-PCR was performed in the same PCR cell-lysis tubes by addition of an RT-PCR-reaction mix containing the genespecific primer pairs and RNase inhibitor. Genomic products were excluded by designing and using intron-spanning primer sets for the first and second round PCR and nested RT-PCR ensured greater specificity. In the first step, the reverse transcription reactions were carried out at 55° C. for 30 min, and followed by a 2-min step at 94° C. Subsequently, 30 cycles of PCR amplification were performed as follows: 94° C. for 30 s; 58° C. for 30 s; 68° C. for 30 s. In the final PCR step, the reactions were incubated for 3 min at 68° C. The completed reactions were stored at 4° C.

In the second step of the PCR protocol, the completed RT-PCR reaction from the first step was diluted 1:1 with water. One percent of these reactions were replica transferred into new reaction tubes for the second round of PCR, which was performed for each of the genes separately using nested gene-specific internal-primers, for greater specificity, in a total reaction volume of 20 μl (Platinum Taq Super-Mix HF, Invitrogen). Thirty cycles of PCR amplification were performed as follows: 94° C. for 30 s; 58° C. for 30 s; 68° C. for 30 s. In the final PCR step, the reactions were incubated for 3 min at 68° C. The completed reactions were stored at 4° C. The second-round PCR products were then subjected to gel electrophoresis using one fifth of the reaction volumes and 1.4% agarose gels.

TABLE 2
Primer sequences utilized for single cell nested PCR in heterokaryons

| | Nested primer set | | | |
|---|---|---|---|---|
| | | SEQ ID | | SEQ ID |
| | External Primer [5′-3′] | NO: | Internal Primer [5′-3′] | NO: |
| Oct4 5′ | GAAGGAGAAGCTGGAGCAAAAC | 65 | GAGAGGCAACCTGGAGAATT | 66 |
| Oct4 3′ | CAAAAACCCTGGCACAAACT | 67 | CCAGAGGAAAGGACACTGGT | 68 |
| Nanog 5′ | TGATTTGTGGGCCTGAAG | 69 | GATGCCTGGTGAACCCGA | 70 |
| Nanog 3′ | AACCAGAACACGTGGTTTCC | 71 | TGCACCAGGTCTGAGTGTTC | 72 |
| GAPDH 5′ | GCTCAGACACCATGGGGAAG | 73 | CCATGAGAAGTATGACAACAGC | 74 |
| GAPDH 3′ | CCATGAGAAGTATGACAACAGC | 75 | TTCTAGACGGCAGGTCAGG | 76 |

DNA Methylation Analyses.

FACS-sorted heterokaryons (2,000-10,000 cells) were collected in 20 uL PBS. DNA was extracted using the DNeasy Tissue Kit (Qiagen). Bisulfite treatment was performed using the Epitect Bisulfite Kit (Qiagen). Nested PCR for regions of the human Oct4 and Nanog promoters was performed using human and bisulfite specific primers (Table 3). Samples were cycled for the first and nested PCR at 94° C. for 2 min, 30× (94° C. for 20 s, 68° C. for 30 s, 68° C. for 30 s). PCR products from second-round bisulfite-specific PCR amplification were cloned and sequenced as described before (Zhang, F., et al. (2007) Proc Natl Acad Sci USA 104, 4395-400).

TABLE 3
Human and bisulfite specific primers for DNA methylation analyses

| | | SEQ ID | | SEQ ID |
|---|---|---|---|---|
| | External Primer [5′-3′] | NO: | Internal Primer [5′-3′] | NO: |
| Oct4 5′ | GAGGAGTTGAGAGGGTGATTGG | 77 | GGAGAGGGGGTTAAGTATTTGG | 78 |
| | TTTT | | GTTTT | |
| Oct4 3′ | CGAAAAAACTACTCAACCCCT | 79 | TCCACTTTATTACCCAAACTAA | 80 |
| Nanog 5′ | GGAAAATGGAGTTAGTTGAAATT | 81 | GGAATTTAAGGTGTATGTATTTT | 82 |
| | TTTGTTT | | | |
| Nanog 3′ | CCACCCCTATAATCCCAATAAAT | 83 | AACCAACCTAACCAACATAA | 84 |
| | TAAAA | | | |
| B globin 5′ | TGATTAAATAAGTTTTAGTTTTTT | 85 | CCATGAGAAGTATGACAACAGC | 86 |
| | TTTAGTTTT | | | |
| B globin 3′ | TAAGTATGAGTAGTTTTGGTTAG | 87 | TTCCATATCCTTATTTCATATTA | 88 |
| | GTTT | | ATACATA | |

siRNA Transfection.

For siRNA transfection, ES cells and primary fibroblasts were plated at 50-60% confluence the day before transfection. siRNAs (Dharmacon) were transfected using silmporter (Millipore).

Chromatin Immunoprecipitation.

Chromatin immunoprecipitation was performed as previously described by Dahl and Collas ((2008) Nat Protoc 3, 1032-45) using primers provided in Table 4. ChIP data was presented as normalized to input DNA and the error bars represent standard error mean (sem).

TABLE 4
Primers used for ChIP experiments

| | |
|---|---|
| primer | SEQ ID NO: |

Human primers [5′-3′]

| | | |
|---|---|---|
| Thy1.1 5′ | TCCCACAGACTCCTGAAGAATA | 89 |
| Thy1.1 3′ | TTGTTCCCCTTTTAAGGCTTT | 90 |
| Nanog 5′ | GAGTACAGTGGCGCGATATCG | 91 |
| Nanog 3′ | CGGGAGAATCCCTTGAACCT | 92 |
| Oct4 5′ | GTGGCTCACGCCTTTAATCA | 93 |
| Oct4 3′ | CCAGGCTGGTCTTGAATTCC | 94 |
| Cμ 5′ | ACCCCAATGCCACTTTCA | 95 |
| Cμ 3′ | AGTCATCCTCGCAGATGCT | 96 |

Mouse primers [5′-3′]

| | | |
|---|---|---|
| Cdx2 5′ | AGGTTAAAGTGCACCCAGGTT | 97 |
| Cdx2 3′ | CAGGCCCTTCTTGCTAGCT | 98 |
| Nanog 5′ | AACGCTGAGTGCTGAAAGGA | 99 |
| Nanog 3′ | GTCAGACCTTGCTGCCAAAG | 100 |
| Oct4 5′ | GGGTGGGTAAGCAAGAACT | 101 |
| Oct4 3′ | AATGTTCGTGTGCCAATTA | 102 |
| p53 5′ | ACGGCAGCTTGCACCTCTA | 103 |
| p53 3′ | CTTTCTAGCAACCCGTTTGC | 104 |

Statistical Analysis.

Data are presented as the mean±s.e.m. Comparisons between groups used the Student's t test assuming two-tailed distributions.

Thy1.1 (CD90) Enrichment of Heterokaryons.

GFP⁻ (non-GFP) mES and DsRed⁺ hFb co-cultures treated with PEG were trypsinized and resuspended in 3 mL FACS buffer. Cells were incubated for 30 min at room temperature with biotin mouse anti-human CD90 (BD Pharmingen) at a dilution of 1:5000. The cells were washed once, resuspended in 3 mL FACS buffer incubated for 30 min at room temperature with 10 uL of Dynabeads Biotin Binder (Invitrogen). Beads were removed by magnetic isolation, washed twice and the enriched heterokaryons were cytospun.

Immunoprecipitation and Western Blots.

Mouse ES cells were lysed in IP buffer (20 mM Tris pH 7.5, 1 mM DTT, 0.5 mM EDTA, 350 mM NaCl, 10% (vol/vol) glycerol, 10 uM ZnCl. Whole cell lysates were pre-cleared for 30 min at room temperature followed by AID pull down using. Briefly, cell lysates and then AID was pulled down using Protein A Plus Agarose beads (Pierce) cross-linked to a rabbit polyclonal AID antibody. Immunoprecipitation was performed from 2 mg of cell lysates.

To visualize AID protein knockdown in mES, cell lysates were harvested 3 days posttransfection with siControl or si-1. Detection of AID in these samples was performed from 170 ug of whole cell lysate using anti mouse-AID (L7E7, Cell Signaling, dilution 1:500). The membrane was stripped and probed with ant-mouse α-tubulin (Sigma, dilution 1:20,000) for the loading control. Immunoprecipitation of AID was detected using the same L7E7 antibody.

### Results

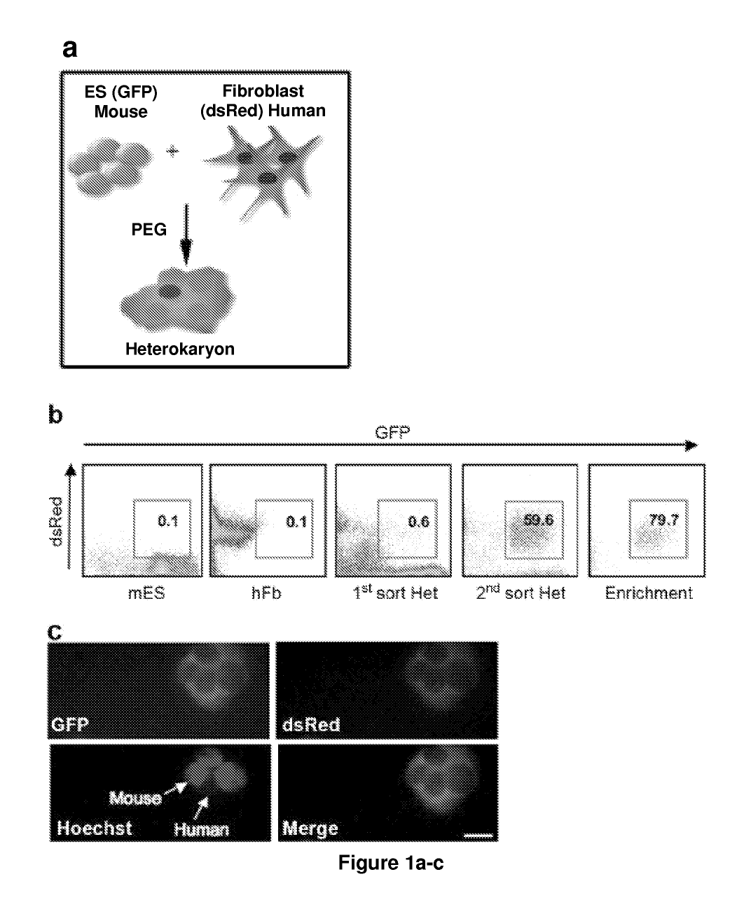

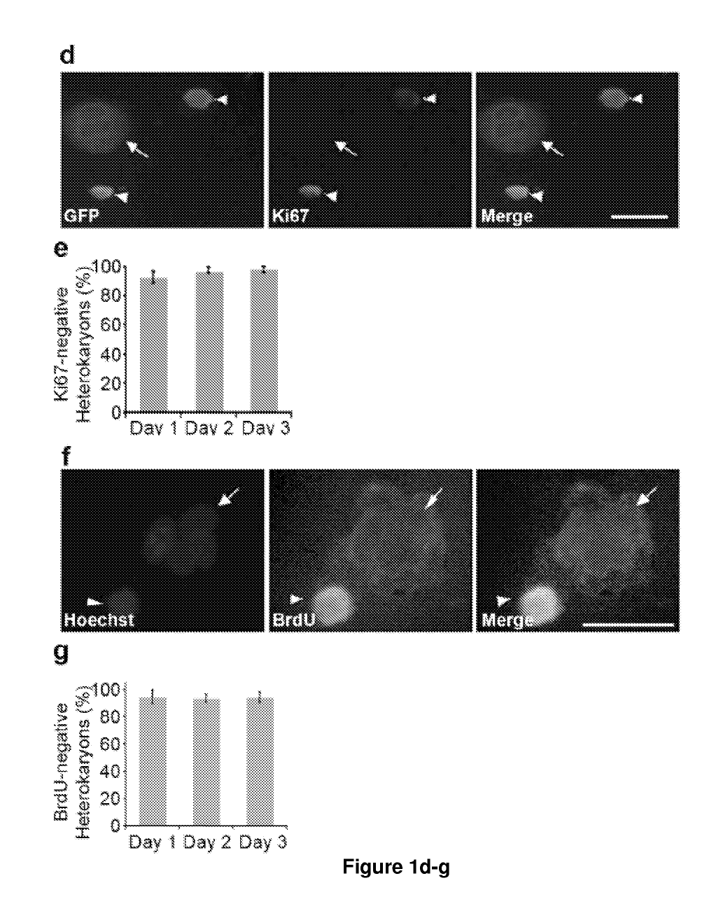

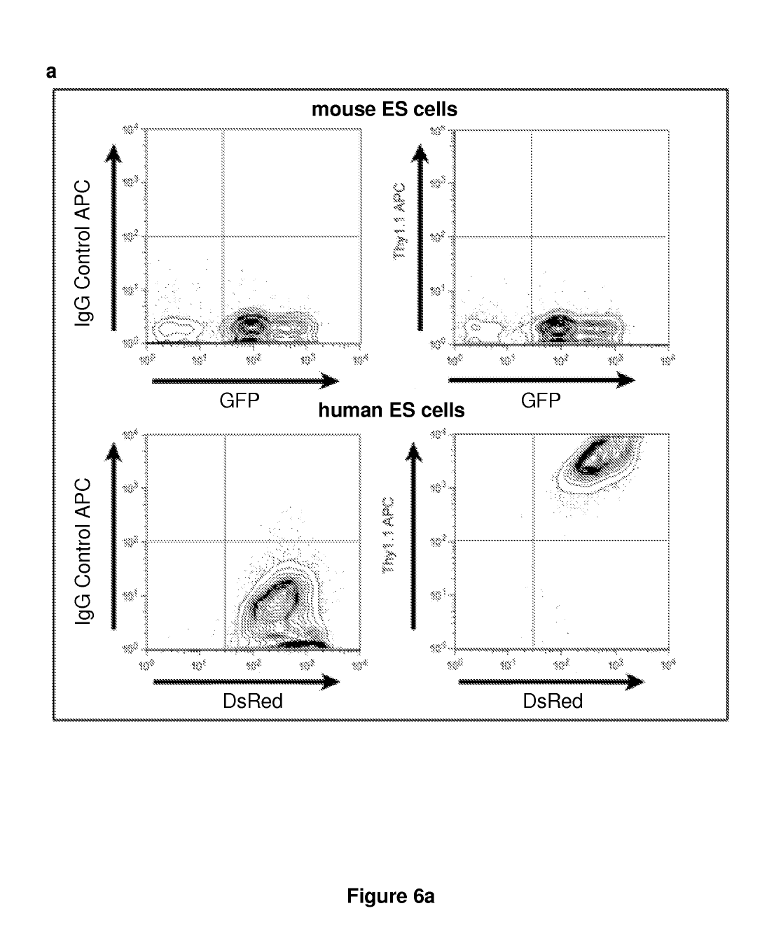

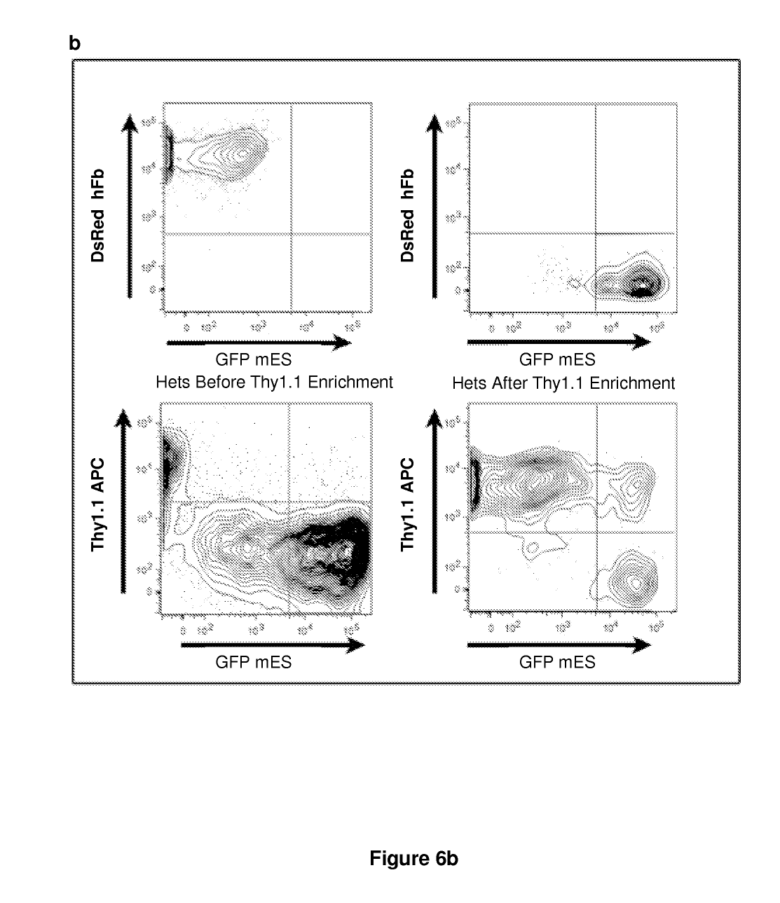

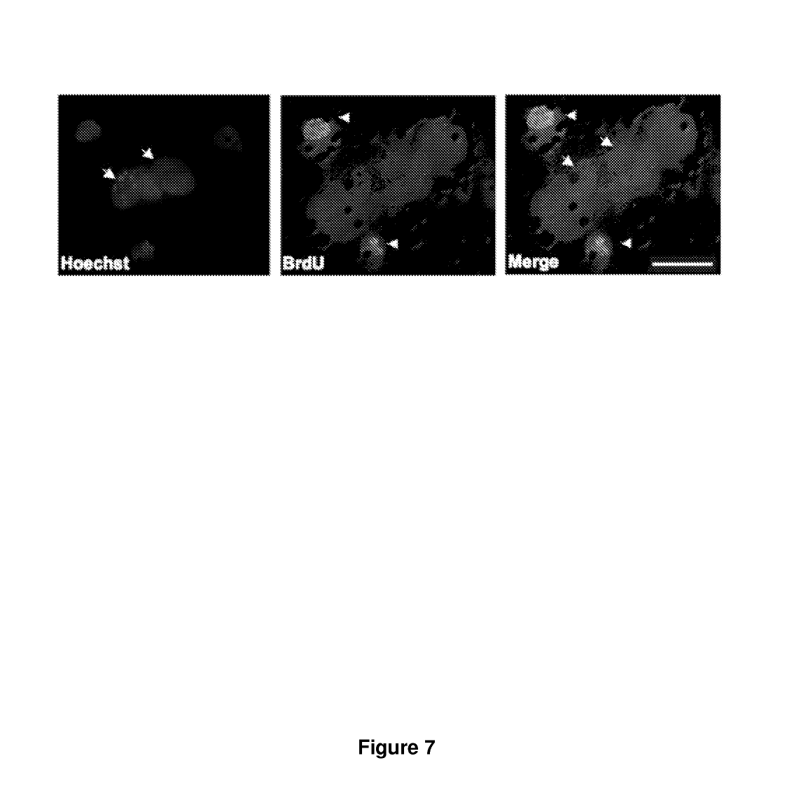

To produce interspecies heterokaryons, mouse embryonic stem cells (mES) transduced with a GFP reporter gene were co-cultured with primary human fibroblasts (hFb) transduced with a DsRed reporter gene, and fused using polyethylene glycol (PEG) (FIG. 1a; Scheme in FIG. 5). Fused GFP+DsRed+ heterokaryons, which were readily sorted by FACS (FIG. 1b) and identified using fluorescence microscopy, contained distinctly stained human and mouse nuclei when visualized with Hoechst 33342 or Hoechst 33258 (FIGS. 1c and 1f, respectively). Since the efficiency of PEG fusion is low (0.6 to 1.0%), GFP⁺DsRed⁺ heterokaryons were sorted twice and enriched to 80% purity (FIG. 1b). Using an antibody for Ki-67, a nuclear protein present only in proliferating cells, we determined that cell division did not occur in 98(±2) % of heterokaryons over the three day time period assayed post fusion (FIG. 1d,e). In addition, BrdU labeling was not detected in 94(±4) % of heterokaryons over the same time period, indicating that DNA replication did not occur (FIG. 1f,g; FIG. 6; FIG. 7). To favor reprogramming towards a pluripotent state, we skewed the ratio of the input cells so that ES cells outnumbered the fibroblasts (2:1), as gene dosage and the proportion of proteins contributed by each cell type determines the direction of nuclear reprogramming in somatic cells

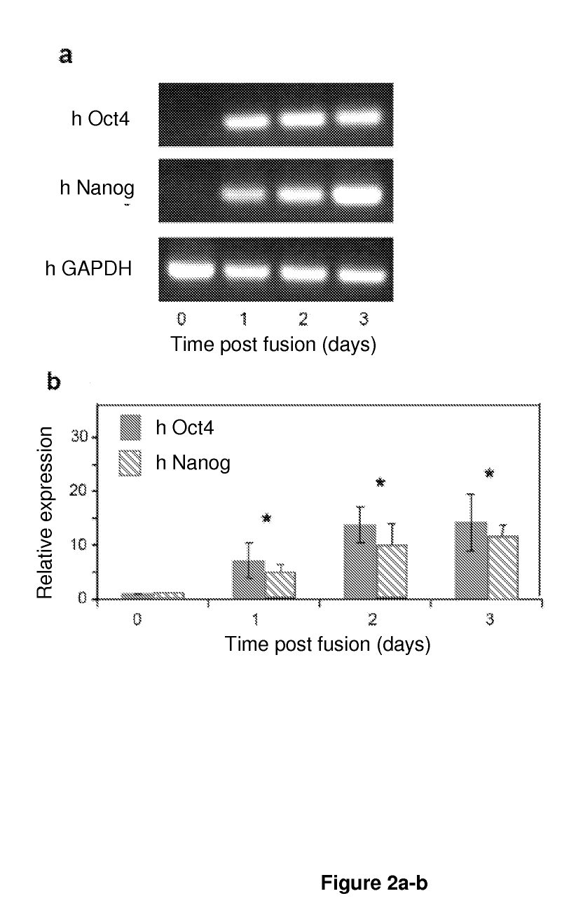

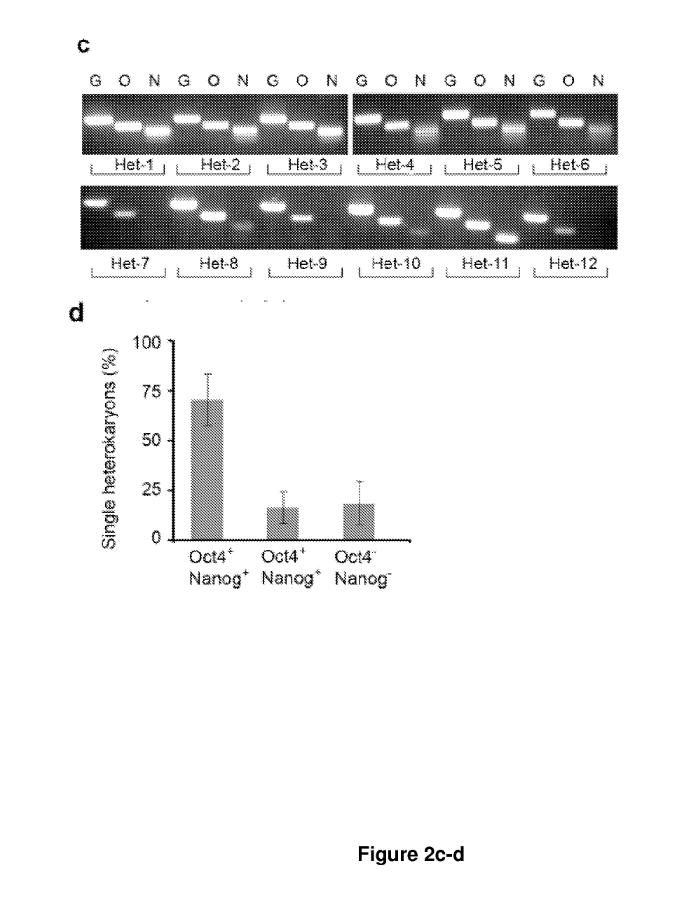

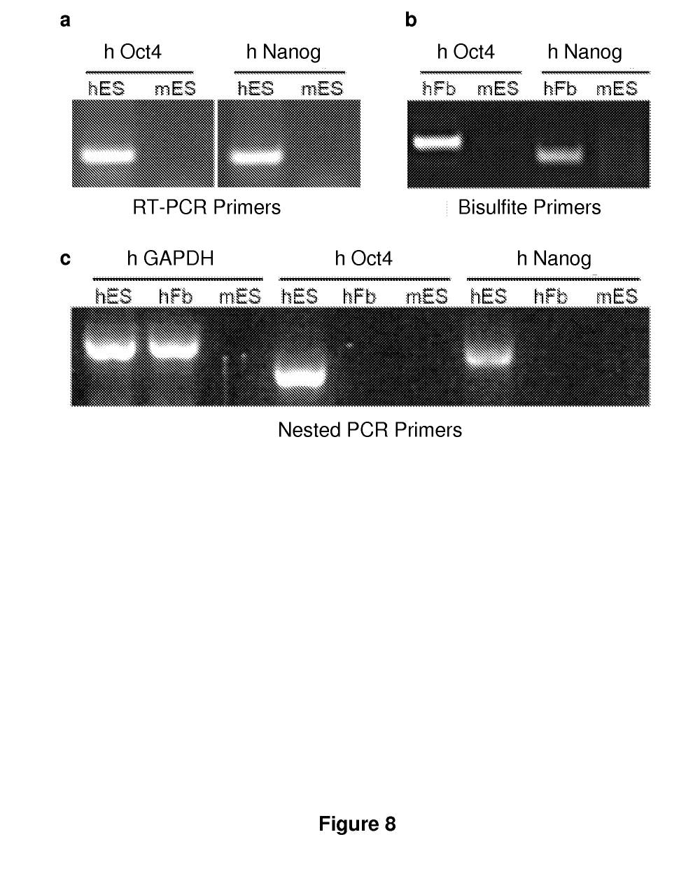

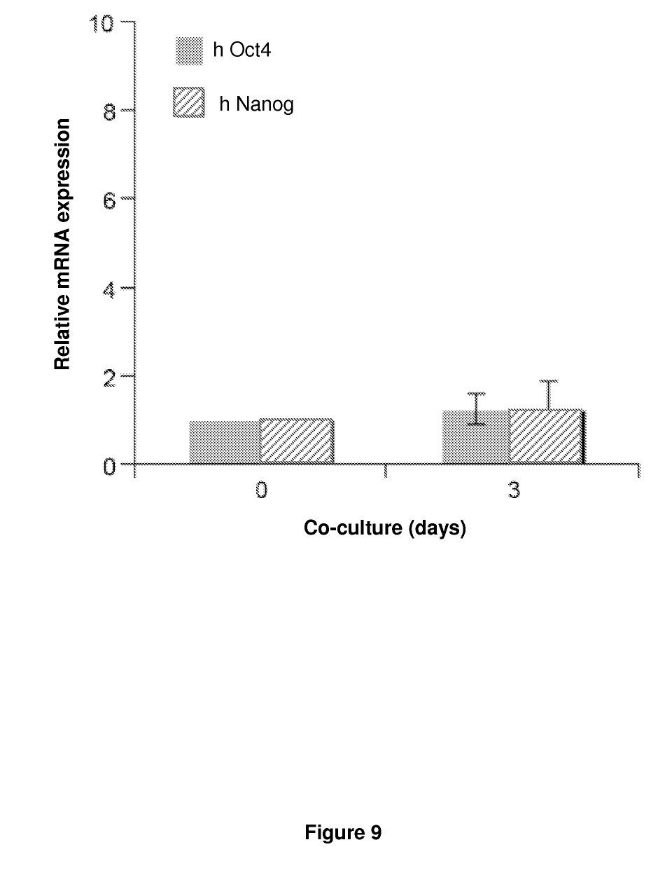

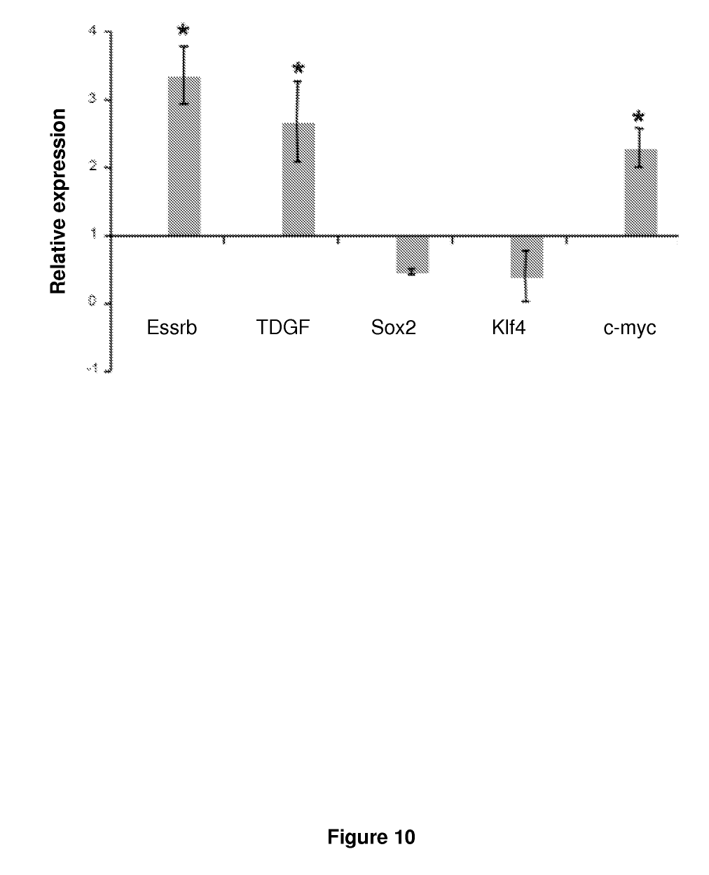

To determine if ES cell-specific genes were induced in the human fibroblasts, the induction of human Oct4 and Nanog were assayed relative to ubiquitous GAPDH using species-specific primers (FIG. 8). mRNA isolated from sorted heterokaryons 1, 2 and 3 days post fusion was assessed by semi-quantitative RT-PCR and real time PCR (FIG. 2a,b). The day 0 controls used were either (a) human fibroblasts alone; (b) pre-PEG, unfused co-cultures of mES and hFb; or (c) human fibroblasts treated with PEG to control for the effects of PEG and fusion. All of the above day 0 controls gave similar results. Induction of both human Oct4 and Nanog transcripts was evident as early as day 1 post fusion in heterokaryons (FIG. 2a,b), but not in controls (FIG. 9), indicating that the onset of expression of two key human pluripotency genes is rapid in heterokaryons. By day 1, expression of human Oct4 and Nanog (normalized to GAPDH) in the same samples, had increased 5-fold relative to the unfused co-culture control (day 0) and persisted at 10-fold on days 2 and 3 (FIG. 2b). Human-specific primers were used to determine if other key pluripotency genes in addition to Oct4 and Nanog were induced using real time PCR. Essrb (Bhattacharya, B. et al. (2004) Blood 103, 2956-64) and TDGF1 (Bhattacharya, B. et al. (2004) Blood 103, 2956-64) (Cripto), which have been shown to be essential for maintaining ES cell self-renewal and are targets of Oct4 and Nanog were found to be upregulated 3-fold and 2.5-fold, respectively, in heterokaryons on day 2 post fusion (FIG. 10). Sox2 is already expressed in human fibroblasts and its promoter is extensively demethylated pre-fusion, in agreement with findings in mouse fibroblasts; its expression did not increase post fusion. Expression of Klf4 (Feng, B. et al. (2009) Nat Cell Biol 11, 197-203), which is functionally interchangeable with Essrb, did not change in heterokaryons at day 2 post fusion (FIG. 10).

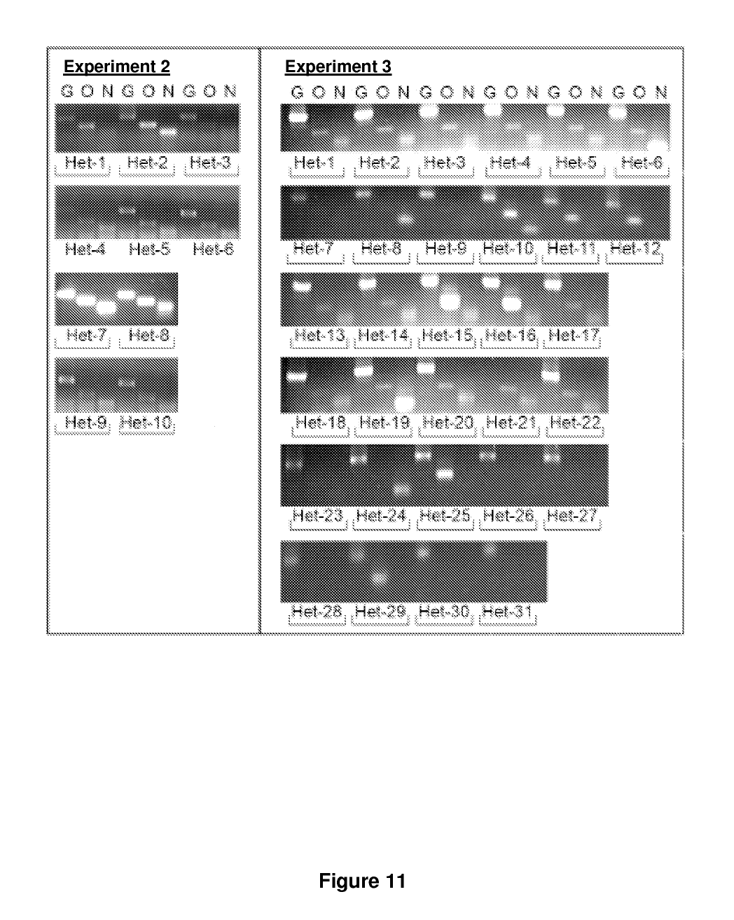

To assess the efficiency of nuclear reprogramming in human fibroblasts following fusion, single FACS-sorted heterokaryons were analyzed by nested RT-PCR for the three human transcripts, Oct4, Nanog, and GAPDH (control), using two sets of human-specific primers in each case (FIG. 2c). No human gene products were detected in mouse ES cells (control) and only human GAPDH was detected in human fibroblasts (control) (FIG. 8). In contrast, 70% of single FACS-sorted heterokaryons from three independent fusion experiments on day 3 post fusion expressed both human Oct4 and Nanog (FIG. 2c,d; FIG. 11), showing that a high proportion of heterokaryons initiated reprogramming towards pluripotency. This is in marked contrast to the slow and inefficient induction of Oct4 and Nanog expression in iPS cells (<0.1%) of the total population in 2 to 3 weeks as observed in, for example, Takahashi, K. et al. (2007) Cell 131, 861-72; Takahashi, K. & Yamanaka, S. (2006) Cell 126, 663-76; Wernig, M. et al. (2007) Nature 448, 318-24; and Wernig, M. et al. (2008) Nat. Biotechnol.

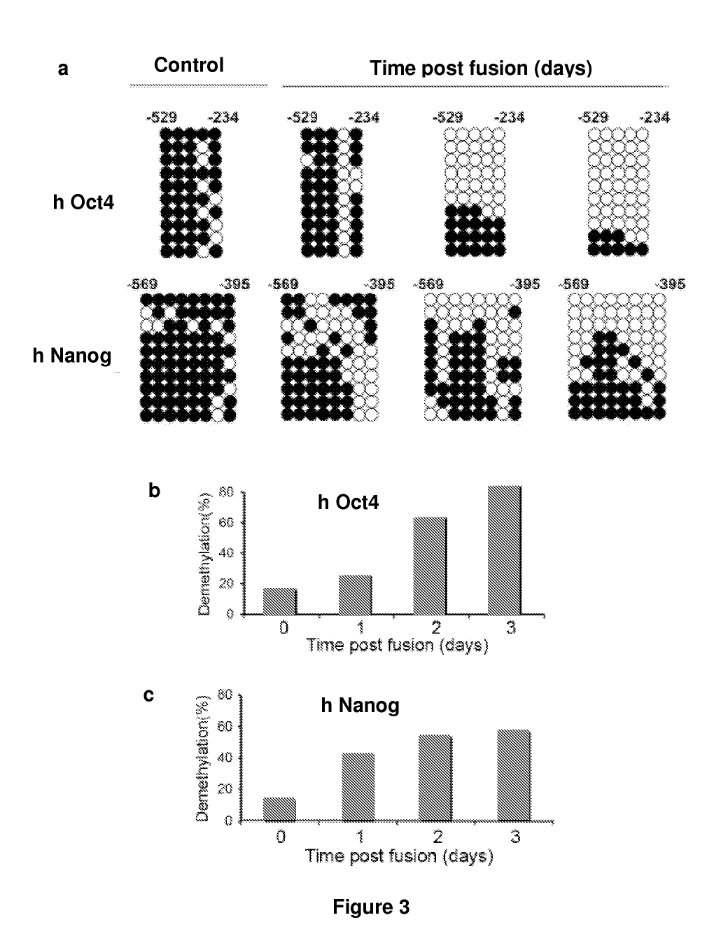

Since DNA demethylation has been shown to be a major limiting step in reprogramming fibroblasts towards iPS cells, the time course and extent of demethylation of the human Oct4 and Nanog promoters in heterokaryons was analyzed relative to control. DNA was isolated from heterokaryons on days 1, 2 and 3 post-fusion and subjected to bisulfite conversion. Human Oct4 and Nanog promoters were amplified by PCR using human- and bisulfite-specific primers (Table 3, FIG. 8), and the products cloned and sequenced. DNA demethylation was evident at the human Oct4 and Nanog promoters and progressively increased through day 3 (FIG. 3a). By contrast, the β-globin HS2 locus remained methylated throughout, indicating that the DNA demethylation was specific. The time-course and progressive accumulation of demethylated CpG sites in the human Oct4 and Nanog promoters (FIG. 3b,c) parallels the progressive increase in transcript accumulation observed over the same three day time period using real time PCR (FIG. 2b). Notably, promoter demethylation and activation of pluripotency genes in human somatic cells takes place in the absence of Ki-67 or BrdU labeling (FIG. 1e,g); thus demethylation is active and independent of cell division and DNA replication.

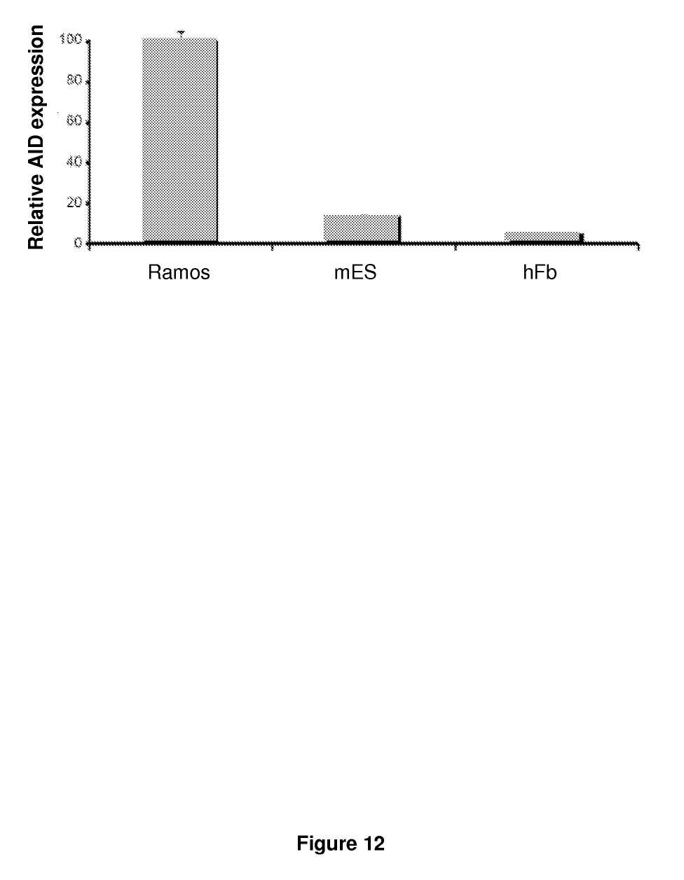

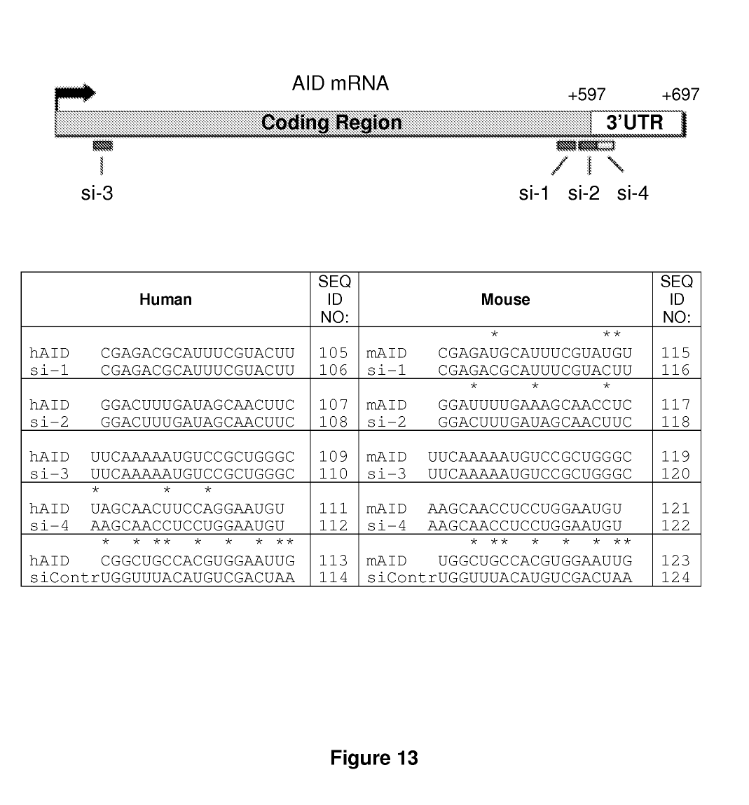

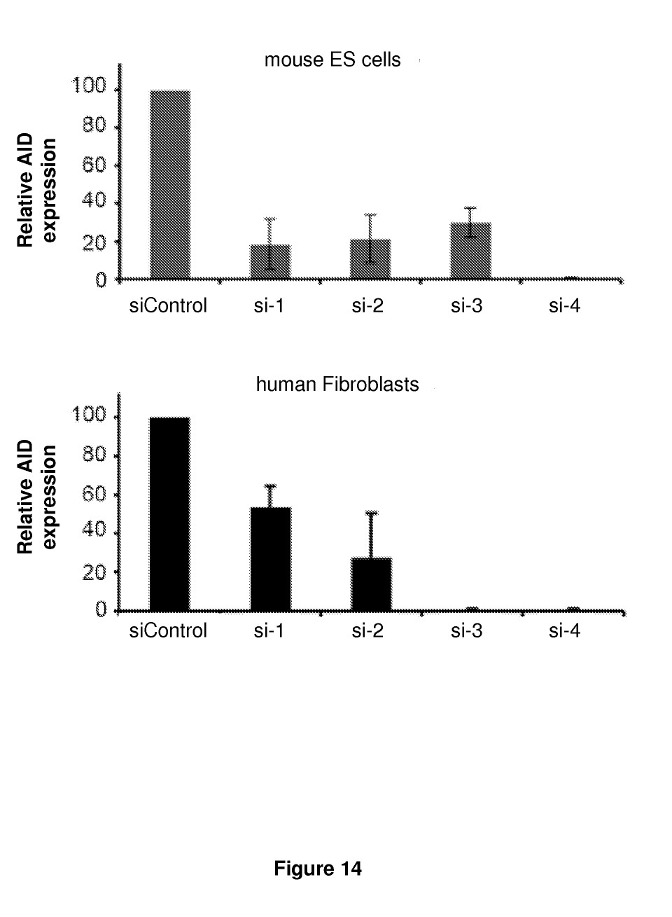

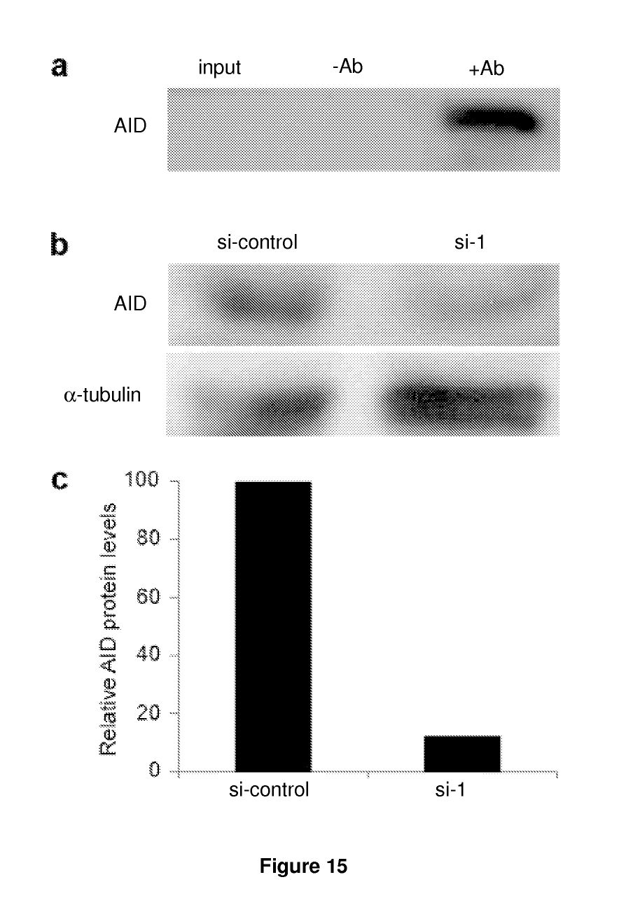

Because is detected in mammalian pluripotent germ cells (Morgan, H. D., et al. (2004) Biol Chem 279, 52353-60) and implicated in active DNA demethylation in zebrafish post fertilization (Rai, K. et al. (2008) Cell 135, 1201-12), mouse ES cells and human fibroblasts were assayed for AID expression using real time PCR. Although AID expression in somatic cells is generally thought to be restricted to B lymphocytes, AID mRNA was detected in human fibroblasts as well as mouse ES cells, albeit at greatly reduced levels (5% and 15%, respectively) compared to Ramos, a B-lymphocyte cell line (FIG. 12). To investigate the role of AID in these cells, mouse and human AID mRNA levels were transiently knocked down by transfection of three distinct, non-overlapping siRNAs to different sequences within the AID coding region, and a fourth siRNA specific to the non-coding 3′UTR of AID, in order to rule out off-target effects and ensure that the results were specific to AID (FIG. 13). A fifth siRNA with 50% identity to the AID coding region was used as a control (siControl). The extent and timing of knockdown was first confirmed in control mouse ES cells in which siRNA-1, 2, 3 and 4 reduced AID transcripts by 81(±13) %, 79(±12) %, 70(±8) %, and 99(±0.1) %, respectively, at day 3 post-transfection as compared to the control siRNA (FIG. 14, top). AID protein was detected in mouse ES cells using immunoprecipitation followed by Western blot as well as in concentrated whole ES cell lysates (FIG. 15). AID knockdown by siRNA 1 was verified in ES cell lysates, and the reduction by 88% of the control protein levels correlated well with the mRNA reduction by 81% (FIG. 15). In human fibroblasts, AID transcripts were reduced by 46(±11)%, 72 (±23) %, 99(±0.1) % and 99(±0.1) % by siRNA 1, 2, 3 and 4, respectively (FIG. 14, bottom). These data show that AID is present and can be efficiently reduced by four distinct siRNAs in both ES cells and fibroblasts.

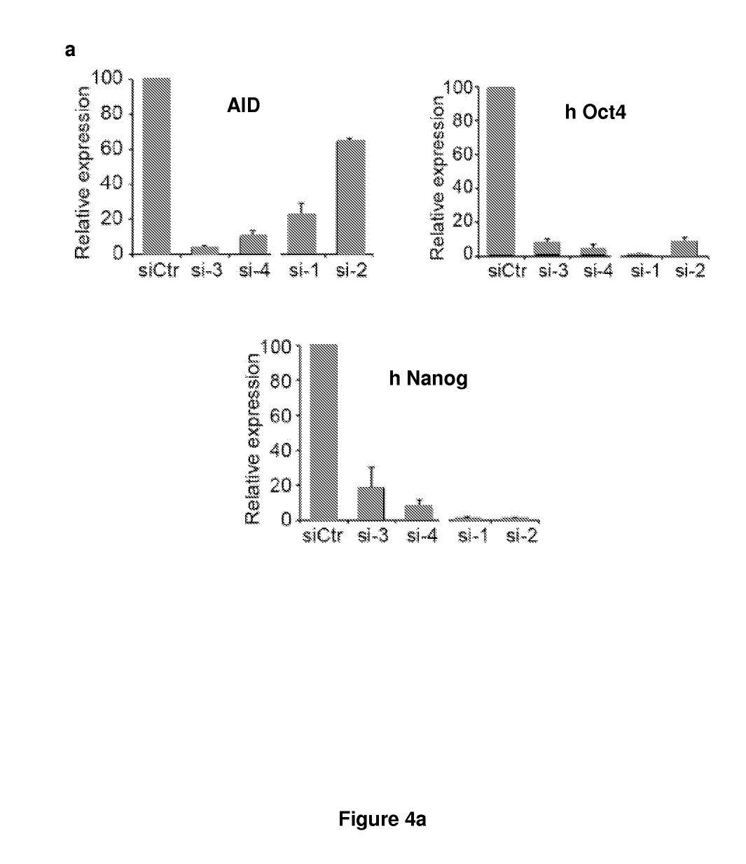

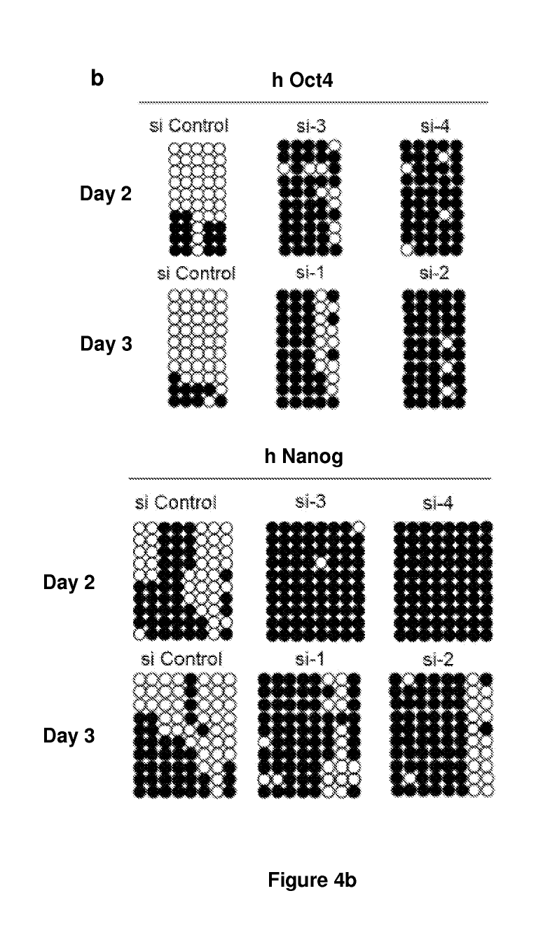

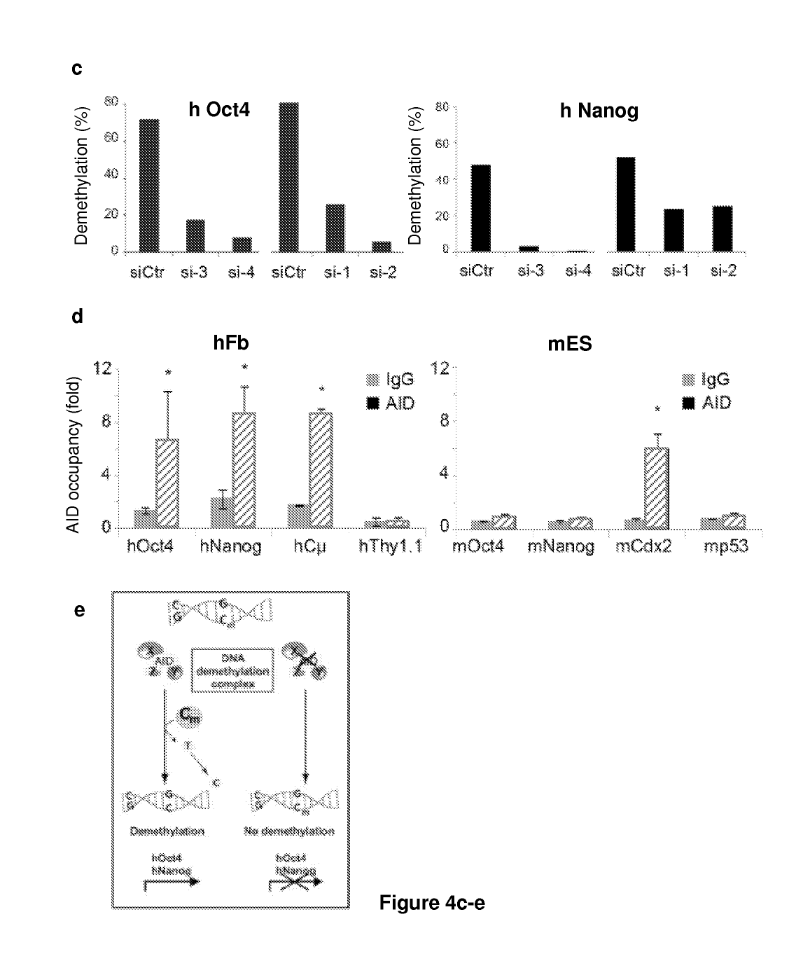

To assess the initiation of reprogramming in heterokaryons subjected to AID knockdown, expression of Oct4 and Nanog relative to GAPDH was assessed by real time PCR. For heterokaryon experiments, siRNAs were transfected into both the mouse ES cells and the human fibroblasts 24 hours prior to fusion (See FIG. 5 for scheme). A persistent knock-down of AID was detected by real time PCR in heterokaryons. Using siRNA 1 and 2, AID was reduced by 77(±6) % and 35(±2) % on day 3 post fusion relative to heterokaryons transfected with the control siRNA (FIG. 4a). The siRNAs 3 and 4 caused a stronger knockdown in heterokaryons with a reduction in AID by 96(±1) %, and 89(±3) % on day 2 post fusion relative to the control siRNA (FIG. 4a). Strikingly, Oct4 expression was reduced to 0.9(±0.6) % and 9(±2) % using siRNA 1 and 2 on day 3 post fusion as compared to the control siRNA (FIG. 4a). Similarly, using siRNA 1 and 2, Nanog expression was greatly reduced to 1.5(±0.4) % and 1.5(±0.1) % on day 3 post fusion relative to the control siRNA (FIG. 4a). In the presence of siRNA 3 and 4, Oct 4 expression was reduced to 8(±2) % and 4(±3) % relative to the control siRNA on day 2 post fusion while Nanog expression was reduced to 19(±12) % and 7(±4) %. All the 4 siRNAs used here had a similar effect in blocking the expression of Oct4 and Nanog by at least 80%. These observations indicate that the effect of AID is extremely dosage sensitive as 35% knockdown led to a comparable inhibition of pluripotency gene induction as a 96% knockdown. Together, these data show that all 4 siRNAs to AID used here had a similarly potent effect in blocking the Oct4 and Nanog activation by at least 80%.

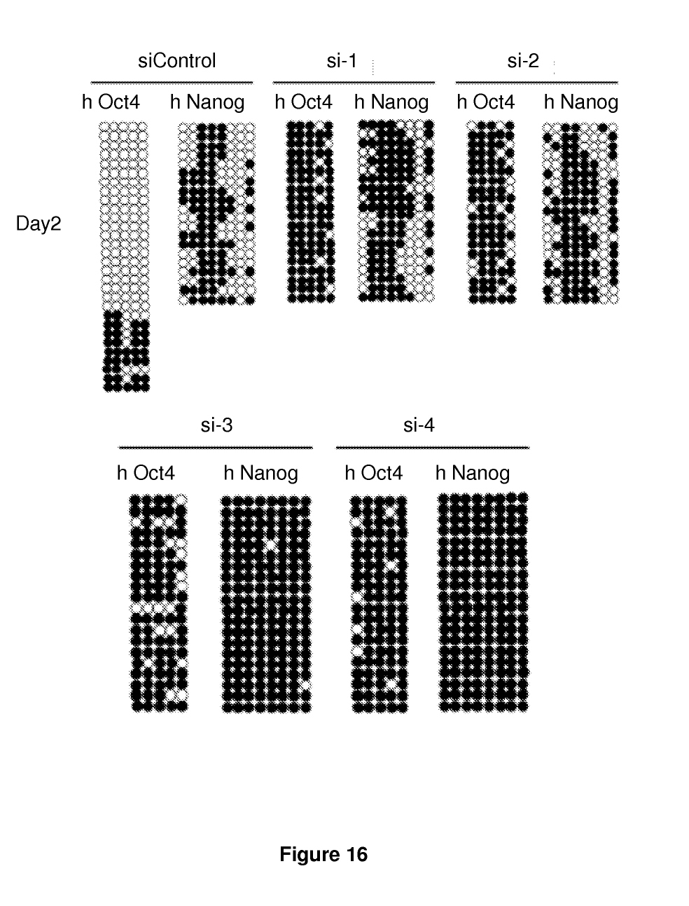

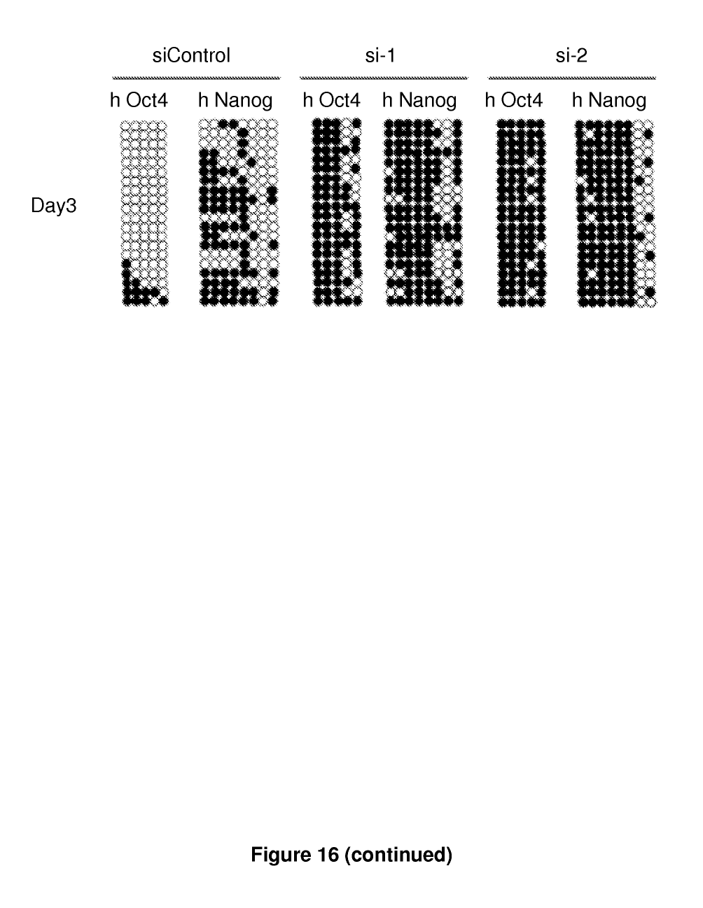

To assess the effect of AID on promoter demethylation, we assayed the CpG methylation status of the human Oct4 and Nanog promoters in heterokaryons. In Day 3 heterokaryons subject to AID knockdown using siRNA 1 and siRNA 2, the extent of CpG demethylation in the human Oct4 promoter was reduced to 26% and 6%, respectively, as compared to the 82% in the control (FIG. 4b,c). For the Nanog promoter, CpG demethylation was reduced to 24% and 25%, respectively, as compared to 53% demethylation for the control (FIG. 4b,c). Using siRNA 3 and 4, the extent of CpG demethylation in the Oct4 promoter was reduced to 18% and 8%, respectively, as compared to 72% in the day 2 control sample, while for the Nanog promoter, the extent of CpG demethylation was reduced to 3% and <1%, respectively, compared to 48% in the control (FIG. 4b,c). A summary of the bisulfite sequencing data for all the siRNA knockdown experiments is shown in FIG. 16. In parallel with the reduction in demethylation of the Oct4 and Nanog promoters upon AID knockdown, the induction of Oct4 and Nanog transcripts was reduced by at least 80% on days 2 and day 3, relative to the control (FIG. 4a). These data show that promoter demethylation is critical to the expression of these two pluripotency genes and that AID is required for mammalian DNA demethylation in somatic cell reprogramming.

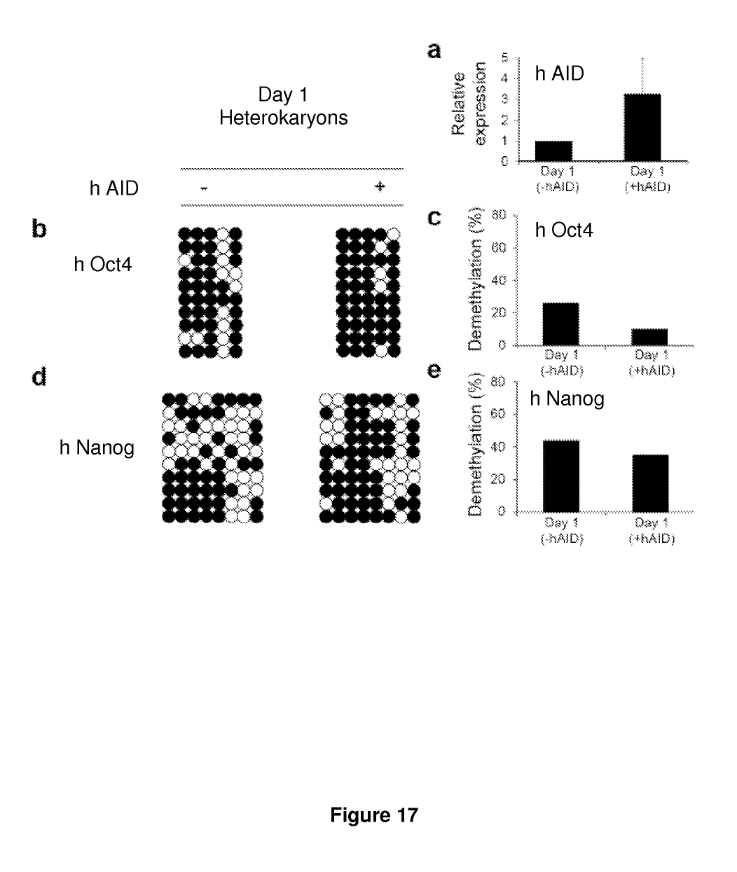

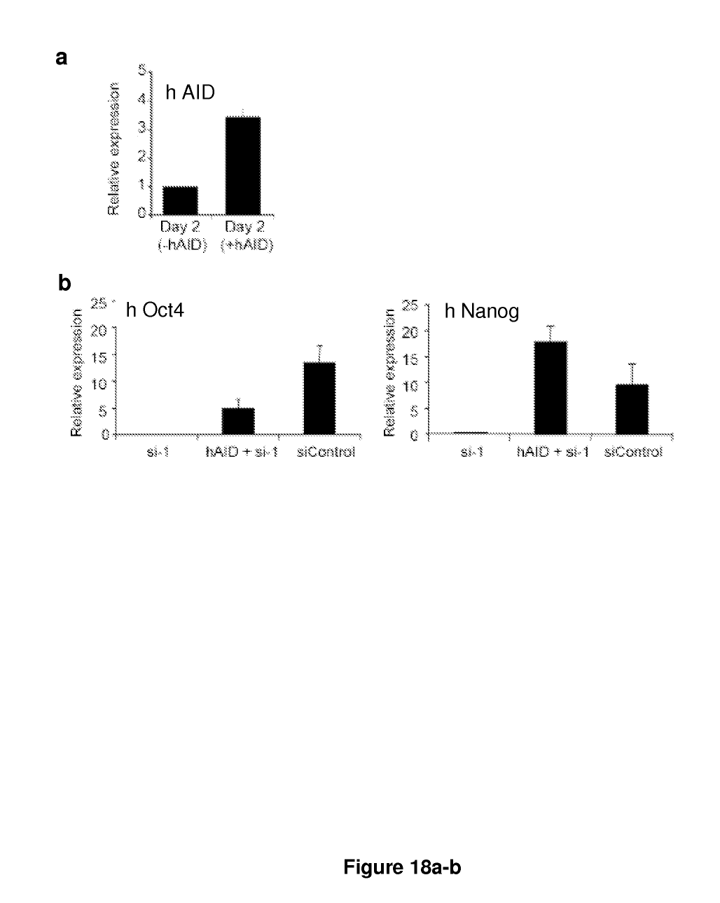

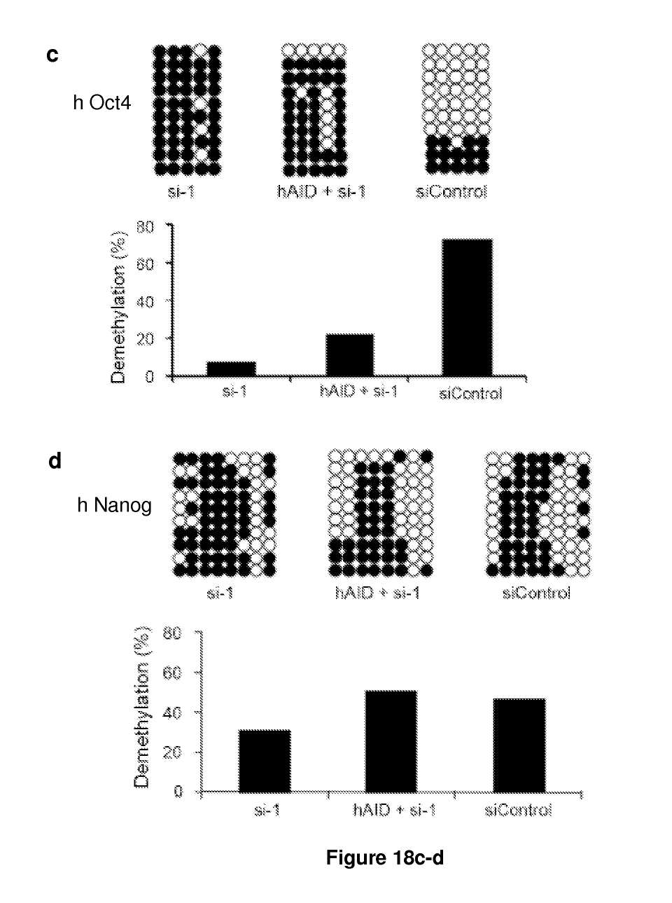

To further investigate the requirement of AID for initiating reprogramming, we tested its ability to rescue the DNA demethylation block caused by the siRNA knockdown in heterokaryons. hAID was transiently overexpressed in mouse ES cells prior to siRNA transfection in order to test whether the siRNA knockdown could be overcome by increasing AID levels (see scheme in FIG. 5). In two separate experiments, when hAID was over-expressed 2-fold or 4-fold relative to the control in heterokaryons in the absence of AID siRNA, there was no acceleration in promoter demethylation or reprogramming at day 1 post fusion (FIG. 17). This could possibly be due to the kinetics of human Oct4 promoter demethylation, which in heterokaryons may require at least 1 day to occur, or by the lack of additional factors that work in concert with hAID to accelerate reprogramming. However, upon overexpression of hAID in heterokaryons undergoing transient knockdown by siRNA-1, i.e., in the presence of siRNA, there was a complete rescue of Nanog promoter demethylation and gene expression and a partial rescue of Oct4 promoter demethylation and gene expression (FIG. 18). These data show that the added hAID is functional and rule out any non-specific effects of the siRNA, further confirming the specific and essential role of AID in DNA demethylation at the onset of reprogramming towards pluripotency.

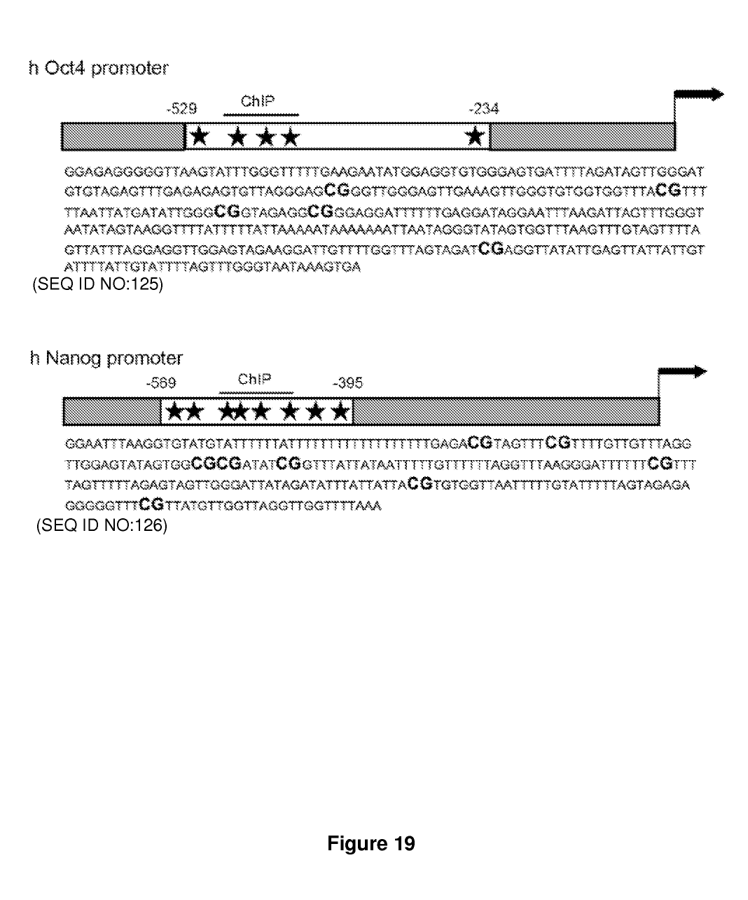

To further validate the role of AID in DNA demethylation of human Oct4 and Nanog promoters, we tested whether AID specifically binds to their promoter regions by performing chromatin immunoprecipitation (ChIP) experiments using an anti-AID antibody. The promoter regions assessed in ChIP experiments were designed to be within the Oct4 and Nanog promoter regions that were analyzed for CpG demethylation by bisulfite sequencing (FIG. 4d; FIG. 19). In the human fibroblasts, the ChIP analyses showed significant binding of AID to both human Oct4 (6-fold) and human Nanog (8-fold) promoters (FIG. 4d). Thus, AID binds to the heavily methylated promoter regions of human Oct4 and Nanog in fibroblasts that undergo demethylation during reprogramming. As controls, AID binding to the promoter of the IgM constant region (Cμ) was significant, as expected (Okazaki, I. M., et al. (2002) Nature 416, 340-5), while no binding was observed for Thy1.1, which is expressed in fibroblasts.

In contrast to fibroblasts, no AID binding was observed at the promoter regions of mouse Oct4 and Nanog despite the higher levels of AID protein in ES cells, presumably because these promoters are expressed and demethylated (FIG. 4d). As controls, AID binding was detected at the promoter of Cdx2, a gene not expressed in undifferentiated ES cells, but was absent from the p53 promoter, as previously reported. Together, these findings provide strong support for a direct involvement of AID in DNA demethylation and the sustained expression of human Nanog and Oct4 leading to the onset of reprogramming towards pluripotency.

### Discussion

DNA demethylation is essential to overcoming gene silencing and inducing temporally and spatially controlled expression of mammalian genes, yet no consensus mammalian DNA demethylase has been identified despite years of effort. Evidence of DNA demethylation via 5 methyl-cytosine DNA glycosylases has been shown in plants (Gong, Z. et al. (2002) Cell 111, 803-14; Choi, Y. et al. (2002) Cell 110, 33-42), but mammalian homologues such as Thymine DNA Glycosylase (TDG) or the Methyl-CpG-binding domain protein 4 (Mbd4) have not exhibited comparable functions (Cortazar, D., et al. (2007) DNA Repair (Amst) 6, 489-504; Millar, C. B. et al. (2002) Science 297, 403-5).

AID belongs to a family of cytosine deaminases (AID, Apobec 1, 2 and 3 subgroups) that have established roles in generating antibody diversity in B cells, RNA editing and antiviral response (Conticello, S. G., et al. (2007) Adv Immunol 94, 37-73). Both AID and Apobec1 are expressed in progenitor germ cells, oocytes and early embryos and have a robust 5-methyl cytosine deaminase activity in vitro (Morgan, H. D., et al. (2004) J Biol Chem 279, 52353-60), resulting in a T-G mismatch that is repaired through the Base Excision DNA Repair (BER) pathway, and could theoretically lead to DNA demethylation without replication. Recently in zebrafish embryos, AID was implicated as a member of a tri-partite protein complex along with Mbd4 and Gadd45a, effecting cytosine deamination and leading to base excision by Mbd4 (Rai, K. et al. (2008) Cell 135, 1201-12). The third component Gadd45a lacks enzymatic activity and its role in repair-mediated DNA demethylation and gene activation in Xenopus oocytes remains a matter of debate (Barreto, G. et al. (20070 Nature 445, 671-5; Jin, S. G., et al. (2008) PLoS Genet. 4, e1000013).

The data provide herein provides evidence implicating AID in active DNA demethylation in mammalian cells and demonstrating that AID-dependent DNA demethylation is an early epigenetic change necessary for the induction of pluripotency in human fibroblasts. Knockdown of AID in heterokaryons prevented DNA demethylation of the human Oct4 and Nanog promoters in fibroblast nuclei. Consistent with this, the expression of these pluripotency factors and the initiation of nuclear reprogramming towards pluripotency was inhibited in human somatic fibroblasts when AID-dependent DNA demethylation was reduced, providing strong evidence that AID is a regulator crucial to the onset of reprogramming. The inhibitory effects of AID reduction were rescued by hAID over-expression, with a complete rescue observed for Nanog and a partial rescue observed for Oct4. Moreover, AID binding was observed at silent methylated Oct4 and Nanog promoters in human fibroblasts but not in active unmethylated Oct4 and Nanog promoters in mouse ES cells, demonstrating its specific role in DNA demethylation.

The high efficiency of reprogramming in heterokaryons achieved here allowed the discovery of a regulator critical to the induction of five pluripotency genes including Oct4 and Nanog, the first known markers of stable reprogramming leading to the generation of iPS cells. The heterokaryon platform can now be exploited (a) to elucidate the other components of the mammalian DNA demethylation complex (glycosylase and other DNA repair enzymes) that are likely to work together with AID to mediate active DNA-demethylation (FIG. 4e) and (b) to perform an unbiased search for additional regulators of nuclear reprogramming by screening for human genes that are immediately expressed after cell fusion. Future studies will reveal whether expression of AID alone or in conjunction with these other molecules will accelerate the generation of iPS cells.

### Example 2

Mass spectrometry was used to identify the potential interactors of AID and understand the functional molecular players that orchestrate mammalian DNA demethylation. The following AID constructs were used: 1) human AID containing two tandem Flag tags at the N-terminus of the protein, cloned into the pHAGE-STEMCCA lentiviral vector, and 2) human AID containing two tandem Flag tags at the C-terminus of the protein, cloned into the pHAGE-STEMCCA lentiviral vector. Virus containing these constructs was subsequently used to infect mouse embryonic stem cells (CGR8), and stable cell lines overexpressing Flag-human AID were selected. As a control, the lentiviral vector containing only the 2× Flag tag was used.

The stable ES cell lines expressing AID and Control 2× Flag were fractionated into cytoplasmic and nuclear extracts for immunoprecipitating the AID protein using an antibody against the Flag tag. The resulting complex was subjected to mass spectrometric analyses. In the analyses, AID was found to be the most abundant protein, and a number of unique proteins associated with AID were identified (Table 5).

TABLE 5
After immunoprecipitation, AID and the interacting proteins were
identified by mass spectrometry (MS). All proteins were subjected
to trypsin digestion to break them down into smaller peptides, and
run through a mass spectrometer. Column C indicates the number
of unique peptides of a particular protein detected by MS analysis,
to be associated with the AID-Flag protein. Column D represents
the number of peptides associated with the Flag only protein. The
higher the number of unique peptides (>3) that are associated with
AID, but not with the Flag protein, the stronger the indication of the
specificity of the interaction. Columns E and F represent the
associated spectra i.e. the frequency of these peptides detected in
association with the AID protein as a measure of the abundance of
the associated protein.

| | Nucleus peptide | | Nucleus spectra | |
|---|---|---|---|---|
| | EXPERI- | | EXPERI- | |
| GENE NAME | MENTAL | CONTROL | MENTAL | CONTROL |
| Tet1 | 14 | 1 | 41 | |
| Mdn1 | 54 | 5 | 119 | 5 |
| Aicda | 5 | 0 | 35 | 0 |
| Aicda | 8 | 1 | 44 | 2 |
| Ncapd2 | 10 | 1 | 22 | 1 |
| Dnaja2 | 12 | 1 | 43 | 2 |
| Dnaja3 | 7 | 1 | 28 | 2 |
| Nol9 | 6 | 1 | 14 | 1 |
| Ranbp2 | 57 | 8 | 160 | 12 |
| Hells | 6 | 1 | 13 | 1 |
| Bst2 | 4 | 1 | 12 | 1 |
| Psmd2 | 5 | 1 | 12 | 1 |
| Canx | 7 | 2 | 23 | 2 |
| Supt5h | 6 | 1 | 11 | 1 |
| Pfkl | 6 | 1 | 10 | 1 |
| Sall4 | 6 | 1 | 10 | 1 |
| Emd | 4 | 1 | 10 | 1 |
| Dnaja1 | 13 | 3 | 49 | 5 |
| Zfp281 | 14 | 3 | 49 | 5 |
| Nasp | 5 | 1 | 9 | 1 |
| Dnajb6 | 5 | 1 | 17 | 2 |
| Gm1040 | 5 | 1 | 8 | 1 |
| Smarcad1 | 5 | 1 | 8 | 1 |
| Zc3h18 | 2 | 1 | 8 | 1 |
| Las1l | 9 | 2 | 21 | 3 |
| Psmc2 | 3 | 1 | 7 | 1 |
| Ddx20 | 3 | 1 | 7 | 1 |
| Mcm4 | 6 | 2 | 13 | 2 |
| Rif1 | 37 | 8 | 109 | 17 |
| Plekha7 | 4 | 1 | 6 | 1 |
| Ywhae | 3 | 1 | 6 | 1 |
| Akr1b3 | 4 | 2 | 11 | 2 |
| Mllt4 | 7 | 1 | 11 | 2 |
| Lmnb2 | 10 | 3 | 27 | 5 |
| Nars | 7 | 3 | 16 | 3 |
| Aars | 10 | 3 | 21 | 4 |
| Tubb6 | 3 | 1 | 5 | 1 |
| Pum2 | 2 | 1 | 5 | 1 |
| Col18a1 | 4 | 1 | 5 | 1 |
| Hdlbp | 3 | 1 | 5 | 1 |
| Vdac1 | 4 | 1 | 5 | 1 |
| Gcn1l1 | 14 | 3 | 24 | 5 |
| Plec1 | 48 | 15 | 94 | 20 |
| Smarca1 | 7 | 2 | 14 | 3 |
| Ssrp1 | 9 | 2 | 18 | 4 |
| Cttn | 4 | 1 | 9 | 2 |
| LOC100045999; | 3 | 1 | 9 | 2 |
| Ran | | | | |
| Chd4 | 15 | 4 | 26 | 6 |
| Pou5f1 | 4 | 2 | 13 | 3 |
| Lmna | 6 | 1 | 13 | 3 |
| Cct2 | 8 | 2 | 17 | 4 |
| Mcm2 | 9 | 4 | 21 | 5 |
| Hspd1 | 16 | 5 | 44 | 11 |
| Tex10 | 6 | 1 | 12 | 3 |
| Cbx5 | 4 | 2 | 12 | 3 |
| Smarca4 | 5 | 2 | 8 | 2 |
| Sfrs17b | 3 | 2 | 8 | 2 |
| Tmem48 | 3 | 1 | 4 | 1 |
| Ttf1 | 3 | 1 | 4 | 1 |
| Abce1 | 7 | 3 | 11 | 3 |
| Uba1 | 10 | 3 | 18 | 5 |
| Smc4 | 5 | 1 | 7 | 2 |
| Eno2 | 3 | 1 | 7 | 2 |
| Ranbp1 | 2 | 1 | 7 | 2 |
| Msh2 | 26 | 9 | 78 | 23 |
| Zc3h18 | 5 | 3 | 20 | 6 |
| Krt18 | 21 | 13 | 96 | 29 |
| Kpnb1 | 15 | 5 | 43 | 13 |
| Seh1l | 5 | 2 | 13 | 4 |
| Mcm3 | 9 | 5 | 22 | 7 |
| Spna2 | 14 | 5 | 28 | 9 |
| Ruvbl2 | 12 | 4 | 31 | 10 |
| Cct8 | 12 | 6 | 31 | 10 |
| Smarca5 | 16 | 8 | 45 | 15 |
| Cbx1 | 2 | 1 | 12 | 4 |
| Atxn2l | 4 | 2 | 9 | 3 |
| Dars | 4 | 1 | 6 | 2 |
| Prdx6 | 3 | 1 | 3 | 1 |
| Pkm2 | 3 | 1 | 3 | 1 |
| Ywhaz | 6 | 1 | 17 | 6 |
| Cnot1 | 6 | 3 | 11 | 4 |
| Kif23 | 4 | 3 | 11 | 4 |
| Eef2 | 13 | 6 | 24 | 9 |
| Smc2 | 7 | 4 | 16 | 6 |
| Utf1 | 7 | 4 | 16 | 6 |
| Tmpo | 4 | 2 | 16 | 6 |
| Rcc1 | 7 | 3 | 16 | 6 |
| Upf1 | 5 | 2 | 8 | 3 |
| Nup214 | 13 | 7 | 29 | 11 |
| Tip2 | 16 | 6 | 36 | 14 |
| Alpl | 12 | 5 | 25 | 10 |
| Dnmt3l | 4 | 2 | 10 | 4 |
| Smarcc1 | 6 | 4 | 10 | 4 |
| Prpf4 | 4 | 2 | 5 | 2 |
| Peg10 | 3 | 2 | 5 | 2 |
| Tcp1 | 3 | 1 | 5 | 2 |
| Uba2 | 5 | 2 | 5 | 2 |
| Nsd1 | 4 | 1 | 5 | 2 |
| Mcm6 | 13 | 8 | 29 | 12 |
| Spnb2 | 17 | 7 | 26 | 11 |
| Pfn1 | 5 | 4 | 21 | 9 |
| Rps6ka1 | 3 | 2 | 7 | 3 |
| Racgap1 | 6 | 3 | 7 | 3 |
| Gart | 5 | 2 | 7 | 3 |
| Eprs | 5 | 3 | 7 | 3 |
| Nup188 | 4 | 2 | 7 | 3 |
| Hspa5 | 13 | 10 | 30 | 13 |
| Atxn10 | 10 | 4 | 23 | 10 |
| Tufm | 9 | 6 | 25 | 11 |
| Hmga1 | 5 | 1 | 18 | 8 |
| Copa | 5 | 1 | 9 | 4 |
| Ctbp2 | 4 | 2 | 9 | 4 |
| Cct6a | 9 | 6 | 20 | 9 |
| Nup160 | 12 | 6 | 24 | 11 |
| Ruvbl1 | 13 | 9 | 37 | 17 |
| Impdh2 | 12 | 8 | 37 | 17 |
| Lars | 9 | 4 | 13 | 6 |
| Pcbp1 | 5 | 2 | 13 | 6 |
| Rbm25 | 6 | 3 | 15 | 7 |
| Smpd4 | 9 | 4 | 17 | 8 |
| Wapal | 18 | 0 | 41 | 0 |
| Aicda | 5 | 0 | 35 | 0 |
| Lmo7 | 15 | 0 | 31 | 0 |
| Rangap1 | 10 | 0 | 29 | 0 |
| Aff4 | 13 | 0 | 25 | 0 |
| Mtap1b | 11 | 0 | 24 | 0 |
| Smc3 | 12 | 0 | 23 | 0 |
| Dync1h1 | 16 | 0 | 21 | 0 |
| Kif23 | 7 | 0 | 18 | 0 |
| Ahdc1 | 8 | 0 | 15 | 0 |
| Dnmt3a | 6 | 0 | 13 | 0 |
| Etl4 | 7 | 0 | 13 | 0 |
| Ogt | 7 | 0 | 12 | 0 |
| Akap8 | 4 | 0 | 12 | 0 |
| Kars | 6 | 0 | 9 | 0 |
| Zfp655 | 4 | 0 | 9 | 0 |
| Dnmt3b | 4 | 0 | 9 | 0 |
| Ssr1 | 3 | 0 | 9 | 0 |
| Ncapg | 4 | 0 | 9 | 0 |
| Rfc2 | 3 | 0 | 8 | 0 |
| Psmc5 | 5 | 0 | 8 | 0 |
| Cpsf1 | 4 | 0 | 8 | 0 |
| Cul4b | 4 | 0 | 7 | 0 |
| Ywhab | 3 | 0 | 7 | 0 |
| Prpf4b | 3 | 0 | 7 | 0 |
| Pcnt | 5 | 0 | 6 | 0 |
| Rad21 | 3 | 0 | 6 | 0 |
| Aff1 | 3 | 0 | 6 | 0 |
| Mdn1 | 3 | 0 | 6 | 0 |
| Xpo1 | 4 | 0 | 6 | 0 |
| Calu | 3 | 0 | 6 | 0 |
| Cct7 | 3 | 0 | 5 | 0 |
| Rfc5 | 3 | 0 | 5 | 0 |
| Pum1 | 2 | 0 | 5 | 0 |
| Rpn1 | 3 | 0 | 5 | 0 |
| Ints3 | 4 | 0 | 5 | 0 |
| Cse1l | 3 | 0 | 4 | 0 |
| Kif11 | 3 | 0 | 4 | 0 |
| Ddb1 | 3 | 0 | 3 | 0 |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of decreasing the amount of genomic DNA methylation in a mammalian cell, comprising:
contacting said mammalian cell with an effective amount of one or more agents that promote cytidine deaminase (CD) activity conditions such that genomic DNA methylation is decreased.

2. The method according to claim 1, wherein the one or more agents that promote CD activity is an Activation-induced Cytidine Deaminase (AID) polypeptide or a nucleic acid encoding an AID polypeptide.

3. The method according to claim 1, wherein the one or more agents that promote CD activity is an Apolipoprotein B RNA Editing Catalytic Component (APOBEC) polypeptide or a nucleic acid encoding an APOBEC peptide.

4. The method according to claim 1, further comprising the step of contacting said mammalian cell with a tet protein.

5. The method according to claim 1, wherein said contacting step is effected in vitro.

6. The method according to claim 1, wherein said mammalian cell that is contacted is a demethylation-permissive somatic cell.

7. The method according to claim 6, wherein the mammalian cell that is contacted becomes an induced pluripotent stem (iPS) cell following said contacting step.

8. The method according to claim 7, wherein the method further comprises the step of contacting said demethylation-permissive somatic cell with one or more factors that promote an iPS cell fate.

9. The method according to claim 6, wherein the mammalian cell that is contacted becomes a somatic cell of a different cell lineage than that of the demethylation-permissive somatic cell.

10. The method according to claim 9, wherein the method further comprises the step of contacting said demethylation-permissive somatic cell with one or more factors that promote a desired somatic cell fate.

11. The method according to claim 1, wherein the mammalian cell that is contacted is a pluripotent stem cell.

12. The method according to claim 11, wherein the pluripotent stem cell is selected from the group consisting of an embryonic stem (ES) cell, an embryonic germ stem (EG) cell, and an induced pluripotent stem (iPS) cell.

13. The method according to claim 11, wherein the cell that is produced is a somatic cell.

14. The method according to claim 13, wherein the method further comprises the step of contacting said pluripotent cell with one or more factors that promote a desired somatic cell fate.

15. The method according to claim 1, wherein said contacting step is effected in a subject in need of genomic DNA demethylation therapy.

16. The method according to claim 15, wherein said cell is a cancer cell and said subject is a subject suffering from cancer.

17. A method of screening candidate agents for activity in modulating genomic DNA demethylation activity in a cell, the method comprising: contacting a population of cells with an effective amount of an agent that promotes cytidine deaminase (CD) activity,
comparing the candidate-agent contacted cells with a population of cells that have been contacted with an agent that promotes cytidine deaminase activity but that have not been contacted with the candidate agent,
wherein differences in the characteristics between the first subpopulation and the second subpopulation indicate that the candidate agent modulates genomic DNA demethylation activity.

18. The method according to claim 17, wherein the agent that promotes CD activity is an AID peptide or a nucleic acid that encodes an AID peptide.

19. The method according to claim 17, wherein said first population of cells are tumor cells.

20. The method according to claim 19, wherein a candidate agent that modulates genomic demethylation in the tumor cells is an agent that modulates tumor growth in a cancer.

21. The method according to claim 17, wherein said first population of cells are somatic cells or heterokaryons produced from ES cells and somatic cells.

22. The method according to claim 21, wherein a candidate agent that modulates genomic demethylation in the somatic cells is an agent that modulates the induction of pluripotency of the somatic cell.

23. A method for identifying a protein with activity in modulating the DNA demethylation activity of a cytidine deaminase, the method comprising:
contacting a population of cells with a nucleic acid comprising sequence encoding the cytidine deaminase,
immunoprecipitating the cytidine deaminase from a crude protein extract of said cells, and
subjecting said immunoprecipitate to mass spectroscopy,
wherein the one or more proteins identified by mass spectroscopy is critical to the demethylation activity of said cytidine deaminase.
